(19) United States
(12) Patent Application Publication
(10) Pub. No.: US 2023/0390466 A1
(43) Pub. Date: Dec. 7, 2023

(54) SYSTEMS AND METHODS FOR OPTIMIZING TREATMENT USING PHYSIOLOGICAL PROFILES

(71) Applicant: Dexcom, Inc., San Diego, CA (US)

(72) Inventors: Matthew L. JOHNSON, San Diego, CA (US); Qi AN, San Diego, CA (US); Rush BARTLETT, San Diego, CA (US); John PADERI, San Diego, CA (US)

(21) Appl. No.: 18/327,014

(22) Filed: May 31, 2023

Related U.S. Application Data
(60) Provisional application No. 63/365,702, filed on Jun. 1, 2022.
(60) Provisional application No. 63/376,673, filed on Sep. 22, 2022.
(60) Provisional application No. 63/387,078, filed on Dec. 12, 2022.
(60) Provisional application No. 63/377,332, filed on Sep. 27, 2022.
(60) Provisional application No. 63/403,568, filed on Sep. 2, 2022.
(60) Provisional application No. 63/403,582, filed on Sep. 2, 2022.

(51) Int. Cl. A61M 1/16

(52) U.S. Cl. CPC A61M 1/1613; A61M 1/1603; A61M 2230/201; A61B 5/14546

(57) ABSTRACT

Certain aspects of the present disclosure provide a monitoring system comprising a continuous analyte sensor configured to generate analyte measurements associated with analyte levels of a patient, and a sensor electronics module coupled to the continuous analyte sensor and configured to receive and process the analyte measurements.

### CROSS-REFERENCE TO RELATED APPLICATIONS

This application claims priority to and benefit of U.S. Provisional Application No. 63/365,702, filed Jun. 1, 2022, and U.S. Provisional Application No. 63/376,673, filed Sep. 22, 2022, and U.S. Provisional Application No. 63/387,078, filed Dec. 12, 2022, and U.S. Provisional Application No. 63/377,332, filed Sep. 27, 2022, and U.S. Provisional Application No. 63/403,568, filed Sep. 2, 2022, and U.S. Provisional Application No. 63/403,582, filed Sep. 2, 2022, which are hereby assigned to the assignee hereof and hereby expressly incorporated by reference in their entirety as if fully set forth below and for all applicable purposes.

### BACKGROUND

The kidney is responsible for many critical functions within the human body including, filtering waste and excess fluids, which are excreted in the urine, and removing acid that is produced by the cells of the body to maintain a healthy balance of water, salts, and minerals (e.g., such as sodium, calcium, phosphorus, and potassium) in the blood. In other words, the kidney plays a major role in homeostasis by renal mechanisms that transport and regulate water, salt, and mineral secretion, reabsorption, and excretion. Further, kidneys secrete renin (e.g., angiotensinogenase), which forms part of the renin-angiotensin-aldosterone system (RAAS) that mediates extracellular fluid and arterial vasoconstriction (e.g., blood pressure). More specifically, high blood pressure (e.g., hypertension) can be regulated through RAAS inhibitors such as angiotensin-converting enzyme (ACE) inhibitors and angiotensin receptor blockers (ARBs). Should the kidney become diseased or injured, the impairment or loss of these functions can cause significant damage to the human body.

Kidney disease occurs when the kidney becomes diseased or injured. Kidney disease is generally classified as either acute or chronic based upon the duration of the disease. Acute kidney injury (AKI) (also referred to as “acute renal failure”) is usually caused by an event that leads to kidney malfunction, such as dehydration, blood loss from major surgery or injury, and/or the use of medicines. On the other hand, chronic kidney disease (CKD) is usually caused by a long-term disease, such as high blood pressure or diabetes, which slowly damages the kidneys and reduces their function over time.

Conventional kidney disease diagnostic methods and systems include albumin-to-creatinine ratio (ACR) tests, glomerular filtration rate (GFR) tests, and blood tests for monitoring potassium levels of a patient. CKD is divided into five stages based on the severity of kidney dysfunction, as measured by the various methods and systems. Kidney disease in stages 1-3a is mild to moderate kidney dysfunction. Kidney disease in stages 3b-5 is moderate to severe kidney dysfunction. End stage renal disease (ESRD) is total kidney dysfunction or kidney failure.

The GFR method for diagnosis and staging of kidney disease represents the flow rate of filtered fluid through the kidney. Creatinine clearance rate is the volume of blood plasma that is cleared of creatinine per unit of time and is used to approximate the GFR. GFR can be measured (e.g., measured GFR (mGFR)) with gold standard methods or estimated (e.g., eGFR) with formulas. EGFR provides a more convenient and rapid analysis for evaluating kidney function.

In some cases, when CKD is left untreated, elevated potassium levels of a patient with CKD may lead to hyperkalemia, while lower potassium levels of a patient with CKD may lead to hypokalemia. In particular, hyperkalemia is the medical term that describes a potassium level in the blood that is higher than normal (e.g., higher than normal blood potassium levels between 3.6 and 5.2 millimoles per liter (mmol/L)). Hyperkalemia increases the risk of cardiac arrhythmia episodes and sudden death. On the other hand, hypokalemia is the medical term that describes a potassium level in the blood that is lower than normal. In particular, CKD patients may develop hypokalemia due to gastrointestinal potassium loss from diarrhea or vomiting or renal potassium loss from non-potassium-sparing diuretics (e.g., diuretics used to increase the amount of fluid passed from the body in urine, without regard for the amount of potassium being lost from the body in the urine). Severe hypokalemia and hyperkalemia may lead to severe symptoms of respiratory failure, sudden cardiac death, or other mortality-driven event.

The kidney also plays an important role in regulation of blood glucose. The kidney raises blood glucose levels by generating glucose, via gluconeogenesis, and releasing glucose into the blood. The kidney also lowers blood glucose levels by reabsorbing glucose at the proximal tubule of the kidney. Additionally, the kidney uses glucose as an energy source.

Glucose is a simple sugar (e.g., a monosaccharide). Glucose can be both ingested, as well as, produced in the body from protein and carbohydrates. Serum glucose is maintained at healthy levels (e.g., glucose homeostasis) through several mechanisms. High blood glucose (i.e., hyperglycemia) is reduced by insulin and cleared by the kidney. Low blood glucose (i.e., hypoglycemia) is raised by gluconeogenesis in the kidney and liver.

Increasing blood glucose levels stimulate insulin release. Insulin causes the cells to take in glucose, thereby reducing serum (e.g., extracellular) glucose levels to maintain glucose homeostasis. Insulin also stimulates potassium uptake by cells, thereby reducing serum potassium levels. In some cases, where glucose levels of a patient are increased and rate(s) of change of glucose levels in the patient's body are high, excess insulin may be produced thereby causing excess movement of potassium intracellularly. On the other hand, where glucose levels of a patient are decreased and rate(s) of change of glucose levels in the patient's body are low, there may be less insulin secretion. In certain cases, low insulin may lead to limited access of glucose and potassium by the cells; thus, extracellular glucose and potassium levels may increase. In certain other cases, a patient with diabetes may have high insulin levels and high glucose levels, although the high levels of insulin may be ineffective in metabolizing glucose because of the patient's potential insulin resistance. On the other hand, high insulin levels in a diabetes patient may drive potassium into cells, decreasing potassium levels (e.g., regulating potassium levels) for a patient administering insulin.

Gluconeogenesis is the formation of glucose from precursor molecules (e.g., lactate, glycerol, and/or amino acids). Glucose is formed in the kidney and liver, and then released into circulation. Gluconeogenesis is a mechanism to maintain glucose homeostasis by preventing low blood glucose (i.e., hypoglycemia). As kidney function declines, gluconeogenesis in the kidney declines, and thus limits the kidney's ability to react to falling blood glucose.

Diabetes mellitus is a disorder in which the pancreas cannot create insulin (Type I or insulin dependent) and/or in which insulin is not as effective or not produced in sufficient amounts to lower blood sugar to a normal state (Type 2 or non-insulin dependent). In the diabetic state, the patient suffers from high blood sugar, which causes an array of physiological derangements (e.g., kidney failure, skin ulcers, or bleeding into the vitreous of the eye) associated with the deterioration of small blood vessels. A hypoglycemic reaction (i.e., low blood sugar) can be induced by an inadvertent overdose of insulin, or after a normal dose of insulin or glucose lowering agent, or insufficient food intake. Treatment for diabetes requires maintenance of glucose homeostasis. Glucose levels may be controlled through a variety of medications, including exogenous insulin.

In some cases, a patient may suffer from insulin resistance. Insulin resistance occurs when cells in the patient's muscles, fat, and liver do not respond well to insulin. Accordingly, glucose metabolism, as well as potassium movement intracellularly may be impaired. As a result, the patient's pancreas makes more insulin to help glucose and insulin enter the patient's cells. Further, the effect of insulin resistance on glucose metabolism may be different for different patients.

Many medications may also affect kidney function or otherwise be affected by kidney function. Some medications or medical treatments may replace or supplement kidney function, such as dialysis and diuretics. Other medications or medical treatments may reduce kidney function, such as nonsteroidal anti-inflammatory drugs (NSAIDS) such as ibuprofen (e.g., Advil, Motrin) and naproxen (e.g., Aleve), vancomycin, iodinated radiocontrast (e.g., refers to any contrast dyes used in diagnostic testing), angiotensin-converting enzyme (ACE) such as lisinopril, enalapril, and ramipril, aminoglycoside antibiotics such as neomycin, gentamicin, tobramycin, and amikacin, antiviral human immunodeficiency virus (HIV) medications, zoledronic acid (e.g., Zometa, Reclast), foscarnet, and the like. Further, some medical treatments may be affected by changes in kidney function, such as insulin and statins. Although medications and/or medical treatments may be known to affect or be affected by kidney function, it may be nonetheless desirable to continue use of such medications.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the drawings. It is to be noted, however, that the appended drawings illustrate only certain typical aspects of this disclosure and are therefore not to be considered limiting of its scope, for the description may admit to other equally effective aspects.

FIG. 1 illustrates aspects of an example decision support system that may be used in connection with implementing embodiments of the present disclosure.

FIG. 2 is a diagram conceptually illustrating an example continuous analyte monitoring system including example continuous analyte sensor(s) with sensor electronics, in accordance with certain aspects of the present disclosure.

FIG. 3 illustrates example inputs and example metrics that are calculated based on the inputs for use by the decision support system of FIG. 1, according to some embodiments disclosed herein.

FIG. 4 is an example method for providing decision support, according to certain embodiments disclosed herein.

FIG. 5 is a flow diagram depicting a method for training machine learning models to generate predictions on a risk of adverse events and generate treatment decisions and/or recommendations to address the identified risk, according to certain embodiments disclosed herein.

FIG. 6 is a block diagram depicting a computing device configured to perform the operations of FIGS. 4 and 5, according to certain embodiments disclosed herein.

FIGS. 7A-7B depict exemplary enzyme domain configurations for a continuous multi-analyte sensor, according to certain embodiments disclosed herein.

FIGS. 7C-7D depict exemplary enzyme domain configurations for a continuous multi-analyte sensor, according to certain embodiments disclosed herein.

FIG. 7E depicts an exemplary enzyme domain configuration for a continuous multi-analyte sensor, according to certain embodiments disclosed herein.

FIGS. 8A-8B depict alternative views of an exemplary dual electrode enzyme domain configuration for a continuous multi-analyte sensor, according to certain embodiments disclosed herein.

FIGS. 8C-8D depict alternative views of an exemplary dual electrode enzyme domain configuration for a continuous multi-analyte sensor, according to certain embodiments disclosed herein.

FIG. 8E depicts an exemplary dual electrode configuration for a continuous multi-analyte sensor, according to certain embodiments disclosed herein.

FIG. 9A depicts an exemplary enzyme domain configuration for a continuous multi-analyte sensor, according to certain embodiments disclosed herein.

FIGS. 9B-9C depict alternative exemplary enzyme domain configurations for a continuous multi-analyte sensor, according to certain embodiments disclosed herein.

FIG. 10 depicts an exemplary enzyme domain configuration for a continuous multi-analyte sensor, according to certain embodiments disclosed herein.

FIGS. 11A-11D depict alternative views of exemplary dual electrode enzyme domain configurations G1-G4 for a continuous multi-analyte sensor, according to certain embodiments disclosed herein.

FIGS. 12A-12B schematically illustrate an example configuration and component of a device for measuring an electrophysiological signal and/or concentration of a target ion in a biological fluid in vivo, according to certain embodiments disclosed herein.

FIG. 13 schematically illustrates additional example configurations and component of a device for measuring an electrophysiological signal and/or a concentration of a target ion in a biological fluid in vivo, according to certain embodiments disclosed herein.

FIGS. 14A-14C schematically illustrate example configurations and components of a device for measuring an electrophysiological signal and/or concentration of a target analyte in a biological fluid in vivo, according to certain embodiments disclosed herein.

FIG. 15 is a diagram depicting an example continuous analyte monitoring system configured to measure target ions and/or other analytes as discussed herein, according to certain embodiments disclosed herein.

To facilitate understanding, identical reference numerals have been used, where possible, to designate identical elements that are common to the figures. It is contemplated that elements disclosed in one aspect may be beneficially utilized on other aspects without specific recitation.

### DETAILED DESCRIPTION

Many medical treatments are known to impact or be impacted by kidney function, however, there may be desire to continue use of such medical treatments. Improved understanding of how these medical treatments affect and are affected by kidney function may result in increased treatment efficacy and reduction of harm to kidneys and other body systems.

Dialysis is a treatment for kidney failure that rids the body of unwanted toxins, waste products, and excess fluids by filtering a patient's blood. Dialysis helps to keep the healthy balance of water, salts, and minerals in a body. Dialysis also helps control blood pressure. Dialysis treatment may be performed by a hemodialysis machine or a peritoneal dialysis machine. During hemodialysis, blood is filtered through a dialyzer (i.e., dialysis machine). Blood and dialysate (e.g., dialysis solution) pass, allowing waste products to move out of the blood and into the dialysate in the dialysis machine and be discarded. During peritoneal dialysis, a catheter is placed in a patient's peritoneal cavity and dialysate is used to filter the blood through the peritoneal membrane. Waste products move out of the blood into the dialysate which is eventually removed and discarded. Peritoneal dialysis is often performed at home and at night while a patient is sleeping.

Diuretics are medications used to treat high blood pressure by lowering fluid volume in the body, and thus reducing blood pressure. Diuretics function by increasing the amount of fluid passed from the body in urine. Diuretics may be non-potassium sparing or potassium-sparing. Non-potassium sparing diuretics (e.g., thiazide and loop) function without regard for the amount of potassium lost from the body in the urine. Potassium sparing diuretics increase the amount of fluid removed from the body but do not reduce potassium levels. Diuretics have different pharmacokinetic availability for different patients and each may act different on each patient.

As shown with diuretics and dialysis, medical treatments may have different pharmacokinetic activity and biological responses specific to each patient. The effective period of a medical treatment may be one or more periods of time during which a medical treatment induces a biological response in a user. A biological response may include activity, absorption, pharmacodynamics, affinity, and/or efficacy of a medical treatment on a patient.

Thus, it is desirable to tailor medical treatments to the activity and biological responses of a specific patient. Further, tailoring of medical treatments may include adjustments during a treatment to improve activity and biological responses.

Overall, existing methods for determining treatment parameters suffer from a first problem of failing to consider an individual patient's biological response and treatment pharmacokinetic activity, including a patient's individual kidney function. Currently, using existing methods, treatment parameters may never be adjusted to a patient's response or only adjusted gradually over time based on a patient and/or health care provider (HCP) feedback. Furthermore, using existing methods, treatment parameters may not be determined using patient-specific data.

Existing methods for determining treatment parameters also suffer from a second problem of failing to account for an individual patient's current and changing health status. In particular, existing methods fail to continuously monitor the health of a patient by monitoring the concentration of changing analytes, such as potassium and/or glucose to indicate a patient's current and evolving state. As used herein, the term “continuous” may mean fully continuous, semi-continuous, periodic, etc. Such continuous monitoring of analytes is advantageous in diagnosing and staging a disease because the continuous measurements provide continuously up to date measurements as well as information on the trend and rate of analyte change over a continuous period. Such information may be used to predict analyte patterns prior to, during, and post treatment, determine likelihood of an adverse event during and subsequent to a treatment, generate optimal treatment parameters and/or other recommendations for the pre-, during, and post-treatment periods etc.

As a result of these problems, currently, medical treatments are not optimized for the health of a patient with kidney disease and/or diabetes and not adjusted to complement a patient's current health status. However, predicting a patient's kidney function associated with a medical treatment, determining likelihood of an adverse event during a treatment, and generating optimized treatment parameters may reduce the risk of adverse health events and prevent deterioration of overall kidney function. Such optimized parameters may account for competing risks and promote overall health of a patient, including reduction of risk of serious medical conditions and even death.

Accordingly, certain embodiments described herein provide a technical solution to the technical problems described above by providing an improved decision support and diagnostic system that is configured to account for the effects of medical treatments on patient's physiology (e.g., analyte levels) and the impact of patient physiology (e.g., reduced kidney function) on efficacy of medical treatments in order to optimize treatment for a patient to reduce risk of adverse health events. As discussed in more detail herein, the decision support system presented herein is designed to provide optimal treatment parameters for medical treatments that can affect be affected by patient physiology, as well as other decision support for management of such medical treatments.

For example, a decision support system described herein is configured to collect and/or generate data including for example, analyte data, patient information, and non-analyte sensor data during various time periods (e.g., a treatment period, pre-treatment period, and/or post-treatment period), to create various corresponding physiological profiles that can be used to (1) identify risk of adverse events during the various time periods based on the corresponding physiological profiles, (2) make patient-specific treatment decisions or recommendations to help address the identified risk of adverse events, including providing recommended treatment parameters for administration of medical treatments and/or automatically controlling the operations of one or more medical devices (e.g., dialysis machine, insulin pump, etc.) based on such recommended treatment parameters. Additionally or alternatively, the continuous analyte monitoring system may provide decision support to a patient based on a variety of collected data, including analyte data, patient information, secondary sensor data (e.g., non-analyte data), etc. For example, the analyte data may include continuously monitored glucose data and/or continuously monitored potassium data in addition to other continuously monitored analyte data, such as lactate, insulin, phosphate, bicarbonate, calcium, magnesium, sodium, blood urea nitrogen (BUN) data, and/or other data relating to other analytes mentioned herein. The collected data also includes patient information, which may include information related to age, gender, kidney disease, family history of kidney disease, other health conditions, etc. Secondary sensor data may include accelerometer data, heart rate data (ECG, HRV, HR, etc.), temperature, blood pressure, sweat sensor, impedance sensor, dialysis machine data, or any other sensor data other than analyte data.

Additionally or alternatively, the decision support system described herein may use various algorithms or artificial intelligence (AI) models, such as machine-learning models, trained based on patient-specific data and/or population data to provide real-time decision support to a patient based on the collected information about the patient. For example, certain aspects are directed to algorithms and/or machine-learning models designed to provide decision support, including predicting and providing optimal treatment parameters for a treatment (e.g., based on historical and/or rea-time data indicative of the impact of the treatment on patient physiology), predicting and alerting the patient about the likelihood of averse health events associated with a treatment, recommend health related actions to reduce the likelihood of the adverse health events, automatically control operations of a medical device (e.g., dialysis machine) based on the predicted optimal treatment parameters, or any combination thereof. The algorithms and/or machine-learning models may be used in combination with one or more continuous analyte sensors, including at least a continuous glucose sensor or a continuous potassium sensor, to provide real-time diabetes assessment.

The algorithms and/or machine-learning models may take into consideration population data, personalized patient-specific data, or a combination of both when determining a likelihood of an adverse event, providing decision support (e.g., optimized treatment parameters) for medical treatments, and some of the other outputs described herein. Additionally or alternatively, algorithms and/or machine-learning models may take into account physiological profiles created for patients based on monitoring the patients throughout various time periods (e.g., a treatment period, pre-treatment period, and/or post-treatment period)

According to certain embodiments, prior to deployment, the machine learning models are trained with training data, e.g., including user-specific data and/or population data. As described in more detail herein, the population data may be provided in a form of a dataset including data records of historical patients with varying stages of kidney disease, varying types of other comorbidities and with a history of various medical treatments. Each data record may be used as input into the machine learning models to optimize such models to generate accurate predictions around likelihood of adverse events during various time periods as well as decision support output (e.g., optimal treatment parameters, recommendations, etc.). The combination of a continuous analyte monitoring system with machine learning models and/or algorithms for (1) predicting the effect of medical treatment on patient physiology, including predicting likelihood of adverse events occurring during various time periods (e.g., a treatment period, pre-treatment period, and/or post-treatment period) and (2) providing decision support (e.g., predicting optimal treatment parameters, providing recommendations, etc.) for the management of treatments for kidney disease patients. For example, the decision support system may be used to improve efficacy of medical treatments, reduce the likelihood of adverse events during and after a medical treatment (e.g., dialysis), reduce unnecessary medical treatment, prevent new or worsening kidney dysfunction, and/or improve kidney function. Improved medical treatment reduces risk of hospitalization, complications, and death, in some cases.

Through the combination of a continuous analyte monitoring system with machine learning models and/or algorithms, the decision support system described herein is configured to provide the necessary accuracy and reliability patients expect. For example, biases, human errors, and emotional influence may be minimized when assessing the determining likelihood of an adverse event during various time periods (e.g., during or after a treatment period), and/or generating decision support outputs (e.g., optimal treatment parameters). Further, machine learning models and algorithms in combination with analyte monitoring systems may provide insight into patterns and/or trends of decreasing health of a patient, at least with respect to the kidney, which may have been previously missed. Accordingly, the decision support system described herein improves existing decision support systems and, more generally, the field of disease monitoring, diagnosis, and treatment.

### Example Decision Support System Including an Example Analyte Sensor

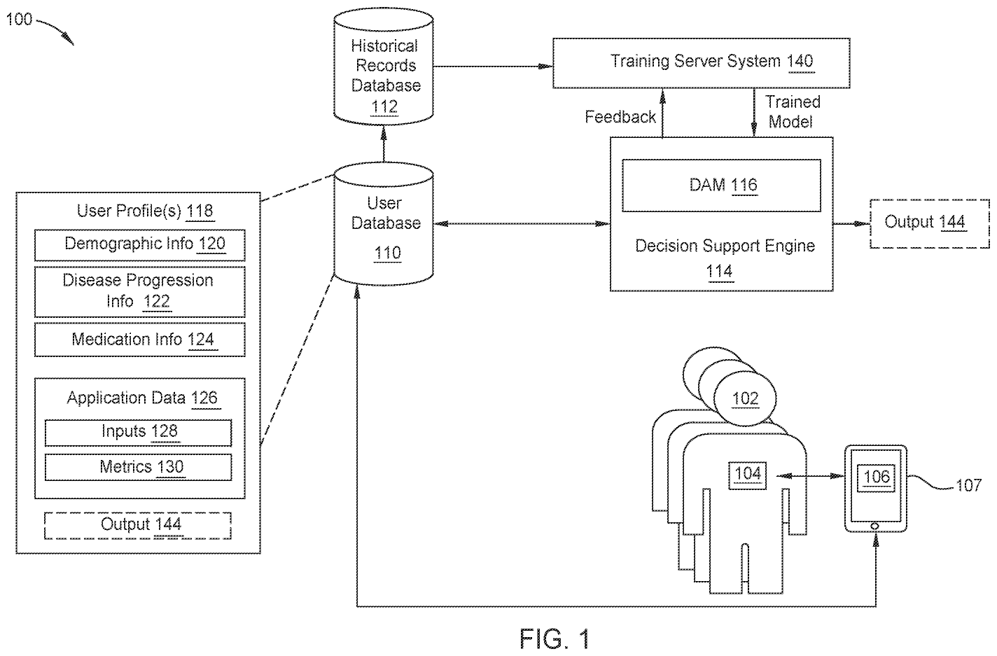

FIG. 1 illustrates an example decision support system 100 for predicting the effect of medical treatment on patient physiology, determining likelihood of an adverse event during a treatment period, a pre-treatment period, and/or a post-treatment period, and/or generating optimal treatment parameters and/or other recommendations. Decision support system 100 is configured to provide decision support to users 102 (individually referred to herein as a user and collectively referred to herein as users), using a continuous analyte monitoring system 104, including, at least, a continuous analyte sensor. A user, additionally or alternatively, may be the patient or, in some cases, the patient's caregiver. Additionally or alternatively, decision support system 100 includes continuous analyte monitoring system 104, a display device 107 that executes application 106, a decision support engine 114, a user database 110, a historical records database 112, a training system 140, and a decision support engine 114, each of which is described in more detail below.

The term “analyte” as used herein is a broad term used in its ordinary sense, including, without limitation, to refer to a substance or chemical constituent in a biological fluid (for example, blood, interstitial fluid, cerebral spinal fluid, lymph fluid or urine) that can be analyzed. Analytes can include naturally occurring substances, artificial substances, metabolites, and/or reaction products. Analytes for measurement by the devices and methods may include, but may not be limited to, potassium, glucose, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); androstenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; cystatin C d-penicillamine; de-ethylchloroquine; dehydroepiandrosterone sulfate; DNA (acetylator polymorphism, alcohol dehydrogenase, alpha 1-antitrypsin, glucose-6-phosphate dehydrogenase, hemoglobin A, hemoglobin S, hemoglobin C, hemoglobin D, hemoglobin E, hemoglobin F, D-Punjab, hepatitis B virus, HCMV, HIV-1, HTLV-1, MCAD, RNA, PKU, Plasmodium vivax, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione peroxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sisomicin; somatomedin C; specific antibodies recognizing any one or more of the following that may include (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, coronavirus including but not limited to Covid-19, Rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain implementations. Ions are a charged atom or compounds that may include the following (sodium, potassium, calcium, chloride, nitrogen, or bicarbonate, for example). The analyte can be naturally present in the biological fluid, for example, a metabolic product, a hormone, an antigen, an antibody, an ion and the like. Alternatively, the analyte can be introduced into the body or exogenously, for example, a contrast agent for imaging, a radioisotope, a chemical agent, a fluorocarbon-based synthetic blood, a challenge agent analyte (e.g., introduced for the purpose of measuring the increase and or decrease in rate of change in concentration of the challenge agent analyte or other analytes in response to the introduced challenge agent analyte), or a drug or pharmaceutical composition, including but not limited to exogenous insulin; glucagon, ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA), and intermediaries in the Citric Acid Cycle.

While the analytes that are measured and analyzed by the devices and methods described herein include lactate, insulin, phosphate, bicarbonate, calcium, magnesium, sodium, blood urea nitrogen (BUN), and other analytes listed, but not limited to, above may also be considered and measured by, for example, analyte monitoring system 104.

Additionally or alternatively, continuous analyte monitoring system 104 is configured to continuously measure one or more analytes and transmit the analyte measurements to an electric medical records (EMR) system (not shown in FIG. 1). An EMR system is a software platform which allows for the electronic entry, storage, and maintenance of digital medical data. An EMR system is generally used throughout hospitals and/or other caregiver facilities to document clinical information on patients over long periods. EMR systems organize and present data in ways that assist clinicians with, for example, interpreting health conditions and providing ongoing care, scheduling, billing, and follow up. Data contained in an EMR system may also be used to create reports for clinical care and/or disease management for a patient. Additionally or alternatively, the EMR may be in communication with decision support engine 114 (e.g., via a network) for performing the techniques described herein. In particular, as described herein, decision support engine 114 may obtain data associated with a user, use the obtained data as input into one or more trained model(s), and output a prediction. In some cases, the EMR may provide the data to decision support engine 114 to be used as input into the one or more models. Further, in some cases, decision support engine 114, after making a prediction, may provide the output prediction to the EMR. In other embodiments intermediary systems such as interface engines may be used with or without patient matching algorithms, systems, or master patient index to coordinate data between such systems, analyte monitoring systems, a cloud database and or the EMR.

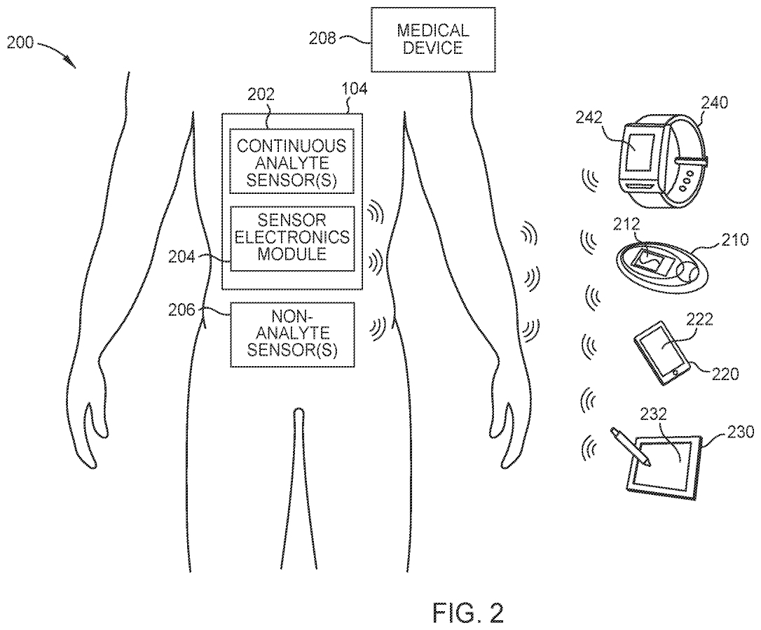

Additionally or alternatively, continuous analyte monitoring system 104 is configured to continuously measure one or more analytes and transmit the analyte measurements to display device 107 for use by application 106. In some embodiments, continuous analyte monitoring system 104 transmits the analyte measurements to display device 107 through a wireless connection (e.g., Bluetooth connection). Additionally or alternatively, display device 107 is a smart phone. However, in certain other embodiments, display device 107 may instead be any other type of computing device such as a laptop computer, a smart watch, a tablet, or any other computing device capable of executing application 106. In some embodiments, continuous analyte monitoring system 104 and/or analyte sensor application 106 transmit the analyte measurements to one or more other individuals having an interest in the health of the patient (e.g., a family member or physician for real-time treatment and care of the patient). Continuous analyte monitoring system 104 may be described in more detail with respect to FIG. 2.

Application 106 is a mobile health application that is configured to receive and analyze analyte measurements from analyte monitoring system 104. In particular, application 106 stores information about a user, including the user's analyte measurements, in a user profile 118 associated with the user for processing and analysis, as well as for use by decision support engine 114 to provide decision support recommendations or guidance to the user.

Decision support engine 114 refers to a set of software instructions with one or more software modules, including data analysis module (DAM) 116. Additionally or alternatively, decision support engine 114 executes entirely on one or more computing devices in a private or a public cloud. In such embodiments, application 106 communicates with decision support engine 114 over a network (e.g., Internet). In some other embodiments, decision support engine 114 executes partially on one or more local devices, such as display device 107, and partially on one or more computing devices in a private or a public cloud. In some other embodiments, decision support engine 114 executes entirely on one or more local devices, such as display device 107. As discussed in more detail herein, decision support engine 114 may provide decision support recommendations to the user via application 106. Decision support engine 114 provides decision support recommendations based on information included in user profile 118.

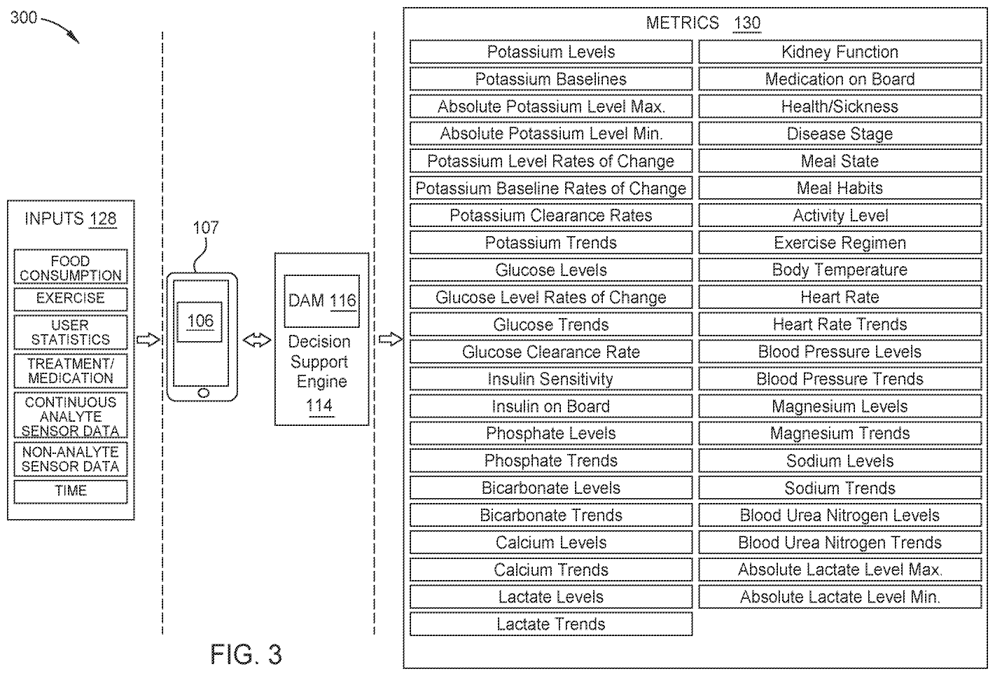

User profile 118 may include information collected about the user from application 106. For example, application 106 provides a set of inputs 128, including the analyte measurements received from continuous analyte monitoring system 104, that are stored in user profile 118. Additionally or alternatively, inputs 128 provided by application 106 include other data in addition to analyte measurements received from continuous analyte monitoring system 104. For example, application 106 may obtain additional inputs 128 through manual user input, one or more other non-analyte sensors or devices, other applications executing on display device 107, etc. Non-analyte sensors and devices include one or more of, but are not limited to, an insulin pump, an electrocardiogram (ECG) sensor or heart rate monitor, an acoustic sensor, a blood pressure sensor, atmospheric pressure sensor, atmospheric oxygen sensor, a sweat sensor, a respiratory sensor, a thermometer, a peritoneal dialysis machine, a hemodialysis machine, sensors or devices provided by display device 107 (e.g., accelerometer, camera, global positioning system (GPS), heart rate monitor, thermometer, etc.) or other user accessories (e.g., a smart watch), or any other sensors or devices that provide relevant information about the user. In certain embodiments, non-analyte sensors may be incorporated in the display device 107 or may include separate sensors and/or devices not incorporated into the display device 107. Inputs 128 of user profile 118 provided by application 106 are described in further detail below with respect to FIG. 3.

DAM 116 of decision support engine 114 is configured to process the set of inputs 128 to determine one or more metrics 130. Metrics 130, discussed in more detail below with respect to FIG. 3, may, at least in some cases, be generally indicative of the health or state of a user, such as one or more of the user's physiological state, trends associated with the health or state of a user, etc. In certain embodiments, the user's physiological state may be based on the user's core body temperature, blood pressure, heart rate, circadian rhythms, etc. Additionally or alternatively, metrics 130 may then be used by decision support engine 114 as input for providing guidance to a user. As shown, metrics 130 are also stored in user profile 118.

User profile 118 also includes demographic info 120, disease progression info 122, and/or medication info 124. Additionally or alternatively, such information may be provided through user input or obtained from certain data stores (e.g., electronic medical records (EMRs), etc.). Additionally or alternatively, demographic info 120 may include one or more of the user's age, body mass index (BMI), ethnicity, gender, etc. Additionally or alternatively, disease progression info 122 may include information about a disease of a user, such as whether the user has been previously diagnosed with acute kidney injury (AKI), chronic kidney disease (CKD), and/or diabetes, or have had a history of hyperkalemia, hypokalemia, hyperglycemia, hypoglycemia, etc. Additionally or alternatively, information about a user's disease may also include the length of time since diagnosis, the stage of disease, the level of disease control, level of compliance with disease management therapy, predicted kidney function, other types of diagnosis (e.g., heart disease, obesity, etc.) or measures of health (e.g., heart rate, blood pressure, exercise, stress, sleep, etc.), and/or the like. Additionally or alternatively, disease progression info 122 may be provided as an output of one or more predictive algorithms and/or trained models based on analyte sensor data generated, for example, through continuous analyte monitoring system 104. Additionally or alternatively, disease progression info 122 may be provided through manual or semi-manual input from a clinical provider. For example, disease progression info 122 may be provided as an output of one or more models, and the output may then be confirmed by a clinical provider.

Additionally or alternatively, medication info 124 may include information about the amount, frequency, and type of a medication treatment (e.g., medication and/or health treatment) administered to a user. Additionally or alternatively, the amount, frequency, and type of a medical treatment administered to a user is time-stamped and correlated with the user's time-stamps analyte levels, analyte rates of change, adverse events, indications of kidney function, etc., thereby, indicating the impact the amount, frequency, and type of the medical treatment had on the user's analyte levels, kidney function, risk of experiencing adverse events, etc.

Additionally or alternatively, medication information 124 may include information about one or more medical treatments known to be helpful in managing kidney function. One or more medical treatments to control in relation to managing kidney function may include dialysis, including hemodialysis and/or peritoneal dialysis, and the like. As described in more detail below decision support system 100 may be configured to use medication information 124 to determine optimal medical treatment parameters to be prescribed to different users. In particular, decision support system 100 may be configured to identify one or more optimal dialysis treatment parameters based on the health of the patient, the patient's current condition, and/or effectiveness of dialysis treatment. Additionally or alternatively, decision support system 100 may be configured to identify when a user is a good candidate for a kidney transplant, including prioritizing a user from a group of users for a kidney transplant. Transplant information may further include additional prognostic information, such as the optimal time to initiate a kidney transplant for a particular user.

Additionally or alternatively, medication information 124 may include information about consumption of one or more drugs known to damage the kidney. One or more drugs known to damage the kidney may include nonsteroidal anti-inflammatory drugs (NSAIDS) such as ibuprofen (e.g., Advil, Motrin) and naproxen (e.g., Aleve), vancomycin, iodinated radiocontrast (e.g., refers to any contrast dyes used in diagnostic testing), angiotensin-converting enzyme (ACE) such as lisinopril, enalapril, and ramipril, aminoglycoside antibiotics such as neomycin, gentamicin, tobramycin, and amikacin, antiviral human immunodeficiency virus (HIV) medications, zoledronic acid (e.g., Zometa, Reclast), foscarnet, and the like.

Additionally or alternatively, medication information 124 may include information about consumption of one or more drugs known to control the complications of kidney disease. One or more drugs known to control the complications of kidney disease may include medications to lower blood pressure and preserve kidney function such as ACE inhibitors or angiotensin II receptor blockers, medications to treat anemia such as supplements of the hormone erythropoietin, medications used to lower cholesterol levels such as statins, medications used to prevent weak bones such as calcium and vitamin D supplements, phosphate binders, and the like.

Additionally or alternatively, medication information 124 may include information about consumption of one or more drugs or treatments known to control and/or improve glucose homeostasis. One or more drugs known to control and/or improve glucose homeostasis may include medications to lower blood glucose levels such as insulin, including rapid acting, and long-acting insulin, other glycemic controlling medications, such as metformin, and the like.

Additionally or alternatively, medication information 124 may include information about consumption of one or more drugs or treatments known to cause hypoglycemia and/or hyperglycemia. One or more medications known to cause hypoglycemia may include ACE inhibitors, beta blockers, pentamidine, quinolone antibiotics, and salicylates. Alternatively, one or more medications known to cause hyperglycemia, including increased heart rate and elevated systolic blood pressure, may include fluoroquinolone antibiotics, beta blockers, thiazide and thiazide-like diuretics, second-generation antipsychotics (SGAs), corticosteroids, calcineurin inhibitors (CNIs), and protease inhibitors.

Additionally or alternatively, user profile 118 is dynamic because at least part of the information that is stored in user profile 118 may be revised over time and/or new information may be added to user profile 118 by decision support engine 114 and/or application 106. Accordingly, information in user profile 118 stored in user database 110 provides an up-to-date repository of information related to a user.

User database 110, in some embodiments, refers to a storage server that operates in a public or private cloud. User database 110 may be implemented as any type of datastore, such as relational databases, non-relational databases, key-value datastores, file systems including hierarchical file systems, and the like. In some exemplary implementations, user database 110 is distributed. For example, user database 110 may comprise a plurality of persistent storage devices, which are distributed. Furthermore, user database 110 may be replicated so that the storage devices are geographically dispersed.

User database 110 includes user profiles 118 associated with a plurality of users who similarly interact with application 106 executing on the display devices 107 of the other users. User profiles stored in user database 110 are accessible to not only application 106, but decision support engine 114, as well. User profiles in user database 110 may be accessible to application 106 and decision support engine 114 over one or more networks (not shown). As described above, decision support engine 114, and more specifically DAM 116 of decision support engine 114, can fetch inputs 128 from user database 110 and compute a plurality of metrics 130 which can then be stored as application data 126 in user profile 118.

Additionally or alternatively, user profiles 118 stored in user database 110 may also be stored in historical records database 112. User profiles 118 stored in historical records database 112 may provide a repository of up-to-date information and historical information for each user of application 106. Thus, historical records database 112 essentially provides all data related to each user of application 106, where data is stored according to an associated timestamp. The timestamp associated with each piece of information stored in historical records database 112 may identify, for example, when information related to a user has been obtained and/or updated.

Further, historical records database 112 may maintain time series data collected for users over a period of time, including for users who use continuous analyte monitoring system 104 and application 106. For example, analyte data for a user who has used continuous analyte monitoring system 104 and application 106 for a period of five years to manage the user's health may have time series analyte data associated with the user maintained over the five-year period.

Further, additionally or alternatively, historical records database 112 may include data for one or more patients who are not users of continuous analyte monitoring system 104 and/or application 106. In addition, historical records database 112 may include information (e.g., user profile(s)) related to one or more patients examined by, for example, a healthcare physician (or other known method), and not prescribed medical treatments associated with kidney function, as well as information (e.g., user profile(s)) related to one or more patients who were examined by, for example, a healthcare physician (or other known method) and were previously prescribed medical treatments associated with kidney function. Data stored in historical records database 112 may be referred to herein as population data.

Data related to each patient stored in historical records database 112 may provide time series data collected over the disease lifetime of the patient, wherein the disease may be kidney disease. For example, the data may include information about the patient prior to being diagnosed with kidney disease and information associated with the patient during the lifetime of the disease, including information related to each stage of kidney disease as it progressed and/or regressed in the patient. The data may additionally, or alternatively, include information related to other diseases, such as hyperkalemia, hypokalemia, hyperglycemia, hypoglycemia, diabetes, hypertension, heart conditions and diseases (e.g., coronary artery disease, peripheral artery disease, arrhythmic diseases and conditions, etc.), or similar diseases that are co-morbid in relation to kidney disease. Such information may indicate symptoms of the patient, physiological states of the patient, potassium levels of the patient, glucose levels of the patient, lactate levels of the patient, insulin levels of the patient, phosphate levels of the patient, bicarbonate levels of the patient, calcium levels of the patient, magnesium levels of the patient, sodium levels of the patient, blood urea nitrogen levels of the patient, states/conditions of one or more organs of the patient, habits of the patient (e.g., activity levels, food consumption, etc.), medical treatments prescribed throughout the lifetime of kidney disease. The data may further include information about the patient's kidney function and occurrence of adverse events prior, during, and after effective periods of various treatments.

For a patient who is also diabetic, the data may include information about the patient prior to being diagnosed with diabetes and information associated with the patient during the lifetime of the disease, including information related to diabetes as it progressed and/or regressed in the patient. The data may additionally, or alternatively, include information related to other diseases, such as kidney disease, hyperglycemia, hypoglycemia, hypertension, heart conditions and diseases, or similar diseases that are co-morbid in relation to diabetes. Such information may indicate symptoms of the patient, physiological states of the patient, glucose levels of the patient, potassium levels of the patient, lactate levels of the patient, insulin levels of the patient, states/conditions of one or more organs of the patient, habits of the patient (e.g., activity levels, food consumption, etc.), medical treatments prescribed, medical treatment adherence, etc., throughout the lifetime of the disease. The data may further include information about the patient's diabetic state and occurrence of adverse events prior, during, and after effective periods of various treatments.

Although depicted as separate databases for conceptual clarity, in some embodiments, user database 110 and historical records database 112 may operate as a single database. That is, historical and current data related to users of continuous analyte monitoring system 104 and application 106, as well as historical data related to patients that were not previously users of continuous analyte monitoring system 104 and application 106, may be stored in a single database. The single database may be a storage server that operates in a public or private cloud.

As mentioned previously, decision support system 100 is configured to predict the effect of medical treatments on kidney function and provide decision support for the management of medical treatments for patients with kidney disease using continuous analyte monitoring system 104, including, at least, one of a continuous glucose sensor and a continuous potassium sensor. For example, decision support engine 114 may be configured to collect and/or generate data including for example, analyte data, patient information, and non-analyte sensor data during various time periods (e.g., a treatment period, pre-treatment period, and/or post-treatment period), to create various corresponding physiological profiles that can be used to (1) identify risk of adverse events during the various time periods based on the corresponding physiological profiles, (2) make patient-specific treatment decisions or recommendations to help address the identified risk of adverse events, including providing recommended treatment parameters for administration of medical treatments and/or automatically controlling the operations of one or more medical devices (e.g., dialysis machine, insulin pump, etc.) based on such recommended treatment parameters. Further, additionally or alternatively, each user's metrics recorded over time may be analyzed to provide an indication of the improvement or the deterioration of the patient's health.

Additionally or alternatively, decision support engine 114 may be used to collect information associated with a user in user profile 118 to perform analytics thereon for predicting the effect of medical treatment on patient physiology and providing one or more recommendations for the management of medical treatments affecting kidney function. For example, decision support engine 114 may perform analytics on collected information associated with a user in user profile 118 to determine analyte rate(s) of change during various time periods (e.g., a treatment period, pre-treatment period, and/or post-treatment period) and generate corresponding physiological profiles. Additionally or alternatively, based on the generated physiological profiles, decision support engine 114 may determine the likelihood a user will experience an adverse health event during corresponding time periods and generate optimal treatment parameters or decision support recommendations to help reduce the likelihood.

User profile 118 may be accessible to decision support engine 114 over one or more networks (not shown) for performing such analytics. Additionally or alternatively, decision support engine 114 is configured to provide real-time and/or non-real-time decision support around kidney function to the user and/or others, including but not limited, to healthcare providers (HCP), family members of the user, caregivers of the user, researchers, and/or other individuals, systems, and/or groups supporting care or learning from the data.

Additionally or alternatively, decision support engine 114 may utilize one or more trained machine learning models for (1) identifying risk of adverse events during the various time periods based on the corresponding physiological profiles, (2) making patient-specific treatment decisions or recommendations to help address the identified risk of adverse events, including providing recommended treatment parameters for administration of medical treatments and/or automatically controlling the operations of one or more medical devices (e.g., dialysis machine, insulin pump, etc.) based on such recommended treatment parameters. In the illustrated embodiment of FIG. 1, decision support engine 114 may utilize trained machine learning model(s) provided by a training system 140. Although depicted as a separate server for conceptual clarity, in some embodiments, training system 140 and decision support engine 114 may operate as a single server. That is, the model may be trained and used by a single server, or may be trained by one or more servers and deployed for use on one or more other servers. Additionally or alternatively, the model may be trained on one or many virtual machines (VMs) running, at least partially, on one or many physical servers in relational and/or non-relational database formats.

Training system 140 is configured to train the machine learning model(s) using training data, which may include data (e.g., from user profiles) associated with one or more patients (e.g., users or non-users of continuous analyte monitoring system 104 and/or application 106) (1) without kidney disease and not prescribed medical treatments affecting kidney function, (2) without kidney disease, but prescribed medical treatments affecting kidney function, (3) with kidney disease but not prescribed medication and medical treatments affecting kidney function, or (4) with kidney disease and prescribed medical treatments affecting kidney function. The training data may be stored in historical records database 112 and may be accessible to training system 140 over one or more networks (not shown) for training the machine learning model(s). The training data may also, in some cases, include user-specific data for a user over time.

The training data refers to a dataset that, for example, has been featurized and labeled. For example, the dataset may include a plurality of data records, each including information corresponding to a different user profile stored in user database 110, where each data record is featurized and labeled. In machine learning and pattern recognition, a feature is an individual measurable property or characteristic. Generally, the features that best characterize the patterns in the data are selected to create predictive machine learning models. Data labeling is the process of adding one or more meaningful and informative labels to provide context to the data for learning by the machine learning model.

As an illustrative example, each relevant characteristic of a user, which is reflected in a corresponding data record, may be a feature used in training the machine learning model. Such features may include the user's demographic features (e.g., age, gender, etc.), user's physiological features, etc. The user's physiological features may include glucose levels; change (e.g., delta) in glucose levels from a first timestamp to a second timestamp; glucose levels over time (e.g., glucose levels from two or more subsequent timestamps); glucose clearance rate; change (e.g., delta) in glucose clearance rate from a first time stamp to one or more subsequent timestamps; glucose clearance rate over time (e.g., glucose clearance rate from two or more subsequent timestamps; mean glucose levels over time (e.g., day to day, week to week, month to month, etc.) (e.g., mean glucose from two or more subsequent timestamps); mean glucose levels over the course of a first day compared to mean glucose levels over the course of a second day (e.g., mean glucose levels from the morning, afternoon, or evening, for example); mean glucose levels during event specific time ranges on a first day (e.g., morning, before going to sleep, during sleep, post-exercise, post-dialysis) compared to mean glucose levels during event specific time ranges on a second day; glycemic variability (e.g., standard deviation of mean glucose); change (e.g., delta) in glycemic variability from a first series of timestamps; (e.g., glycemic variability from a first timestamp to one or more subsequent time stamps) to a second series of timestamps (e.g., glycemic variability from a second timestamp to one or more subsequent timestamps); glycemic variability over time (e.g., glycemic variability from two or more subsequent timestamps); time in range (TIR) (e.g., glucose levels at, above, below, or between a threshold); change (e.g., delta) in TIR from a first timestamp to a second timestamp; TIR over time (e.g., TIR from two or more subsequent timestamps); glucose clearance rate; change (e.g., delta) in glucose clearance rate from a first time stamp to one or more subsequent timestamps; glucose clearance rate over time (e.g., blood and/or kidney glucose clearance rate from two or more subsequent timestamps; diabetes presence and/or severity; change (e.g., delta) in diabetes stage or severity from a first timestamp to a second timestamp; the derivative of the measured linear system of glucose level at one or more specific timestamps and/or the difference in derivatives to determine rates of change in the slope of the increase or decrease in glucose levels; the derivative of the determined linear system of glucose clearance rate at a specific timestamp, or a specific series of timestamps, and/or the difference in derivatives to determine rates of change in the slope of the increase or decrease in glucose clearance rate; insulin level; change (e.g., delta) in insulin levels from a first timestamp to a second timestamp; insulin levels over time (e.g., insulin levels from two or more subsequent timestamps); the derivative of the measured linear system of insulin level at one or more specific timestamps and/or the difference in derivatives to determine rates of change in the slope of the increase or decrease in insulin levels etc.

The user's other physiological features may include the user's potassium levels; change (e.g., delta) in potassium levels from a first timestamp to a second timestamp; potassium levels over time (e.g., potassium levels from two or more subsequent timestamps); potassium clearance rate; change (e.g., delta) in potassium clearance rate from a first time stamp to one or more subsequent timestamps; potassium clearance rate over time (e.g., potassium clearance rate from two or more subsequent timestamps; diabetes presence and/or severity; change (e.g., delta) in diabetes stage or severity from a first timestamp to a second timestamp; the derivative of the measured linear system of potassium level at one or more specific timestamps and/or the difference in derivatives to determine rates of change in the slope of the increase or decrease in potassium levels; the derivative of the determined linear system of potassium clearance rate at a specific timestamp, or a specific series of timestamps, and/or the difference in derivatives to determine rates of change in the slope of the increase or decrease in potassium clearance rate; insulin level; change (e.g., delta) in insulin levels from a first timestamp to a second timestamp; insulin levels over time (e.g., insulin levels from two or more subsequent timestamps); the derivative of the measured linear system of insulin level at one or more specific timestamps and/or the difference in derivatives to determine rates of change in the slope of the increase or decrease in insulin levels etc.

Yet, other features may relate to the patient's kidney function, such as the presence and/or severity of kidney disease; change (e.g., delta) in kidney disease stage or severity from a first timestamp to a second timestamp; kidney function; change (e.g., delta) in kidney function from a first timestamp to a second timestamp; kidney function over time (e.g., kidney function from two or more subsequent timestamps); rate of change in kidney function over time; kidney function before, during, and after a medical treatment; change (e.g., delta) in kidney function from a first timestamp to a second timestamp, where the first and the second time stamps may each be before, during, or after a medical treatment; kidney function over time (e.g., kidney function from two or more subsequent timestamps before, during, and after a medical treatment); rate of change in kidney function over time (e.g., based on two or more subsequent timestamps before, during, and after a medical treatment).

Additional or alternative features may include the user's lactate level; change (e.g., delta) in lactate levels from a first timestamp to a second timestamp; lactate levels over time (e.g., lactate levels from two or more subsequent timestamps); the derivative of the measured linear system of lactate level at one or more specific timestamps and/or the difference in derivatives to determine rates of change in the slope of the increase or decrease in lactate levels; phosphate level; change (e.g., delta) in phosphate levels from a first timestamp to a second timestamp; phosphate levels over time (e.g., phosphate levels from two or more subsequent timestamps); the derivative of the measured linear system of phosphate level at one or more specific timestamps and/or the difference in derivatives to determine rates of change in the slope of the increase or decrease in phosphate levels; bicarbonate level; change (e.g., delta) in bicarbonate levels from a first timestamp to a second timestamp; bicarbonate levels over time (e.g., bicarbonate levels from two or more subsequent timestamps); the derivative of the measured linear system of bicarbonate level at one or more specific timestamps and/or the difference in derivatives to determine rates of change in the slope of the increase or decrease in bicarbonate levels; calcium level; change (e.g., delta) in calcium levels from a first timestamp to a second timestamp; calcium levels over time (e.g., calcium levels from two or more subsequent timestamps); the derivative of the measured linear system of calcium level at one or more specific timestamps and/or the difference in derivatives to determine rates of change in the slope of the increase or decrease in calcium levels; magnesium level; change (e.g., delta) in magnesium levels from a first timestamp to a second timestamp; magnesium levels over time (e.g., magnesium levels from two or more subsequent timestamps); the derivative of the measured linear system of magnesium level at one or more specific timestamps and/or the difference in derivatives to determine rates of change in the slope of the increase or decrease in magnesium levels; sodium level; change (e.g., delta) in sodium levels from a first timestamp to a second timestamp; sodium levels over time (e.g., sodium levels from two or more subsequent timestamps); the derivative of the measured linear system of sodium level at one or more specific timestamps and/or the difference in derivatives to determine rates of change in the slope of the increase or decrease in sodium levels; blood urea nitrogen level; change (e.g., delta) in blood urea nitrogen levels from a first timestamp to a second timestamp; blood urea nitrogen levels over time (e.g., blood urea nitrogen levels from two or more subsequent timestamps); the derivative of the measured linear system of blood urea nitrogen level at one or more specific timestamps and/or the difference in derivatives to determine rates of change in the slope of the increase or decrease in blood urea nitrogen levels; blood pH level; change (e.g., delta) in blood pH levels from a first timestamp to a second timestamp; blood pH levels over time (e.g., blood urea nitrogen levels from two or more subsequent timestamps); the derivative of the measured linear system of blood pH level at one or more specific timestamps and/or the difference in derivatives to determine rates of change in the slope of the increase or decrease in blood pH levels.

In addition or alternatively, other features may include non-analyte data; change (e.g., delta) in non-analyte data from a first timestamp to a second timestamp; non-analyte data over time (e.g., non-analyte data from two or more subsequent timestamps); the derivative of the measured linear system of non-analyte data at one or more specific timestamps and/or the difference in derivatives to determine rates of change in the slope of the increase or decrease in non-analyte data; etc. Additionally or alternatively, non-analyte data may include ECG data, which is used and or correlated to potassium measurements. ECG data may be used with or without other combinations of inputs like glucose trends or potassium sensor trends. ECG is indicative of the body's physiologic response to the potassium levels which is most critical—some patients augment to tolerate higher or lower levels than normal without changes to ECG. Further, extracellular potassium concentration directly influences the depolarization and re-polarization of cardiac muscle. Therefore monitoring abnormalities in the ECG signal such as taller T waves, prolonged PR interval, smaller P wave and or widening of the QRS waves is advantageous. In scenarios where potassium levels are higher or lower than normal and these abnormalities are monitored and detected, alerts can be escalated for corrective action and/or medical intervention.

In addition or in an alternative, the dataset may include features associated with a patient's medical treatment and/or the impact of the medical treatment on the patient's physiology during and/or after the medical treatment. For example, such features may relate to medications, medical treatments, and/or health treatments; medical treatment parameters such as type, dosage, timing, frequency, composition, concentration, flow rate, volume, and/or other treatment parameters, including dialysis treatment parameters (e.g., hemolysis and/or peritoneal dialysis parameters); other one or more medication and/or treatments administered to the user, such as glycemic controlling medication, one or more drugs known to damage the kidney, one or more drugs known to control the complications of kidney disease that are prescribed to the user, and/or one or more medications for treating one or more symptoms of kidney disease, hyperkalemia, hypokalemia, diabetes, and/or other conditions and diseases the user may have. All of the medical treatment parameter features discussed above may be time-stamped so that a correlation between the impact of such parameters on a patient's physiology before, during, and/or after a corresponding medical treatment may be derived.

In addition or in the alternative, each data record in the dataset may be labeled with at least one of an indication as to a likelihood of the patient experiencing an adverse event before, during, and/or after a treatment period of a medical treatment, one or more treatment parameters for a medical treatment (e.g., dialysis), improvement or deterioration of kidney function during and/or after a medical treatment, and the effect of a change in treatment on the patient's physiology.

The model(s) are then trained by training system 140 using the featurized and labeled training data. In particular, the features of each data record may be used as input into the machine learning model(s), and the generated output may be compared to label(s) associated with the corresponding data record. The model(s) may compute a loss based on the difference between the generated output and the provided label(s). This loss is then used to modify the internal parameters or weights of the model. By iteratively processing each data record corresponding to each historical patient, additionally or alternatively, the model(s) may be iteratively refined to generate accurate predictions associated with the effects of medical treatments on patient physiology (e.g., kidney function, analyte levels, analyte rate of change, analyte clearance rate, etc.), risk of adverse health events before, during, and/or after a treatment period of a medical treatment, optimal treatment parameters for a medical treatment (e.g., to reduce the risk of adverse health events), improvement or deterioration of kidney function during and/or after a medical treatment etc. Further, in certain other embodiments, by iteratively processing each data record corresponding to each historical patient, additionally or alternatively, the model(s) may be iteratively refined to generate more accurate predictions.

As illustrated in FIG. 1, training system 140 deploys these trained model(s) to decision support engine 114 for use during runtime. For example, decision support engine 114 may obtain user profile 118 associated with a user, use information in user profile 118 as input into the trained model(s), and output a prediction. The prediction may be indicative of adverse risks associated with the medical treatment (e.g., shown as output 144 in FIG. 1). Output 144 generated by decision support engine 114 may also provide patient-specific treatment recommendations to reduce the likelihood of adverse events. For example, treatment recommendations may provide optimal treatment parameters to be administered. Providing optimal treatment parameters may also include automatically controlling the operations of one or more medical devices (e.g., dialysis machine, insulin pump, etc.) based on such optimal treatment parameters. Additionally or alternatively, treatment recommendations may include optimal medication dosing, kidney transplant prioritization, and/or additional testing to confirm appropriate treatment recommendations. Output 144 may be provided to the user (e.g., through application 106), the user's medical treatment device for automatically implementing the output 114, to a user's caretaker (e.g., a parent, a relative, a guardian, a teacher, a nurse, etc.), to a user's physician, or any other individual that has an interest in the wellbeing of the user for purposes of improving the user's health, such as, in some cases by effectuating the recommended treatment.

Additionally or alternatively, the user's own data is used to personalize the one or more models that are initially trained based on population data. For example, a model (e.g., trained using population data) may be deployed for use by decision support engine 114 to predict the risk of an adverse health event associated with a user's analyte trends and a current medical treatment. After making a prediction using the model, decision support engine 114 may be configured to obtain the user's actual physiological data (e.g., analyte levels, rates of change, analyte clearance rate, adverse events, etc.) and compute a loss between the prediction and the actual analyte data, which can be used for retraining the model. Accordingly, the model may continue to be retrained and personalized using the computed loss between the prediction and the actual physiological data to personalize the model for the user. In another example, a model (e.g., trained using population data) may be deployed for use by decision support engine 114 to predict the effect of a change in treatment (e.g., treatment parameters such as type, timing, dose, etc.) on a specific user experiencing an adverse health event in real-time. After making a prediction using the model, decision support engine 114 may be configured to obtain the user's actual occurrence, timing, severity of adverse events and compute a loss between the prediction and the actual occurrence, timing, severity of adverse events, which can be used for retraining the model. Accordingly, the model may continue to be retrained and personalized using the computed loss between the prediction and the actual physiological data as input into the model to personalize the model for the user. In another example, the personalization of the one or more models includes choosing a subset of population data from subjects with characteristics (e.g. demographics information, disease progression information, medication information) similar to those of the user. A model trained using the subset of population data may be deployed for use by decision support engine 114 to predict the risk of an adverse health event associated with a user's analyte trends and a current medical treatment.

Additionally or alternatively, output 144 generated by decision support engine 114 may be stored in user profile 118. Additionally or alternatively, output 144 may be a prediction as to the effect of medical treatment on patient physiology (e.g., kidney function, analyte levels, analyte rate of change, analyte clearance rate, etc.). Additionally or alternatively, output 144 may be patient-specific decisions or recommendations for medical treatment parameters to optimize the medical treatment. Additionally or alternatively, output 144 may be a prediction relating to analyte levels before, during, and/or after the treatment period of a medication. Additionally or alternatively, output 144 may be a prediction relating to a user's kidney function during the treatment period of a medication. Additionally or alternatively, output 144 may be a prediction of a user's risk for an adverse health event (e.g., hypoglycemia, hyperglycemia, hypokalemia, hyperkalemia, etc.) caused, for example, by a medical treatment. Additionally or alternatively, output 144 may be patient-specific optimized medical treatment parameters (e.g., dialysis treatment parameters). Output 144 stored in user profile 118 may be continuously updated by decision support engine 114. Accordingly, predictions and recommendations, originally stored as outputs 144 in user profile 118 in user database 110 and then passed to historical records database 112, may provide an indication of the progression of kidney disease associated with a medical treatment over time, as well as an indication as to the effectiveness of different medical treatment parameters associated with reduced risk of adverse health events.

Additionally or alternatively, the model may be trained to provide lifestyle recommendations, exercise recommendations, diet recommendations, medical treatment recommendations, medical intervention recommendations, and other types of decision support recommendations to help the user manage medical treatments based on the user's historical data, including how different treatment parameters, medication, food, and exercise have impacted the user's physiology in the past. Additionally or alternatively, real-time access to geographically local food and menu databases may inform recommendations for specific menu items to include or avoid depending on current glucose and potassium or other analyte information. Additionally or alternatively, the model may be trained to detect the underlying cause of certain improvements or deteriorations in the patient's physiology (e.g., occurrence of adverse events). For example, application 106 may display a user interface with a graph that shows the patient's analyte data or a score thereof with trend lines and indicate, e.g., retrospectively, what caused adverse events (e.g., different treatment parameters, food consumption, exercise, other medical treatments, declining kidney function, etc.).

FIG. 2 is a diagram 200 conceptually illustrating an example continuous analyte monitoring system 104 including example continuous analyte sensor(s) with sensor electronics, in accordance with certain aspects of the present disclosure. For example, system 104 may be configured to continuously monitor one or more analytes of a user, in accordance with certain aspects of the present disclosure.

Continuous analyte monitoring system 104 in the illustrated embodiment includes sensor electronics module 204 and one or more continuous analyte sensor(s) 202 (individually referred to herein as continuous analyte sensor 202 and collectively referred to herein as continuous analyte sensors 202) associated with sensor electronics module 204. Sensor electronics module 204 may be in wireless communication (e.g., directly or indirectly) with one or more of display devices 210, 220, 230, and 240. Additionally or alternatively, sensor electronics module 204 may also be in wireless communication (e.g., directly or indirectly) with one or more medical devices, such as medical devices 208 (individually referred to herein as medical device 208 and collectively referred to herein as medical devices 208), and/or one or more other non-analyte sensors 206 (individually referred to herein as non-analyte sensor 206 and collectively referred to herein as non-analyte sensor 206).

Additionally or alternatively, a continuous analyte sensor 202 may comprise a sensor for detecting and/or measuring analyte(s). The continuous analyte sensor 202 may be a multi-analyte sensor configured to continuously measure two or more analytes or a single analyte sensor configured to continuously measure a single analyte as a non-invasive device, a subcutaneous device, a transcutaneous device, a transdermal device, and/or an intravascular device. Additionally or alternatively, the continuous analyte sensor 202 may be configured to continuously measure analyte levels of a user using one or more measurement techniques, such as enzymatic, chemical, physical, electrochemical, spectrophotometric, polarimetric, calorimetric, iontophoretic, radiometric, immunochemical, and the like. In certain aspects the continuous analyte sensor 202 provides a data stream indicative of the concentration of one or more analytes in the user. The data stream may include raw data signals, which are then converted into a calibrated and/or filtered data stream used to provide estimated analyte value(s) to the user.

Additionally or alternatively, continuous analyte sensor 202 may be a multi-analyte sensor, configured to continuously measure multiple analytes in a user's body. For example, additionally or alternatively, the continuous multi-analyte sensor 202 may be a single multi-analyte sensor configured to measure potassium and glucose in the user's body.

Additionally or alternatively, one or more multi-analyte sensors may be used in combination with one or more single analyte sensors. As an illustrative example, a multi-analyte sensor may be configured to continuously measure potassium and glucose and may, in some cases, be used in combination with an analyte sensor configured to measure only lactate levels. Information from each of the multi-analyte sensor(s) and single analyte sensor(s) may be combined to provide decision support using methods described herein.

Additionally or alternatively, sensor electronics module 204 includes electronic circuitry associated with measuring and processing the continuous analyte sensor data, including prospective algorithms associated with processing and calibration of the sensor data. Sensor electronics module 204 can be physically connected to continuous analyte sensor(s) 202 and can be integral with (non-releasably attached to) or releasably attachable to continuous analyte sensor(s) 202. Sensor electronics module 204 may include hardware, firmware, and/or software that enables measurement of levels of analyte(s) via a continuous analyte sensor(s) 202. For example, sensor electronics module 204 can include a potentiostat, a power source for providing power to the sensor, other components useful for signal processing and data storage, and a telemetry module for transmitting data from the sensor electronics module to one or more display devices. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor.

Display devices 210, 220, 230, and/or 240 are configured for displaying displayable sensor data, including analyte data, which may be transmitted by sensor electronics module 204. Each of display devices 210, 220, 230, or 240 can include a display such as a touchscreen display 212, 222, 232, /or 242 for displaying sensor data to a user and/or receiving inputs from the user. For example, a graphical user interface (GUI) may be presented to the user for such purposes. In some embodiments, the display devices may include other types of user interfaces such as a voice user interface instead of, or in addition to, a touchscreen display for communicating sensor data to the user of the display device and/or receiving user inputs. Display devices 210, 220, 230, and 240 may be examples of display device 107 illustrated in FIG. 1 used to display sensor data to a user of FIG. 1 and/or receive input from the user.

In some embodiments, one, some, or all of the display devices are configured to display or otherwise communicate the sensor data as it is communicated from the sensor electronics module (e.g., in a data package that is transmitted to respective display devices), without any additional prospective processing required for calibration and real-time display of the sensor data.

The plurality of display devices may include a custom display device specially designed for displaying certain types of displayable sensor data associated with analyte data received from the sensor electronics module. Additionally or alternatively, the plurality of display devices may be configured for providing alerts/alarms based on the displayable sensor data. Display device 210 is an example of such a custom device. In some embodiments, one of the plurality of display devices is a smartphone, such as display device 220 which represents a mobile phone, using a commercially available operating system (OS), and configured to display a graphical representation of the continuous sensor data (e.g., including current and historic data). Other display devices can include other hand-held devices, such as display device 230 which represents a tablet, display device 240 which represents a smart watch, medical device 208 (e.g., a peritoneal dialysis machine or a hemodialysis machine), and/or a desktop or laptop computer (not shown).

Because different display devices provide different user interfaces, content of the data packages (e.g., amount, format, and/or type of data to be displayed, alarms, and the like) can be customized (e.g., programmed differently by the manufacture and/or by an end user) for each particular display device. Accordingly, additionally or alternatively, a plurality of different display devices can be in direct wireless communication with a sensor electronics module (e.g., such as an on-skin sensor electronics module 204 that is physically connected to continuous analyte sensor(s) 202) during a sensor session to enable a plurality of different types and/or levels of display and/or functionality associated with the displayable sensor data. Additionally or alternatively, the type of alarms customized for each particular display device, the number of alarms customized for each particular display device, the timing of alarms customized for each particular display device, and/or the threshold levels configured for each of the alarms (e.g., for triggering) are based on output 144 (e.g., as mentioned, output 144 may be indicative of the current health of a user, the state of a user's glucose and/or potassium, and/or current treatment recommended to a user) stored in user profile 118 for each user.

As mentioned, sensor electronics module 204 may be in communication with a medical device 208. Medical device 208 may be a passive device in some example embodiments of the disclosure. For example, medical device 208 may be a dialysis machine (e.g., peritoneal dialysis machine or hemodialysis machine) for filtering a user's blood. For a variety of reasons, it may be desirable for such a dialysis machine to receive and track potassium, glucose, phosphate, bicarbonate, calcium, magnesium, sodium, albumin, creatinine, cystatin C, and/or blood urea nitrogen transmitted from continuous analyte monitoring system 104, where continuous analyte sensor 202 is configured to measure potassium, glucose, phosphate, bicarbonate, calcium, magnesium, sodium, and/or blood urea nitrogen. In another example, medical device 208 may be an insulin pump for administering insulin to a user. For variety of reasons, it may be desirable for such an insulin pump to receive and track potassium, glucose, and insulin values from continuous analyte monitoring system 104, where continuous analyte sensor 202 is configured to measure potassium, glucose, and/or insulin.

Further, as mentioned, sensor electronics module 204 may also be in communication with other non-analyte sensors 206. Non-analyte sensors 206 may include, but are not limited to, an altimeter sensor, an accelerometer sensor, a temperature sensor, a respiration rate sensor, a sweat sensor, etc. Non-analyte sensors 206 may also include monitors such as heart rate monitors, ECG monitors, blood pressure monitors, impedance sensor, pulse oximeters, caloric intake monitors, and medicament delivery devices. One or more of these non-analyte sensors 206 may provide data to decision support engine 114 described further below. In some aspects, a user may manually provide some of the data for processing by training system 140 and/or decision support engine 114 of FIG. 1.

Additionally or alternatively, the non-analyte sensors 206 may be combined in any other configuration, such as, for example, combined with one or more continuous analyte sensors 202. As an illustrative example, a non-analyte sensor, e.g., a heart rate sensor, may be combined with a continuous analyte sensor 202 configured to measure potassium to form a potassium/heart rate sensor used to transmit sensor data to sensor electronics module 204 using common communication circuitry. As another illustrative example, a non-analyte sensor, e.g., a heart rate sensor, may be combined with a multi-analyte sensor 202 configured to measure potassium and glucose to form a potassium/glucose/heart rate sensor used to transmit sensor data to the sensor electronics module 204 using common communication circuitry.

Additionally or alternatively, a wireless access point (WAP) may be used to couple one or more of continuous analyte monitoring system 104, the plurality of display devices, medical device(s) 208, and/or non-analyte sensor(s) 206 to one another. For example, WAP 138 may provide Wi-Fi and/or cellular connectivity among these devices. Near Field Communication (NFC) and/or Bluetooth may also be used among devices depicted in diagram 200 of FIG. 2.

FIG. 3 illustrates example inputs and example metrics that are calculated based on the inputs for use by the decision support system of FIG. 1, according to some embodiments disclosed herein. In particular, FIG. 3 provides a more detailed illustration of example inputs and example metrics introduced in FIG. 1.

FIG. 3 illustrates example inputs 128 on the left, application 106 and decision support engine 114 including DAM 116 in the middle, and metrics 130 on the right. Additionally or alternatively, each one of metrics 130 may correspond to one or more values, e.g., discrete numerical values, ranges, or qualitative values (high/medium/low, stable/unstable, etc.). Application 106 obtains inputs 128 through one or more channels (e.g., manual user input, sensors/monitors, other applications executing on display device 107, EMRs, etc.). As mentioned previously, additionally or alternatively, inputs 128 may be processed by DAM 116 and/or decision support engine 114 to output metrics 130. Inputs and metrics 130 may be used by decision support engine 114 to provide decision support to the user. For example, inputs 128 and metrics 130 may be used by training system 140 to train and deploy one or more machine learning models for use by decision support engine 114 for providing the decision support outputs described above.

Additionally or alternatively, starting with inputs 128, food consumption information may include information about one or more of meals, snacks, and/or beverages, such as one or more of the size, content (milligrams (mg) of potassium, glucose, lactate, sodium, carbohydrate, fat, protein, etc.), sequence of consumption, and time of consumption. Additionally or alternatively, food consumption may be provided by a user through manual entry, by providing a photograph through an application that is configured to recognize food types and quantities, and/or by scanning a bar code or menu. In various examples, meal size may be manually entered as one or more of calories, quantity (e.g., “three cookies”), menu items (e.g., “Royale with Cheese”), and/or food exchanges (e.g., 1 fruit, 1 dairy). In some examples, meal information may be received via a convenient user interface provided by application 106.

Additionally or alternatively, food consumption information may relate to glucose consumed by the user. Glucose for consumption may include any natural or designed food or beverage that contains glucose, dextrose or carbohydrate, such as glucose tablet, a banana, or bread, for example. Additionally or alternatively, food consumption information may relate to potassium consumed by the user. Potassium for consumption may include any natural or designed food or beverage that contains potassium, such as a potassium tablet, an electrolyte drink containing potassium, or a banana, for example.

Additionally or alternatively, exercise information is also provided as an input. Exercise information may be any information surrounding activities, such as activities requiring physical exertion by the user. For example, exercise information may range from information related to low intensity (e.g., walking a few steps) and high intensity (e.g., five mile run) physical exertion. Additionally or alternatively, exercise information may be provided, for example, by an accelerometer sensor on a wearable device such as a watch, fitness tracker, and/or patch. Additionally or alternatively, exercise information may also be provided through manual user input and/or through a surrogate sensor and prediction algorithm measuring changes to heart rate (or other cardiac metrics). When predicting that a user is exercising based on his/her sensor data, the user may be asked to confirm if exercise is occurring, what type of exercise, and/or the level of strenuous exertion being used during the exercise over a specific period. This data may be used to train the system 100 to learn about the user's exercise patterns to reduce the need for confirmation questions as time progresses.

Additionally or alternatively, user statistics, such as one or more of age, height, weight, BMI, body composition (e.g., % body fat), stature, build, or other information may also be provided as an input. Additionally or alternatively, user statistics are provided through a user interface, by interfacing with an electronic source such as an electronic medical record, and/or from measurement devices. Additionally or alternatively, the measurement devices include one or more of a wireless, e.g., Bluetooth-enabled, weight scale and/or camera, which may, for example, communicate with the display device 107 to provide user data.

Additionally or alternatively, medical treatment information is also provided as an input. Medical treatment information may include information about medications, medical treatments, and/or health treatments. Medical treatment information may include the type, dosage, timing, frequency, and/or other such treatment parameters (e.g., composition (e.g., dialysate composition), concentration, flow rate, volume, and/or dialysis treatment parameters, etc.) of one or more medication and/or treatments administered to the user. As mentioned herein, the medical treatment information may include information about one or more glycemic controlling medication, one or more drugs known to damage the kidney, one or more drugs known to control the complications of kidney disease that are prescribed to the user, and/or one or more medications for treating one or more symptoms of kidney disease, hyperkalemia, hypokalemia, diabetes, and/or other conditions and diseases the user may have. Medical treatment information may include information regarding treatments for kidney disease, including dialysis (e.g., hemolysis and/or peritoneal dialysis). Medical treatment information may also include information regarding different lifestyle habits, medical treatments, surgical procedures, and/or other non-invasive procedures recommended by the user's physician. For example, the user's physician may recommend a user increase/decrease their potassium intake, exercise for a minimum of thirty minutes a day, change a medication, and/or change a treatment, to maintain, and/or improve, kidney health, glucose homeostasis, general health, etc. Additionally or alternatively, medical treatment information may be provided through manual user input.

Additionally or alternatively, analyte sensor data may also be provided as input, for example, through continuous analyte monitoring system 104. Additionally or alternatively, analyte sensor data may include glucose data (e.g., a user's glucose values) measured by at least a continuous glucose sensor (or multi-analyte sensor configured to measure at least glucose) that is a part of continuous analyte monitoring system 104. Additionally or alternatively, analyte sensor data may include potassium data measured by at least a potassium sensor (or multi-analyte sensor configured to measure at least potassium) that is a part of continuous analyte monitoring system 104. Additionally or alternatively, analyte sensor data may include lactate data measured by at least a lactate sensor (or multi-analyte sensor configured to measure at least lactate) that is a part of continuous analyte monitoring system 104. Additionally or alternatively, analyte sensor data may include insulin data measured by at least an insulin sensor (or multi-analyte sensor configured to measure at least insulin) that is a part of continuous analyte monitoring system 104. Additionally or alternatively, analyte sensor data may include pyruvate data measured by at least a pyruvate sensor (or multi-analyte sensor configured to measure at least pyruvate) that is a part of continuous analyte monitoring system 104. Additionally or alternatively, analyte sensor data may include ketone data measured by at least a ketone sensor (or multi-analyte sensor configured to measure at least ketone) that may be a part of continuous analyte monitoring system 104.

Additionally or alternatively, input may also be received from one or more non-analyte sensors, such as non-analyte sensors 206 described with respect to FIG. 2. Input from such non-analyte sensors 206 may include information related to a heart rate, heart rate variability (e.g., the variance in time between the beats of the heart), ECG data, a respiration rate, oxygen saturation, a blood pressure, or a body temperature (e.g., to detect illness, physical activity, etc.) of a user. Additionally or alternatively, electromagnetic sensors may also detect low-power radio frequency (RF) fields emitted from objects or tools touching or near the object, which may provide information about user activity or location.

Additionally or alternatively, input received from non-analyte sensors may include input relating to a user's insulin delivery. In particular, input related to the user's insulin delivery may be received, via a wireless connection on a smart insulin pen, via user input, and/or from an insulin pump. Insulin delivery information may include one or more of insulin volume, time of delivery, etc. Other parameters, such as insulin action time, insulin activity rate or duration of insulin action, may also be received as inputs.

Additionally or alternatively, input received from non-analyte sensors may include input relating to a user's dialysis treatment. In particular, input related to the user's dialysis treatment may be received, via a wireless connection on a dialysis machine, via user input, and/or from a dialysis machine. Dialysis machine information may include one or more of dialysate concentration, volume, time of delivery, flow rate, cycles, membrane type, machine type, etc.

Additionally or alternatively, time may also be provided as an input, such as time of day or time from a real-time clock. For example, additionally or alternatively, input analyte data may be timestamped to indicate a date and time when the analyte measurement was taken for the user.

User input of any of the above-mentioned inputs 128 may be through a user interface, such a user interface of display device 107 of FIG. 1. User inputs may also include user symptom data—such as tingling, nausea, vertigo, faintness, muscle weakness, or heart palpitations. Such symptom data could be a sign of electrolyte imbalance and may help correlate analyte parameters to symptoms and be used for training the algorithm/ML models.

As described above, additionally or alternatively, DAM 116 and/or decision support engine (e.g., using one or more trained models) determines or computes the user's metrics 130 based on inputs 128. An example list of metrics 130 is shown in FIG. 3.

Additionally or alternatively, analyte levels may be determined from sensor data (e.g., analyte measurements obtained from a continuous analyte sensor 202 of continuous analyte monitoring system 104). For example, analyte levels refer to time-stamped analyte levels or values that are continuously generated and stored over time. Additionally or alternatively, an analyte level may be a glucose level determined from a continuous glucose sensor. Additionally or alternatively, an analyte level may be a potassium level determined from a continuous potassium sensor. Additionally or alternatively, an analyte level may be one or more of lactate, insulin, phosphate, bicarbonate, calcium, magnesium, sodium, and/or blood urea nitrogen.

Additionally or alternatively, an analyte baseline may be determined from sensor data (e.g., analyte measurements obtained from a continuous analyte sensor 202 of continuous analyte monitoring system 104). Additionally or alternatively, an analyte baseline may be determined for one or more analytes, including potassium, glucose, lactate, insulin, phosphate, bicarbonate, calcium, and magnesium, sodium, albumin, creatinine, and/or blood urea nitrogen. An analyte baseline represents a user's normal analyte levels during periods where significant fluctuations in analyte level are typically not expected. For example, a user's potassium is generally expected to remain constant over time, unless challenged through an action such as the consumption of potassium or potassium rich foods, or changed as a result of declining kidney health or kidney function.

Further, a user may have a different baseline for a certain analyte compared to other users. For example, each user may have a different potassium baseline. Additionally or alternatively, a user's analyte baseline may be determined by calculating an average of analyte levels of the user over a specified amount of time where significant fluctuations are not expected (e.g., where no external conditions exist that would affect the analyte baseline exist). Additionally or alternatively, DAM 116 may continuously calculate an analyte baseline (e.g., a potassium baseline, a glucose baseline, etc.), time-stamp the calculated analyte baseline, and store the corresponding information in the user's profile 118.

In certain other embodiments, to calculate an analyte baseline, DAM 116 may use analyte levels measured over a period of time where the user is, at least for a subset of the period of time, engaging in an external event, condition, or activity that would affect the analyte baseline (e.g., dialysis treatment, administration of medication, exercise, consuming food, etc.). In such embodiments, DAM 116 may, in some examples, first identify which measured analyte levels are not to be used for calculating the analyte baseline by identifying which analyte levels have been affected by an event, condition, or activity, and then exclude such measurements when calculating the analyte baseline of the user. In other examples, DAM 116 may identify which measured analyte levels have been affected by an external event, condition, or activity and then calculate a baseline using only analyte levels which have been affected by the external event. The baseline may then be associated with and stored for the external event, condition, or activity. For example, a potassium-dialysis baseline may be calculated by first identifying which measured potassium levels have been affected by a dialysis treatment (e.g., potassium levels measured during the effective period of dialysis treatment), and then calculate a baseline using only those potassium levels. Effective period of dialysis treatment may include the treatment period during which the user performs dialysis using, e.g., a dialysis machine, and/or a post-treatment period, which starts from when the user stops the dialysis session but is still experiencing the effects of the dialysis.

Additionally or alternatively, whether an analyte level threshold has been reached is determined based on sensor data (e.g., analyte levels obtained from a continuous analyte sensor 202 of a continuous analyte monitoring system 104), health/sickness metrics (e.g., described in more detail below), disease stage metrics (e.g., described in more detail below) and/or medical treatment parameter (e.g., described in more detail below). Additionally or alternatively, an analyte threshold level may be determined for one or more analytes, including potassium, glucose, lactate, insulin, phosphate, bicarbonate, calcium, magnesium, sodium, creatinine, albumin, and/or blood urea nitrogen. Additionally or alternatively, a threshold level may be consistent across all users. Additionally or alternatively, a threshold level may be inputted by an end user. Additionally or alternatively, a threshold level may be an absolute maximum or an absolute minimum analyte level. Additionally or alternatively, threshold levels may change over time and/or be adjusted based on sensor data, disease stages, comorbidities, medical treatments, and/or user input. For example, a threshold level may be different during time periods in which a user is engaging in an external condition that would affect the analyte level (e.g., dialysis, exercise, consuming a meal, administering a medication).

In some instances, an external event, condition, or activity affecting the patient's analyte levels may be location of an analyte sensor in relation to a medication administration site. For example, an analyte sensor may be worn on a user's body close to a dialysis port (e.g., peritoneal dialysis port). Additionally or alternatively, the patient's analyte levels may be adjusted based on known or determined proximity to a medication administration site. For example, a glucose sensor located close to a peritoneal dialysis port may have artificially inflated glucose levels during dialysis treatment and, therefore, glucose levels during dialysis treatment may be adjusted to account for the inflated glucose levels. In another example where a glucose sensor is located close to a peritoneal dialysis port, glucose measurements during dialysis treatment may be excluded. In some embodiments, decision support engine 114 may alert a user to an external condition affecting analyte data and recommend that the user relocate an analyte sensor.

Additionally or alternatively, analyte level rates of change may be determined from sensor data (e.g., analyte measurements obtained from a continuous analyte sensor 202 of continuous analyte monitoring system 104). Additionally or alternatively, the analyte level rates of change may be one or more of potassium level rates of change, glucose level rates of change, lactate level rates of change, phosphate level rates of change, bicarbonate rates of change, calcium level rates of change, magnesium level rates of change, sodium level rates of change, and/or blood urea nitrogen level rates of change. For example, a potassium level rate of change refers to a rate that indicates how one or more time-stamped potassium levels change in relation to one or more other time-stamped potassium levels. Analyte level rates of change may be determined over one or more seconds, minutes, hours, days, etc.

Additionally or alternatively, determined analyte level rates of change may be marked as “increasing rapidly” or “decreasing rapidly”. As used herein, “rapidly” may describe analyte level rates of change that are clinically significant and pointing towards a trend of the analyte level of the patient likely breaching a threshold level within a next period of defined time.

A predictive trend (e.g., produced by decision support engine 114 using one or more trained models) may, in some cases, indicate that a patient is likely to hit, for example, an absolute maximum analyte level within a specified imminent time period based on the determined analyte level rate of change. Accordingly, such an analyte level rate of change may be marked as “increasing rapidly”. Similarly, a predictive trend (e.g., produced by decision support engine 114 using one or more trained models) may, in some cases, indicate that a patient is likely to hit a lower threshold within a specified imminent time period based on the analyte level rate of change determined. Accordingly, such an analyte level rate of change may be marked as “decreasing rapidly”.

Some medical treatments may affect accurate measurements of the rate at which analyte levels change. Therefore, during such treatments, it may be desirable to adjust which analyte trends are clinically significant and indicate a patient is likely to breach a threshold level within a defined period of time. One such treatment is dialysis. Because dialysis treatment acts a secondary analyte filter, a rate of change classified as “decreasing mildly” may actually be a rate of change that is “decreasing rapidly”. During effective periods of dialysis treatment, predictive analyte trends may be adjusted to compensate for the effects of dialysis on measured analyte rates of change. For example, a rate of change classified as “increasing mildly” may actually be a rate of change that is “rapidly increasing”. Additionally or alternatively, the adjustments may be based on the type of medical treatment. Additionally or alternatively, the adjustments may be based on different treatment parameters. Additionally or alternatively, the adjustments may be based on the analyte measured. Additionally or alternatively, the adjustments may be based on whether the analyte is increasing or decreasing. For example, during dialysis treatment, no adjustment may be needed for an increasing rate of change, but an adjustment (e.g., 50% more rapid) rate of change may be needed for a decreasing rate of change.

Additionally or alternatively, baseline analyte rates of change may be determined from baselines determined for a user over time. For example, a potassium-dialysis baseline rate of change refers to a rate that indicates how one or more time-stamped potassium-dialysis baselines for a user change in relation to one or more other time-stamped potassium-dialysis baselines for the same user. Analyte rates of change may be determined over one or more seconds, minutes, hours, days, etc. Additionally or alternatively, analyte baseline values at different time points may be determined for the user. For example, baseline before a dialysis session may be used to inform what composition of dialysate would be optimal. A baseline in between sessions, e.g., a fasting morning baseline, could be helpful to provide exercise and diet recommendations, and as that value changes, when and whether another dialysis session is needed could be determined. Another analyte baseline could be collected pre-exercise, where the type of exercise (duration, intensity, etc.) may be recommended based on the baseline. Also, a post exercise analyte (or non-analyte) baseline could be indicative of how the user's exercise performance is helping their health. For example, blood pressure post exercise could show that the exercise helped lower blood pressure.

Additionally or alternatively, an analyte clearance rate may be determined from sensor data (e.g., analyte levels obtained from a continuous analyte sensor 202 of continuous analyte monitoring system 104) following the consumption of a known, or estimated, amount of that analyte. Additionally or alternatively, an analyte clearance rate may be determined by calculating a slope between an initial high analyte level (e.g., highest analyte level during a period of increasing analyte levels) and a subsequent low analyte level (e.g., lowest analyte level during a period following increased analyte levels). Additionally or alternatively, an analyte clearance rate may be determined for one or more analytes, including potassium, glucose, lactate, insulin, phosphate, bicarbonate, calcium, and magnesium, sodium, albumin, creatinine, and/or blood urea nitrogen. Analyte clearance rates calculated over time may be time-stamped and stored in the user's profile 118.

Additionally or alternatively, analyte clearance rates analyzed over time may be indicative of changes in kidney function and/or homeostasis. For example, the slope of a curve of potassium clearance during a first time period (e.g., after consuming a known amount of potassium) compared to the slope of a curve of potassium clearance during a second time period (e.g., after consuming the same amount of potassium) may be indicative of a kidney's ability to function and more particularly, to maintain potassium homeostasis (e.g., potassium clearance rate may be slower when a user's kidney is impaired than when a user's kidney is healthy). The slope of a curve of analyte data over many different periods of time (e.g., over 5 minutes, 10 minutes, 35 minutes, one hour, one day, a week, or a month, for example) may be compared to determine a trend of the slope of a curve of analyte data, which may be indicative of a user's kidney function over time. In another example, a change between the slope of a curve of glucose clearance during a first time period and a second time period may be indicative of a kidney's ability to function and maintain glucose homeostasis.

Additionally or alternatively, analyte clearance rates may be determined during periods of time when a user is engaging in an external event, condition, or activity that may affect the analyte clearance rate. For example, an analyte clearance rate may be determined during the treatment period of a medical treatment (e.g., dialysis, diuretic, insulin, etc.). An analyte clearance rate may also be monitored prior to or after the treatment period. Additionally or alternatively, analyte clearance rates associated with an external event, condition, or activity may be used to indicate the effect the external condition had on the analyte levels (e.g., the effect of a medical treatment, the effect of exercise, the effect of consuming a meal).

Additionally or alternatively, analyte trends may be determined based on analyte levels measured over certain periods of time (e.g., potassium levels over time, glucose levels over time, lactate levels over time, insulin levels over time, phosphate levels over time, bicarbonate levels over time, calcium levels over time, magnesium levels over time, sodium levels over time, blood nitrogen urea levels over time, etc.). Additionally or alternatively, analyte trends may be determined based on analyte baselines over time. Additionally or alternatively, analyte trends may be determined based on analyte levels over time. Additionally or alternatively, analyte trends may be determined based on analyte rates of change over time. Additionally or alternatively, analyte trends may be determined based on analyte clearance rates over time.

Additionally or alternatively, insulin sensitivity may be determined using historical data, real-time data, or a combination thereof, and may, for example, be based upon one or more inputs 128, such as one or more of food consumption information, continuous analyte sensor data, non-analyte sensor data (e.g., insulin delivery information from an insulin device), etc. Insulin sensitivity refers to how responsive a user's cells are to insulin. Improving insulin sensitivity for a user may help to reduce insulin resistance in the user.

Additionally or alternatively, insulin on board may be determined using analyte sensor data (e.g., insulin measurements obtained from an insulin sensor of a continuous analyte monitoring system 104), non-analyte sensor data input (e.g., insulin delivery information) and/or known or learned (e.g., from user data) insulin time action profiles, which may account for both basal metabolic rate (e.g., uptake of insulin to maintain operation of the body) and insulin usage driven by activity or food consumption.

Additionally or alternatively, health and sickness metrics may be determined, for example, based on one or more of user input (e.g., pregnancy information or known sickness or disease information), from physiologic sensors (e.g., temperature), activity sensors, or a combination thereof. Additionally or alternatively, based on the values of the health and sickness metrics, for example, a user's state may be defined as being one or more of healthy, ill, rested, or exhausted.

Additionally or alternatively, disease stage metrics, such as for kidney disease, may be determined, for example, based on one or more of user input or output provided by decision support engine 114 illustrated in FIG. 1. Additionally or alternatively, example disease stages for kidney disease, may include AKI, stage 1 CKD with normal or high GFR (e.g., GFR>90 mL/min), stage 2 mild CKD (e.g., GFR=60-89 mL/min), stage 3A moderate CKD (e.g., GFR=45-59 mL/min), stage 3B moderate CKD (e.g., GFR=30-44 mL/min), stage 4 severe CKD (e.g., GFR=15-29 mL/min), and stage 5 end stage CKD (e.g., GFR<15 mL/min). Additionally or alternatively, example disease stages may be represented as a GFR value/range, severity score, and the like.

Additionally or alternatively, the meal state metric may indicate the state the user is in with respect to food consumption. For example, the meal state may indicate whether the user is in one of a fasting state, pre-meal state, eating state, post-meal response state, or stable state. Additionally or alternatively, the meal state may also indicate nourishment on board, e.g., meals, snacks, or beverages consumed, and may be determined, for example from food consumption information, time of meal information, and/or digestive rate information, which may be correlated to food type, quantity, and/or sequence (e.g., which food/beverage was eaten first).

Additionally or alternatively, meal habits metrics are based on the content and the timing of a user's meals. For example, if a meal habit metric is on a scale of 0 to 1, the better/healthier meals the user eats the higher the meal habit metric of the user will be to 1, in an example. Also, the more the user's food consumption adheres to a certain time schedule or a recommended diet, the closer their meal habit metric will be to 1, in the example.

Additionally or alternatively, medical treatment adherence is measured by one or more metrics that are indicative of how committed the user is towards their medical treatment regimen. Additionally or alternatively, medical treatment adherence metrics are calculated based on one or more of the timing of when the medical treatment is administered (e.g., whether the administration is on time or on schedule), the type of medical treatment (e.g., whether the administration is the right type of medical treatment), and the treatment parameters of the medical treatment (e.g., whether the administration is at the right treatment parameters). Additionally or alternatively, medical treatment adherence of a user may be determined by monitoring the medical treatment administration and timing as well as parameters of such medical treatment administration. Monitoring the medical treatment administration may involve receiving information about the treatment administration through user input, from a medical device 208, and/or from analyte monitoring system 104.

Additionally or alternatively, the activity level metric may indicate the user's level of activity. Additionally or alternatively, the activity level metric be determined, for example based on input from an activity sensor or other physiologic sensors, such as non-analyte sensors 206. Additionally or alternatively, the activity level metric may be calculated by DAM 116 based on one or more of inputs 128, such as one or more of exercise information, non-analyte sensor data (e.g., accelerometer data), time, user input, etc. Additionally or alternatively, the activity level may be expressed as a step rate of the user. Activity level metrics may be time-stamped so that they can be correlated with the user's analyte levels at the same time.

Additionally or alternatively, exercise regimen metrics may indicate one or more of what type of activities the user engages in, the corresponding intensity of such activities, frequency the user engages in such activities, etc. Additionally or alternatively, exercise regimen metrics may be calculated based on one or more of non-analyte sensor data input (e.g., non-analyte sensor data input from an accelerometer, a heart rate monitor, a respiration rate sensor, etc.), calendar input, user input, etc.

Additionally or alternatively, body temperature metrics may be calculated by DAM 116 based on inputs 128, and more specifically, non-analyte sensor data from a temperature sensor. Additionally or alternatively, heart rate metrics (e.g., including heart rate and heart rate variability) may be calculated by DAM 116 based on inputs 128, and more specifically, non-analyte sensor data from a heart rate sensor. Additionally or alternatively, respiratory metrics may be calculated by DAM 113 based on inputs 128, and more specifically, non-analyte sensor data from a respiratory rate sensor.

### Example Methods and Systems for Providing Decision Support Around Diabetes and Kidney Disease

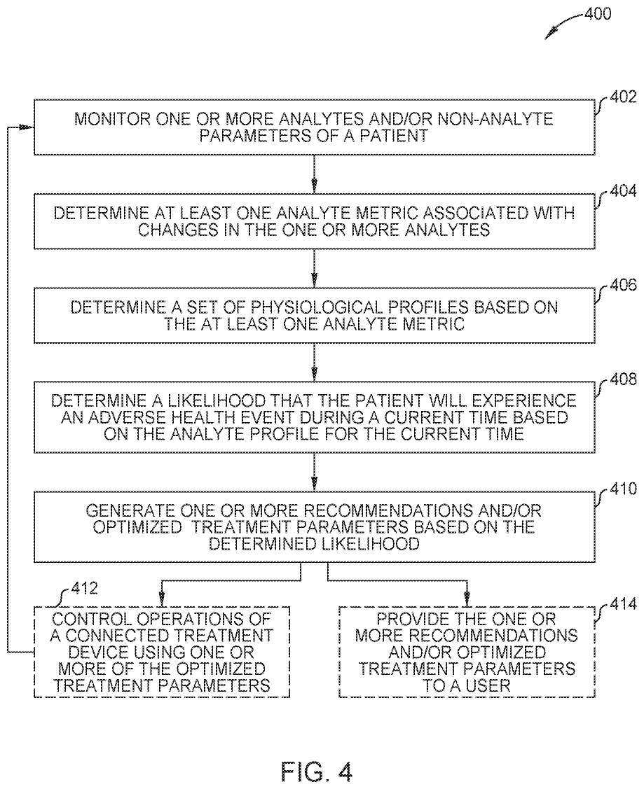

FIG. 4 is a flow diagram illustrating an example method 400 for providing decision support using a continuous analyte monitoring system including, at least, a continuous analyte sensor capable of monitoring at least one of glucose or potassium, in accordance with certain example aspects of the present disclosure. For example, method 400 may be performed to provide decision support to a user, using a continuous analyte monitoring system 104 including, at least, a continuous analyte sensor 202, as illustrated in FIGS. 1 and 2.

Method 400 may be performed by decision support system 100 to collect and/or generate data such as inputs 128 and metrics 130, including for example, analyte data, patient information, and non-analyte sensor data during various time periods (e.g., a treatment period, pre-treatment period, and/or post-treatment period), to create various corresponding physiological profiles that can be used to (1) identify risk of adverse events during the various time periods based on the corresponding physiological profiles, (2) make patient-specific treatment decisions or recommendations to help address the identified risk of adverse events, including providing recommended treatment parameters for administration of medical treatments and/or automatically controlling the operations of one or more medical devices (e.g., dialysis machine, insulin pump, etc.) based on such recommended treatment parameters.

For example, decision support system 100 may perform method 400 by monitoring one or more analytes of a patient during a pre-treatment, treatment, or post-treatment period of a medical treatment. The decision support system 100 may then determine one or more analyte metrics (e.g., analyte rate of change, analyte clearance rate, etc.) associated with the monitored analytes prior to, during, and after a treatment period of a medical treatment and generate a pre-treatment, treatment, and post-treatment physiological profiles, respectively. These physiological profiles may be indicative of the patient's analyte metrics, such as analyte rate of change and/or analyte clearance rate, during the corresponding time periods. As such, once these profiles are created the decision support system 100 may then determine a likelihood that the patient will experience an adverse health event during pre-treatment, treatment, and post-treatment time periods.

For example, using a treatment profile created for the user by monitoring the user's analyte metrics during one or more previous treatment periods, decision support system 100 is able to predict the likelihood of an adverse event during a current treatment period based on the user's current analyte and/or non-analyte information, current treatment parameters, and other information to determine a likelihood of an adverse event taking place during the treatment period. Based on the determined likelihood, decision support system 100 may then generate optimized treatment parameters and/or recommendations. For example, if the determined likelihood is above a certain threshold, decision support system 100 may generate optimized treatment parameters and/or recommendations to reduce the likelihood of the adverse event.

As another example, using a post-treatment profile created for the user by monitoring the user's analyte metrics during one or more previous post-treatment periods, decision support system 100 is able to predict the likelihood of an adverse event during a current post-treatment period based on the user's current analyte and/or non-analyte information, treatment parameters used during the treatment session, and other information to determine a likelihood of an adverse event taking place during the post-treatment period. Based on the determined likelihood, decision support system 100 may then generate optimized post-treatment recommendations. For example, if the determined likelihood is above a certain threshold, decision support system 100 may generate optimized post-treatment recommendations to reduce the likelihood of the adverse event.

As another example, using a pre-treatment profile and/or treatment profile created for the user by monitoring the user's analyte metrics during one or more previous pre-treatment periods and/or during treatment periods, decision support system 100 is able to predict the likelihood of an adverse event during a current post-treatment period based on the user's current analyte and/or non-analyte information, treatment parameters used during the treatment session, and other information to determine a likelihood of an adverse event taking place during the post-treatment period. Based on the determined likelihood, decision support system 100 may then generate optimized post-treatment recommendations. For example, if the determined likelihood is above a certain threshold, decision support system 100 may generate optimized post-treatment recommendations to reduce the likelihood of the adverse event.

As yet another example, using a pre-treatment profile created for the user by monitoring the user's analyte metrics during one or more previous pre-treatment periods, decision support system 100 is able to predict the likelihood of an adverse event during a current pre-treatment period based on the user's current analyte and/or non-analyte information and other information to determine a likelihood of an adverse event taking place during the pre-treatment period. Based on the determined likelihood, decision support system 100 may then generate optimized pre-treatment recommendations. For example, if the determined likelihood is above a certain threshold, decision support system 100 may generate optimized pre-treatment recommendations to reduce the likelihood of the adverse event.

In certain embodiments, a pre-treatment profile created for the user may allow decision support system 100 to predict the likelihood of an adverse event during treatment or post-treatment based on the user's pre-treatment analyte data. For example, a dialysis session with a higher flow rate may cause a high rate of change for insulin and/or glucose levels, which may not result in adverse events during dialysis, but may increase the likelihood of an adverse event (e.g. hypoglycemia) after the dialysis treatment.

Additionally or alternatively, decision support system 100 presented herein may be configured to predict the effect of medical treatment on kidney function and provide decision support for management of medical treatments affecting kidney function and, thereby, the user's analyte clearance rates. In particular, a patient may experience periods of time where the patient's kidney function is different or reacts differently than the patient's typical kidney function due to a medical treatment affecting kidney function. Examples include worsening glycemic status or hypertension causing a decrease in kidney function, or acute kidney injury resulting in an abrupt decline in kidney function. Thus, by predicting the effects of a medical treatment on the patient's kidney function based on sensor data (e.g., generated by a continuous analyte sensor 202), decision support system 100 presented herein may generate optimal treatment parameters to reduce the likelihood of adverse health effects associated with a medical treatment affecting kidney function, which may be critical to improving patient care and reducing deterioration of kidney function.

Additionally or alternatively, decision support engine 114 of decision support system 100 may use various algorithms or artificial intelligence (AI) models, such as machine-learning models, trained based on patient-specific data and/or population data to predict analyte metrics (e.g., rate of change and/or clearance rates) associated with pre-treatment, treatment, and/or post-treatment time periods and decision support for the management of the medical treatment. The algorithms and/or machine-learning models may take into account one or more inputs 128 and/or metrics 130 described with respect to FIG. 3 for a patient when predicting analyte metrics (e.g., rate of change and/or clearance rates) and generating decision support for at least one of the pre-treatment, treatment, and/or post-treatment time periods.

Additionally or alternatively, the one or more machine learning models may include input, single output (MISO) models that are trained to each predict metrics associated with pre-treatment, treatment, and/or post-treatment time periods for a different analyte. For example, one model may be trained to predict glucose clearance rates, while another model may be trained to predict potassium clearance rates.

Additionally or alternatively, the one or more machine learning models may include input, multiple output (MIMO) models that is trained to predict metrics associated with different periods for multiple analytes. For example, one model may be trained to predict both glucose clearance rates and potassium clearance rates. Additionally or alternatively, the model may be trained to output a vector having multiple values, where each value corresponds to a different predicted analyte clearance rate that the model is trained to generate.

Additionally or alternatively, the one or more machine learning models may include MISO models that are trained to generate an optimized medical treatment parameter for a medical treatment (e.g., type, dosage, timing, frequency, composition, concentration, flow rate, volume, etc.). For example, one model may be trained to generate an optimized type of a medical treatment, while another model may be trained to generate an optimized frequency of a medical treatment.

Additionally or alternatively, the one or more machine learning models may include MIMO models that are trained to generate optimized medical treatment parameters for a medical treatment (e.g., type, dosage, timing, frequency, composition, concentration, flow rate, volume, etc.). For example, a single model may be trained to generate an optimized type and frequency of a medical treatment. Additionally or alternatively, the model may be trained to output a vector having multiple values, where each value corresponds to a different treatment parameter for which the model is trained to generate the optimized treatment parameter.

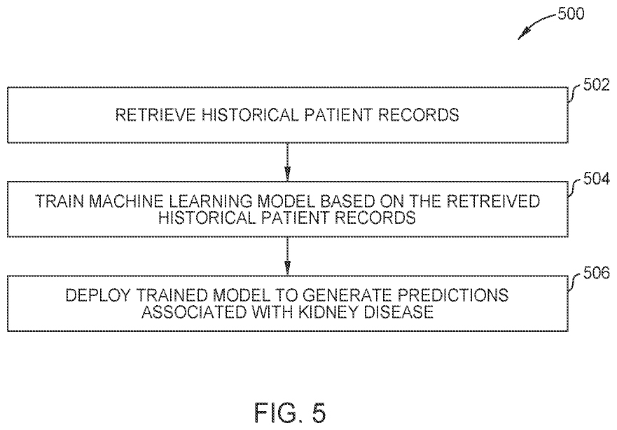

The one or more machine-learning models described herein for making such predictions may be initially trained using population data. A method for training the one or more machine learning models may be described in more detail below with respect to FIG. 5.

Additionally or alternatively, as an alternative to using machine learning models, decision support engine 114 may use rule-based models to determine the effect of a medical treatment on the patient's physiology (e.g., analyte metrics, such as clearance rates) and provide decision support for the management of medical treatments. Rule-based models involve using a set of rules for analyzing data. These rules are sometimes referred to as ‘If statements’ as they tend to follow the line of ‘If X happens then do or conclude Y’. In particular, decision support engine 114 may apply rule-statements (e.g., if, then statements) to determine how the patient's physiology may be impacted by a medical treatment and provide decision support for conducting the medical treatment based on the determination.

Such rules may be defined and maintained by decision support engine 114 in a reference library. For example, the reference library may maintain ranges of analyte clearance rates which may be mapped to different likelihoods of adverse events. In another example, the reference library may maintain ranges of treatment parameters which may be mapped to different effects on analyte clearance rates. Additionally or alternatively, such rules may be determined based on empirical research or an analysis of historical patient records, such as the records stored in historical records database 112. In some cases, the reference library may become very granular. For example, other factors may be used in the reference library to create such “rules”. Other factors may include gender, age, diet, disease history, family disease history, body mass index (BMI), etc. Increased granularity may provide more accurate outputs.

At block 402, method 400 begins by continuously monitoring one or more analytes of a patient, such as user 102 illustrated in FIG. 1. The one or more analytes monitored may include at least one of glucose and potassium. Block 402 may be performed by continuous analyte monitoring system 104 illustrated in FIGS. 1 and 2, and more specifically, continuous analyte sensor(s) 202 illustrated in FIG. 2, additionally or alternatively. For example, continuous analyte monitoring system 104 may comprise a continuous analyte sensor 202 configured to measure the patient's analyte levels prior to, during, and/or after a treatment period of a medical treatment. Examples of medical treatments include exercise, diet, medication intake, or dialysis. The effective period of a medical treatment may be one or more periods of time during which a medical treatment induces a biological response in a user. A biological response may include activity, absorption, pharmacodynamics, affinity, and/or efficacy of a medical treatment on a patient.

While the main analytes for measurement described herein are glucose and/or potassium, additionally or alternatively, other analytes may be considered. In particular, combining analyte data from two or more analytes may help to further inform the effect of a medical treatment on the patient's physiology and providing decision support for the management of treatments for patients with kidney disease. For example, monitoring additional types of analytes such as glucose, potassium, lactate, insulin, phosphate, bicarbonate, calcium, magnesium, sodium, albumin, creatinine, and/or BUN measured by continuous analyte monitoring system 104, may provide additional insight into the effect of a medical treatment on the patient's physiology and providing decision support for such a medical treatment.

Additionally or alternatively, the additional insight gained from using a combination of analytes may increase the accuracy of the predicted effect of a medical treatment on patient physiology, such as adverse events. For example, the probability of accurately predicting the effect of a medical treatment on patient physiology may be a function of the number of analytes measured for a patient. In some examples, a probability of accurately predicting the effect of a medical treatment on patient physiology using only potassium data (in addition to other non-analyte data) may be less than a probability of accurately predicting the effect of a medical treatment on patient physiology using potassium and glucose data (in addition to other non-analyte data), which may also be less than a probability of accurately predicting the effect of a medical treatment on patient physiology using potassium, glucose, and sodium data (in addition to other non-analyte data) for analysis.

Additionally or alternatively described herein, analyte combinations, e.g., measured and collected by one (e.g., multi-analyte) or more sensors, for predicting the effect of a medical treatment on patient physiology and providing decision support for the management and adjustment of medical treatments, include at least two of glucose, potassium, lactate, insulin, phosphate, bicarbonate, calcium, magnesium, sodium, and BUN; however, other analyte combinations may be considered. Because the kidney processes and metabolizes many different analytes, additional analyte data may improve the accuracy of a prediction on the effect of a medical treatment on patient physiology. In one example, the prediction may include a prediction of the likelihood of an adverse event occurring during or post treatment.

For example, lactate levels may be associated with glucose, insulin, and potassium metabolism. Lactate levels may also be used to detect consumption of food, exercise, rest, infection, and/or stress. Therefore, the additional insights gained from lactate levels, which may be indicative of metabolism and kidney function, as well as different bodily states (e.g., food consumed, exercise, rest, infection, stress, etc.), may result in generating more complete physiological profiles, which are then used to determine a likelihood of adverse events during pre-treatment, treatment, and post-treatment periods. For example, measurements of glucose levels and lactate levels, especially when monitored in combination with medication information, may be used to determine if the patient is at risk of developing lactic acidosis.

In another example, calcium levels may be associated with calcium metabolism. Calcium homeostasis is maintained by the kidney and calcium levels may be used to indicate kidney function. Therefore, the additional insights gained from calcium levels may result in generating more complete physiological profiles, which are then used to determine a likelihood of adverse events during pre-treatment, treatment, and post-treatment periods. Additionally, calcium homeostasis may be affected by certain medical treatments (e.g., dialysis) and calcium levels may be used to generate optimal treatment parameters for such medical treatments.

In another example, increased levels of phosphates in the blood (e.g., hyperphosphatemia) may be associated with CKD. Hyperphosphatemia (e.g., abnormally high serum phosphate levels) can result from increased phosphate intake, decreased phosphate excretion, or a disorder that shifts intracellular phosphate to extracellular space. This increase in serum phosphate levels is associated with decreased renal ion excretion, as well as, the use of medications to reduce the progression of CKD or to control associated diseases such as diabetes mellitus and heart failure. Furthermore, phosphate homeostasis is maintained by the kidney and phosphate levels may be used to indicate kidney function. Therefore, the additional insights gained from phosphate levels may improve may result in generating more complete physiological profiles, which are then used to determine a likelihood of adverse events during pre-treatment, treatment, and post-treatment periods. Additionally, phosphate homeostasis may be affected by certain medical treatments (e.g., dialysis) and phosphate levels may be used to generate optimal treatment parameters for such medical treatments.

In yet another example, bicarbonate homeostasis is maintained by the kidney and bicarbonate levels may be used to indicate kidney function. Therefore, the additional insights gained from bicarbonate levels may result in generating more complete physiological profiles, which are then used to determine a likelihood of adverse events during pre-treatment, treatment, and post-treatment periods. Additionally, bicarbonate homeostasis may be affected by certain medical treatments (e.g., dialysis) and bicarbonate levels may be used to generate optimal treatment parameters for such medical treatments.

In yet another example, magnesium homeostasis is maintained by the kidney and magnesium levels may be used to indicate kidney function. Therefore, the additional insights gained from magnesium levels may result in generating more complete physiological profiles, which are then used to determine a likelihood of adverse events during pre-treatment, treatment, and post-treatment periods. Additionally, magnesium homeostasis may be affected by certain medical treatments (e.g., dialysis) and magnesium levels may be used to generate optimal treatment parameters for such medical treatments.

In yet another example, sodium homeostasis is maintained by the kidney and sodium levels may be used to indicate kidney function. Therefore, the additional insights gained from sodium levels may result in generating more complete physiological profiles, which are then used to determine a likelihood of adverse events during pre-treatment, treatment, and post-treatment periods. Additionally, sodium homeostasis may be affected by certain medical treatments (e.g., dialysis) and sodium levels may be used to generate optimal treatment parameters for such medical treatments.

In another example, the liver produces ammonia, which contains nitrogen, after the liver breaks down proteins used by cells in the body. The nitrogen combines with other elements, such as carbon, hydrogen and oxygen, to form urea, which is a chemical waste product. The urea travels from the liver to the kidneys through the bloodstream. Healthy kidneys filter urea and remove other waste products from the blood, and the filtered waste products leave the body through urine. Accordingly, BUN levels (e.g., the levels of nitrogen content in urea) may provide insight into kidney health and function. Thus, a patient experiencing high levels of measured extracellular potassium is assumed to have damaged kidney function, and may also be expected to be experiencing high levels of measured BUN (e.g., given a damaged kidney would not likely be capable of filtering urea and removing other waste products from the blood). Accordingly, BUN levels may be used to indicate kidney function. Therefore, the additional insights gained from BUN levels may result in generating more complete physiological profiles, which are then used to determine a likelihood of adverse events during pre-treatment, treatment, and post-treatment periods. Additionally, BUN metabolism may be affected by certain medical treatments (e.g., dialysis) and BUN levels may be used to generate optimal treatment parameters for such medical treatments.

In another example, creatinine is produced primarily as a byproduct of muscle and protein metabolism. It is cleared by the kidneys and is therefore a useful metric of kidney health. Therefore, additional insight gained from creatinine levels may result in generating more complete physiological profiles, which are then used to determine a likelihood of adverse events during pre-treatment, treatment, and post-treatment periods.

In another example, cystatin C is a protein that is often used as a marker of kidney health, even in early stages of kidney disease. As kidney health begins to decline, the amount of cystatin C in the body begins to rise, even in the early stages of kidney disease. Therefore, additional insight gained from cystatin C levels may result in generating more complete physiological profiles, which are then used to determine a likelihood of adverse events during pre-treatment, treatment, and post-treatment periods.

In another example, serum albumin is a protein produced by the liver and which keeps homeostasis, particularly the extracellular fluid volume, or oncotic pressure. Albumin levels can change significantly during dialysis, and thus impact blood pressure and fluid volume. It is therefore important to measure albumin during dialysis.

Additionally, other analytes may be used to indicate other effects (e.g., not kidney function effects) of a medical treatment affecting a patient's kidney function. For example, lactate levels measured in close proximity to a peritoneal dialysis port may indicate sepsis of a peritoneal dialysis port.

In addition, to continuously monitoring one or more analytes of a patient during a plurality of time periods to obtain analyte data at block 402, optionally, additionally or alternatively, method 400 may also include monitoring other sensor data (e.g., non-analyte data) during the plurality of time periods using one or more other non-analyte sensors or devices (e.g., such as non-analyte sensors 206 and/or medical device 208 of FIG. 2).

As mentioned previously, non-analyte sensors and devices may include one or more of, but are not limited to, an insulin pump, an acoustic sensor, a haptic sensor, an ECG sensor or heart rate monitor, a blood pressure sensor, a respiratory sensor, a peritoneal dialysis machine, a hemodialysis machine, sensors or devices provided by display device 107 (e.g., accelerometer, camera, global positioning system (GPS), heart rate monitor, etc.) or other user accessories (e.g., a smart watch), or any other sensors or devices that provide relevant information about the user. Metrics, such as metrics 130 illustrated in FIG. 3, may be calculated using measured data from one or more of these additional sensors. As illustrated in FIG. 3, metrics 130 calculated from non-analyte sensor or device data may include heart rate (including heart rate variability), respiratory rate, etc. Additionally or alternatively, described in more detail below, metrics 130 calculated from non-analyte sensor or device data may be used to create physiological profiles and, therefore, to further inform the analysis around medical treatments affecting the patient's physiology.

Additionally or alternatively, one or more of these non-analyte sensors and/or devices may be worn by a user to aid in the detection of periods of increased physical exertion by the user. Such non-analyte sensors and/or devices may include an accelerometer, an ECG sensor, a blood pressure sensor, blood oxygen/oximetry sensor, atmospheric pressure sensor, atmospheric oxygen sensor, a heart rate monitor, an impedance sensor, an insulin pump, a dialysis machine (e.g., a peritoneal dialysis machine, a hemodialysis machine, etc.), sensors or devices provided by display device 107 (e.g., accelerometer, camera, global positioning system (GPS), heart rate monitor, etc.) or other user accessories (e.g., a smart watch), or any other sensors or devices that provide relevant information about the user. Analyte metrics, such as analyte clearance rates and analyte levels may be affected by exercise. Because of these effects, additionally or alternatively, analyte data collected during exercise may be excluded from information used for determining a likelihood of an adverse health event. However, additionally or alternatively, analyte data may be correlated with exercise and determination of a likelihood of an adverse health event may be based on analyte data collected during exercise. For example, a determined likelihood of an adverse health event during exercise may be based on analyte data collected during exercise. In some embodiments, collecting atmospheric sensor readings enables a decision support algorithm to take location-related features associated with a location of a user into account when determining treatment recommendations and/or treatment parameters for the user.

Additionally or alternatively, one or more of these non-analyte sensors and/or devices may be worn by a user to aid in the prediction of an adverse event during various time periods. Additionally or alternatively, one or more non-analyte sensors and/or devices that may be worn by a patient may include a blood pressure sensor. Blood pressure measurements collected from a blood pressure sensor may be used to provide additional insight around the likelihood of adverse events. In particular, CKD and high blood pressure are closely related. Typically, as blood pressure rises, kidney function declines. Accordingly, the assessment of blood pressure levels of a patient during various time periods may provide additional insight into the kidney health of the patient, which may for example translate into lower potassium clearance rates. Thus, a patient experiencing high levels of measured extracellular potassium and is assumed to have damaged kidney function (e.g., given excess potassium is not being filtered from the body), may also be expected to be experiencing high blood pressure levels.

Additionally or alternatively, one or more non-analyte sensors and/or devices that may be worn or used by a patient may include an ECG sensor and/or a heart rate monitor. As is known in the art, an ECG device is a device that measures the electric activity of the heartbeat. Any morphological changes or interval changes in ECG signals may be used in combination with analyte data to provide a more accurate determination of risk of an adverse health event. Additionally or alternatively, heart rate measurements, as well as heart rate variability information, collected from an ECG sensor and/or a heart rate monitor may be used in combination with analyte data to more accurately determine the risk of an adverse health event, including hyperkalemia and/or experiencing one or more cardiac event(s) (e.g., arrhythmia and/or sudden cardiac death).

Additionally or alternatively, one or more analyte and/or non-analyte sensors and/or devices may be used or worn by a user to aid in determining when each of the pre-treatment, treatment, and/or post-treatment periods start and end. For example, non-analyte sensors and/or devices may be used to determine whether a medical treatment, such as dialysis, is in progress as well as the initiation and/or termination of a medical treatment. Such non-analyte sensors and/or devices may include an accelerometer, an ECG sensor, a blood pressure sensor, a heart rate monitor, an impedance sensor, an insulin pump, a dialysis machine, and the like. In some embodiments, non-analyte sensors and/or devices may only be worn or used when a medical treatment is administered or for administrating a medical treatment. For example, a dialysis machine may only be used by a user for administering a dialysis treatment. Therefore, additionally or alternatively, a user wearing or using a non-analyte sensor and/or device may indicate a medical treatment is currently being administered and user is currently in a treatment period. Similarly, additionally or alternatively, a user not wearing or using a non-analyte sensor and/or device may indicate a medical treatment is not currently being administered and a user is not currently in an effective period for the medical treatment. Additionally or alternatively, non-analyte sensors and/or devices may be worn or used at times when a medical treatment is not being administered. For example, an insulin pump may be continuously worn by a user, even when insulin is not administered at all times. Therefore, in such embodiments, a user wearing or using a non-analyte sensor and/or device may not necessarily indicate that a medical treatment is being administered and/or if a user is currently in an effective period for the medical treatment.

Additionally or alternatively, one or more non-analyte sensors and/or devices worn or used by a user may indicate treatment parameters and other medical treatment information relating to a medical treatment. Such non-analyte sensors and/or devices may include an accelerometer, an ECG sensor, a blood pressure sensor, a heart rate monitor, an impedance sensor, an insulin pump, a dialysis machine, and the like. Additionally or alternatively, detected treatment parameters may form part of the medical treatment information provided as part of inputs 128. As mentioned herein, medical treatment information may include the type, dosage, timing, frequency, and/or other like treatment parameters (e.g., composition, concentration, flow rate, volume, etc.) of one or more medication and/or treatments. Additionally or alternatively, a medical treatment may be administered to a user through a non-analyte sensor and/or device as well as provide non-analyte data. For example, a dialysis machine may be used to administer dialysis treatment to a user and may be use to provide non-analyte data. Additionally or alternatively, treatment parameters are provided by a non-analyte sensor and/or device. Additionally or alternatively, treatment parameters provided by a non-analyte sensor and/or device may be confirmed by a user.

At block 404, method 400 continues by processing the analyte data to determine at least one analyte rate of change associated with changes in the one or more analytes. Additionally or alternatively, block 404 may be performed prior to, during, and/or after the treatment period of a medical treatment. For example, in some embodiments, one or more analyte metrics associated with a pre-treatment period for a medical treatment are determined and used to determine the pre-treatment profile for the patient. As another example, in some embodiments, one or more analyte metrics associated with the effective period of a medical treatment are determined and used to determine a treatment profile for the patient. As a further example, in some embodiments, one or more analyte metrics associated with the post-treatment period of a medical treatment are determined and used to determine a post-treatment profile for the patient. Block 404, additionally or alternatively, may be performed by decision support engine 114.

As mentioned, an analyte rate of change refers to a rate that indicates the change of one or more time-stamped analyte levels in relation to one or more other time-stamped analyte levels. Additionally or alternatively, analyte rates of change are used to determine physiological profiles (e.g., a pre-treatment profile, a treatment profile, and/or a post-treatment profile for a patient). In some embodiments, at least one analyte rate of change for the patient may be calculated and used for creating physiological profiles. For example, potassium levels and/or rate(s) of change prior to, during, and after a medical treatment may be used as input to create pre-treatment, treatment, and post-treatment physiological profiles. In another example, glucose levels and/or rate(s) of change prior to, during, and after a medical treatment may be used as input to create pre-treatment, treatment, and post-treatment physiological profiles

In some embodiments, analyte clearance rates prior to, during, and after a medical treatment may be used as input to create pre-treatment, treatment, and post-treatment physiological profiles. For example, clearance rates of potassium, glucose, lactate and/or other analytes described herein may be used as input to create physiological profiles.

At block 406, method 400 continues by determining a set of physiological profiles based on the at least one analyte metric that was determined in block 404 over various corresponding time periods. Block 406 may be performed by decision support engine 114 illustrated in FIG. 1. Additionally or alternatively, block 406 may be performed to create and update a pre-treatment profile, treatment profile, and a post-treatment profile for the patient based on various triggers. For example, as described above, decision support engine 114 may use analyte data, non-analyte data, and/or other types of data to determine when a treatment period starts and ends, when a post-treatment period starts and ends, and when a pre-treatment period starts and ends. As an example, decision support engine 114 may continuously perform blocks 402 and 404 for a new patient over the first few weeks of the patients using decision support system 100. During these first few weeks, decision support engine 114 creates and updates a pre-treatment profile, treatment profile, and a post-treatment profile for the patient with data corresponding to each of these time periods.

A physiological profile may describe one or more analyte metric patterns (e.g., analyte rate of change pattern, clearance rate pattern, etc.) of a patient during a corresponding time period. For example, the patient's treatment profile may indicate a historical pattern of analyte metrics of the patient during treatment periods. The patient's post-treatment profile may indicate a historical pattern of analyte metrics of the patient during post-treatment periods. A post-treatment period starts after the treatment period but is associated with a higher likelihood of adverse events resulting from the treatment. The patient's pre-treatment profile may indicate historical pattern of analyte metrics of the patient during time periods that fall outside of the treatment and the post-treatment periods.

Examples of analyte metrics include analyte clearance rates, such as glucose clearance rates and potassium clearance rates. As previously discussed, a user's analyte clearance rate may be different from the user's baseline analyte clearance rates during various time periods. In some cases, medical treatments (e.g., dialysis) may affect a patient's analyte clearance rates. For example, in some embodiments, the patient may have different glucose clearance rates before, during, and after a dialysis treatment. As another example, in some embodiments, the potassium clearance rate of a patient may have different glucose clearance rates before, during, and after a dialysis treatment.

In some embodiments, for each patient, decision support engine 114 creates and updates three physiological profiles: a pre-treatment profile, a treatment profile, and a post-treatment profile. A treatment profile describes one or more patterns of analyte metrics of the patient during the treatment period of the medical treatment. A post-treatment profile describes one or more patterns of analyte metrics of the patient during a post-treatment period that follows the treatment period. The post-treatment period may be associated with a heightened likelihood of adverse events resulting from the treatment. In some embodiments, the post-treatment period is a period having a predefined length after the treatment period, such as a period of one or more minutes, hours, days, or weeks after the effective period.

In some embodiments, one or more physiological measures of the patient (e.g., a heart rate of the patient, a temperature of the patient, etc.) after the treatment period of the treatment are measured to determine whether the physiological measures satisfy the requirements of the post-treatment period. If the physiological measures of the patient after the treatment period satisfy requirements of the post-treatment period, the patient is determined to be in the post-treatment period. However, as soon as the physiological measures of the patient change such that those measures no longer satisfy requirements of the post-treatment period, then the patient is determined to be outside of the post-treatment period and in a pre-treatment period. A pre-treatment profile describes one or more patterns of analyte metrics of the patient during the pre-treatment period that is the period outside of the treatment period and the post-treatment period.

The profiles created for the patient when the patient first starts using decision support system 100 are then continuously updated by the decision support engine 114. For example, consider a patient that starts using decision support system 100 on day 1 but does not receive dialysis treatment on day 1, receives dialysis treatment on day 2, does not receive dialysis treatment on day 3, receives dialysis treatment on days 4, and does not receive dialysis treatment on days 5-6. In this example, if the post-treatment period has a predefined length of one day, then the decision support engine 114 may first generate the pre-treatment profile for the patient based on analyte metrics associated with day 1. On day 2, data associated with a time period prior to the treatment period is used to update the pre-treatment profile. Once the treatment starts on day 2, the decision support engine 114 may generate the treatment profile for the patient based on analyte metrics associated with the treatment period during day 2. Next, the decision support engine 114 may generate the post-treatment profile for the patient based on analyte rates of change associated with the time period after treatment has ended on day 2 and part of day 3.

As further described below, using the profiles created and updated over day 1, day 2, and day 3, the decision support engine 114 may predict the likelihood of adverse events and/or provide decision support outputs (e.g., optimal treatment parameters, recommendations, etc.) during corresponding time periods on day 4, day 5, and day 6. For example, using the patient's treatment profile, decision support engine 114 may predict the likelihood of adverse events and/or provide decision support outputs during the treatment period on day 4. Subsequently, the decision support engine 114 may use the post-treatment profile to predict the likelihood of adverse events and/or provide decision support outputs during the post-treatment periods associated with day 4 and day 5. Afterward, the decision support engine 114 may use the pre-treatment profile to predict the likelihood of adverse events and/or provide decision support outputs for day 6. During each of these days, decision support engine 114 also continues to update the patient's profile using data collected over the corresponding time periods.

Additionally or alternatively, at block 402, continuous analyte monitoring system 104 may continuously monitor glucose and potassium levels of a patient during pre-treatment, treatment, and post-treatment periods. Additionally or alternatively, the potassium data and the glucose data for each period may then be used to determine glucose and potassium metrics, such as glucose and potassium clearance rates during similar periods in the future and provide decision support for the management of the medical treatment. For example, the potassium data and glucose data may indicate (1) analyte clearance rates of the patient before, during, and/or after the effective period of the medical treatment as well as (2) a likelihood of an adverse health event occurring before, during, and/or after the effective period of the medical treatment.

In one particular example, glucose and/or potassium measurements generated during a treatment period of a medical treatment may be used to determine glucose and/or potassium clearance rates for the treatment period. Then, the determined glucose and/or potassium clearance rates for the treatment period are used to generate or update a treatment profile that is reflective of the patient's pattern of glucose and/or potassium clearance rates over one or more treatment periods associated with one or more treatment sessions. The treatment profile as generated or updated can then be used to determine a likelihood that the patient will experience an adverse event during the effective period of a future medical treatment (e.g., a future medical treatment having the same treatment type).

As another example, glucose and/or potassium measurements generated during a post-treatment period, that follows a treatment period of a medical treatment, may be used to determine analyte glucose and/or potassium rates for the post-treatment period. Then, the determined glucose and/or potassium rates for the post-treatment period are used to generate or update a post-treatment profile that is reflective of the patient's pattern of glucose and/or potassium clearance rate over one or more post-treatment periods. The post-treatment profile as generated or updated can then be used to determine a likelihood that the patient will experience an adverse event during the post-treatment period of a future medical treatment (e.g., a future medical treatment having the same treatment type).

As yet another example, if a glucose and/or potassium measurement is within a pre-treatment period, then the glucose and/or potassium measurement may be used to determine analyte clearance rates for the pre-treatment period. Then, the determined glucose and/or potassium clearance rates for the pre-treatment period are used to generate or update a pre-treatment profile that that is reflective of the patient's pattern of glucose and/or potassium clearance rate over one or more pre-treatment periods. The pre-treatment profile as generated or updated can then be used to determine a likelihood that the patient will experience an adverse event in the pre-treatment period of a future medical treatment (e.g., a future medical treatment having the same treatment type). Additionally, pre-treatment glucose and/or potassium measurements may be used to customize a treatment session for a user in order to prevent an adverse event.

Method 400 continues at block 408 by decision support engine 114 determining a likelihood that the patient will experience an adverse health event during a current time (i.e., the time at which the decision support engine 114 determines the likelihood) based on the physiological profiles generated at block 406. In particular, decision support engine 114 may: (i) classify the current time based on its relationship with the treatment period of a treatment (i.e., classify the current time as being one of the treatment period, a pre-treatment period, or a post-treatment period), (ii) determine the physiological profile for the current time based on the classification of the current time, (iii) retrieve the pattern of analyte metrics described by the physiological profile for the current time, and (iv) determine the likelihood of an adverse health event based on the retrieved pattern of analyte metrics.

A pattern of analyte metrics indicate metrics that can be expected to be experienced by a user during a corresponding period and, therefore, can be used to predict the likelihood of a user experiencing an adverse event. In such an example, the pattern of analyte metrics may include an average analyte rate of change, an average analyte clearance rate, analyte rates of changes at different times during a corresponding period, analyte clearance rates at different times during a corresponding period, a standard deviation of analyte levels during a specified period, an average standard deviation of analyte levels over subsequent periods of time, etc. An adverse health event, additionally or alternatively, may be one or more of: hypokalemia, hyperkalemia, hypoglycemia, hyperglycemia, cardiac event(s), mortality, and the like. In one example, the likelihood of a hyperkalemia event may increase if the expected potassium clearance rate for the current time is below a threshold potassium clearance rate required to maintain a target level of patient health.

For example, in some embodiments, the decision support engine 114 determines whether the current time is in a pre-treatment period, a treatment period, or a post-treatment period. If the decision support engine 114 determines that the current time is in the treatment period, the decision support engine 114 determines the adverse event likelihood for the current time based on the pattern of analyte metrics described by the treatment profile generated and/or updated at block 406. Furthermore, if the decision support engine 114 determines that the current time is in the post-treatment period, the decision support engine 114 determines the adverse event likelihood for the current time based on the pattern of analyte metrics described by the post-treatment profile of the user. Moreover, if the decision support engine 114 determines that the current time is in the pre-treatment period, the decision support engine 114 determines the adverse event likelihood for the pattern of analyte metrics described by the pre-treatment profile

As mentioned, different methods for determining the likelihood that the patient will experience an adverse health event may be used by decision support engine 114. Additionally or alternatively, rule-based model(s) may be used. A rule-based model may include rules may take into account the current period the user is in, user's current analyte levels, treatment parameters, physiological profiles, and/or other factors. Treatment parameters may indicate the type, dosage amount, activity rate, activity duration, and/or timing of medical treatment. The user's current analyte levels may indicate the user's current potassium and/or glucose analyte levels, as well as other analytes including lactate, insulin, phosphate, bicarbonate, calcium, magnesium, sodium, and/or BUN. For example, using a rule based model, decision support engine 114 may determine that, if the user is in a treatment period, the user's current potassium and/or glucose levels are X and/or Y, the user's treatment profile indicates a Z clearance rate, and the user's treatment parameters for the treatment sessions are W, then the user's likelihood of experiencing an adverse event is Q. In another example, using a rule based model, decision support engine 114 may determine that, if the user is in a post-treatment period, the user's current potassium and/or glucose levels are X and/or Y, the user's post-treatment profile indicates a Z clearance rate, and the user's treatment parameters for the treatment sessions were W, then the user's likelihood of experiencing an adverse event is Q. In yet another example, using a rule based model, decision support engine 114 may determine that, if the user is in a pre-treatment period, the user's current potassium and/or glucose levels are X and/or Y, the user's pre-treatment profile indicates a Z clearance rate, then the user's likelihood of experiencing an adverse event is Q. A rule-based model may be more granular and include many other rules associated with the user's demographic information and other relevant parameters.

Additionally or alternatively, machine-learning model(s) may be used to predict the likelihood of a user experiencing an adverse event during a certain period. Inputs 128 and/or metrics 130 described with respect to FIG. 3, including the user's current analyte levels, treatment parameters, expected pattern of analyte metrics (as indicated by a corresponding physiological profile) generated at block 406, and/or other relevant data points (e.g., demographic information) may be used by a machine-learning model to predict the likelihood of a user experiencing an adverse event during a certain period. For example, a model may be trained using a training dataset of historical patient records, each (1) indicating a historical patient's analyte levels time-stamped analyte levels, treatment parameters, pattern of analyte metrics (time-stamped analyte clearance rates), and/or other relevant data points (e.g., demographic information) and (2) labeled with a likelihood of an adverse event (100% being indicative of the adverse even actually having taken place).

Method 400 continues at block 410 by decision support engine 114 by generating one or more recommendations and/or optimized treatment parameters based on the likelihood determined at block 408. Additionally or alternatively, decision support engine 114 may use one or more machine-learning models, trained based on patient-specific data and/or population data, to generate optimized treatment parameters to manage medical treatments. The machine-learning models may take into account one or more inputs 128 and/or metrics 130 described with respect to FIG. 3 for a patient to determine optimal recommendations for optimized treatment parameters to reduce the determined likelihood of adverse health events during a certain period. Additionally or alternatively, as an alternative to using machine-learning models, decision support engine 114 may use one or more decision trees to provide optimized treatment parameters. The decision tree may be rule-based and provide optimized treatment parameters to reduce the determined likelihood of adverse health events during a period (e.g., treatment, pre-treatment, or post treatment period).

Optimized treatment parameters may be generated by decision support engine 114 and in some embodiments, may be recommended to a user or automatically used in controlling the operations of a medical devices, such as a treatment administration device (e.g., dialysis machine), as described further in relation to block 412. Optimized treatment parameters to manage medical treatments may, in some cases, be based on the patient's physiological profile, current analyte levels, determined likelihood of an adverse health event, current treatment parameters, and/or other relevant information. For example, optimized treatment parameters may be recommended based on the likelihood of an adverse health event determined at block 408. Additionally or alternatively, recommended treatment parameters may include type, dosage amount, activity rate, activity duration, timing, concentration, composition, flow rate, volume, and/or other treatment parameters which may be associated with a medical treatment.

Additionally or alternatively, a medical treatment may be dialysis and decision support engine 114 may generate optimized treatment parameters for dialysis. Optimized treatment parameters may include type of dialysate including composition and/or concentration, type of dialysis membrane, flow rate, timing of treatment, frequency of treatment, length of treatment, and/or any other parameter of dialysis treatment which may be adjusted to reduce the likelihood of an adverse health event during a treatment session. Adjustments of dialysis parameters during treatment may allow for reduction of adverse health events.

Flow rate is the rate at which dialysate flows through the dialysis machine and filters a patient's blood. Increasing the flow rate may increase the rate at which analytes are filtered out of a patient's blood. Flow rate may be optimized to increase or reduce filtration of analytes out of a patient's blood to reduce likelihood of an adverse health event. For example, the flow rate may be optimized by increasing flow rate to increase filtration of an analyte (e.g., potassium) in order to reduce risk of an adverse health event (e.g., hyperkalemia). In another example, the flow rate may be optimized by increasing flow rate (e.g., increased from 200 mL/min to 300 mL/min or more, for example) to reduce the risk of hypoglycemia during or after a dialysis treatment for a patient with low glucose levels and/or a history of hypoglycemia. Alternatively, the flow rate may be optimized by decreasing the flow rate (e.g., decreased from 300-500 mL/min to 200 mL/min, for example) to reduce the risk of hyperglycemia during or after a dialysis treatment for a patient with high glucose levels and/or a history of hyperglycemia.

Additionally, hemodialysis may cause release of potassium from sheer stress on red blood cells breaking from high flow rate. An optimized flow rate may be a reduced flow rate to reduce sheer stress and release of potassium during dialysis. For example, an optimized flow rate may be a reduced flow rate to prevent sheering of red blood cells. Flow rate may also be optimized to reduce the so called “rebound” effect where certain electrolytes, such as potassium, may increase following a dialysis session.

Dialysate is the mixture which flows through a hemodialysis machine or circulated through a catheter in a patient's peritoneal cavity in peritoneal dialysis. Dialysis absorbs analytes out of a patient's blood thereby reducing serum analyte concentrations. Dialysate composition and concentration may affect the amount and rate of analyte movement between the dialysate and the patient's blood. Dialysate composition and/or concentration may be optimized to reduce the risk of too much or too little movement of analytes. For example, an optimized dialysate concentration may be a reduction in dextrose concentration to reduce a patient's glucose levels and prevent hyperglycemia.

Hemodialysis treatment is often administered according to a weekly schedule with sessions occurring multiple (e.g., 2-3) times weekly lasting a prescribed length (e.g., 1 hour). A treatment schedule is prescribed to a patient and is often not adjusted based on a patient's specific characteristics. Optimizing treatment parameters may involve adjusting the dialysis treatment schedule including timing, length and frequency. For example, a patient's availability for a dialysis treatment may change (e.g., due to work, a family emergency, a financial situation, transportation, etc.), which may cause the dialysis treatment schedule to be altered. In certain embodiments, a patient may be notified, based on the patient's specific characteristics, when they are at risk of potential adverse events if they do not reschedule and/or complete a dialysis treatment in a specific time period (e.g., 3 days, for example).

Additionally or alternatively, elevated analyte levels (current or projected) and/or decrease analyte clearance rates (as indicated by a user's corresponding physiological profile) may indicate a need for a longer dialysis session and, therefore, an optimized treatment parameter may be to lengthen the dialysis treatment (e.g., by 50%). Additionally or alternatively, reduced analyte levels (current or projected) and an increased kidney function (current or projected) may indicate a desire for a shorter dialysis session and, therefore, an optimized treatment parameter may be to shorten dialysis treatment (e.g., by 25%). Additionally or alternatively, reduced glucose levels at the start of a dialysis treatment may indicate that a higher flow rate and/or a particular type of dialysate is/are desirable and, therefore, an optimized treatment parameter may be to increase flow rate (e.g., to 300-500 mL/min) for the dialysis treatment and/or use a dialysate that increases glucose levels. If a patient is at high risk for hypoglycemia during or after a dialysis treatment (e.g., based on current glucose levels, medical history of hypoglycemia, or data from prior dialysis treatment sessions demonstrating a risk of hypoglycemia), an optimized dialysis treatment parameter may be to slightly increase flow rate (e.g., from 200 mL/min to 300 mL/min or more). Additionally or alternatively, increased glucose levels at the start of a dialysis treatment may indicate that a slower flow rate would be desirable and, therefore, an optimized treatment parameter may be to decrease a flow rate (e.g., to 200 mL/min) for the dialysis treatment.

Additionally or alternatively, a patient's insulin sensitivity and/or insulin on board concentration may assist in determining optimized dialysis treatment parameters. For example, monitoring a patient's insulin concentration pre-treatment, during treatment, and/or post-treatment may inform insulin dosing recommendations, especially following a dialysis treatment session. Additionally, insulin sensitivity may be determined during dialysis by monitoring the glucose and insulin concentrations during dialysis, and comparing them to the glucose and insulin concentrations before dialysis. Based on the glucose and insulin concentrations during dialysis, decision support engine 114 may predict insulin sensitivity post-treatment to inform insulin dosing recommendations.

Currently, peritoneal dialysis treatment is often administered at night while a patient sleeps. Further, currently, similar to hemodialysis, for a peritoneal dialysis treatment, a treatment schedule is often not adjusted based on a patient's specific situation (e.g., kidney function, risk of adverse health event, etc.). However, the embodiments described herein allow for optimizing these treatment parameters based on the inputs described above. For example, additionally or alternatively, a user's current analyte levels, a user's corresponding physiological profile, a determine likelihood of adverse event, etc., may indicate that a shorter, or delayed peritoneal dialysis treatment should be considered. Additionally or alternatively, the length of treatment may be based on the user's analyte level reaching or crossing a desired analyte level instead of a determined length of time. For example, an optimal treatment parameter may be to continue dialysis treatment until the analyte level has reached a desired level (e.g., potassium at 3 moll/L) and then cease dialysis treatment. Additionally or alternatively, the length of treatment may be based on a rate of change or a predictive trend (e.g., adjusted predictive trend) of the analyte level reaching or crossing a desired analyte rate of change and then cease dialysis treatment.

Additionally or alternatively, a medical treatment may involve administration of a diuretic and decision support engine 114 may generate optimized treatment parameters for administration of a diuretic. In such cases, treatment parameters may include type, dosage, frequency, timing, and/or like treatment parameters which may be optimized to reduce the likelihood of an adverse health even during the effective period of the diuretic treatment.

Additionally or alternatively, the type of diuretic recommended may be either potassium sparing or non-potassium sparing. Additionally or alternatively, the type of diuretic recommended may be based on the determined likelihood of an adverse health event, such as hypokalemia and/or hyperkalemia. Non-potassium sparing diuretics will reduce potassium levels. Where a patient is at increased risk of hyperkalemia, for example, a non-potassium sparing diuretic may be recommended to reduce risk of hyperkalemia. However, where a patient is at increased risk of hypokalemia, for example, a potassium sparing diuretic may be recommend to avoid increasing the risk of hypokalemia. However, if a patient has developed end stage kidney failure and is receiving dialysis treatment, a diuretic may not be useful to the patient, as it requires the kidneys to be effective.

Additionally or alternatively, the frequency and/or timing of a diuretic may be recommended based on the determined likelihood of an adverse health event. Diuretics are often used to reduce blood pressure. Additionally or alternatively, treatment parameters may be optimized based on conflicting adverse health event risks. For example, non-analyte data may indicate a need to administer a diuretic to reduce risk of high blood pressure, however, analyte data may indicate a need to avoid administration of a diuretic to reduce likelihood of an adverse health event based on analyte data. In such cases, the recommended treatment parameters may be, additionally or alternatively, to delay dosage of a diuretic until analyte levels can be raised, as well as a recommendation to raise analyte levels (e.g., through consumption).

Additionally or alternatively, the dosage of a diuretic may be optimized and recommended based on a determined likelihood of an adverse health event. Optimal treatment parameters may be to adjust a dosage to reduce the likelihood of an adverse health event, including reducing a dosage, increasing a dosage, and/or adjusting dosage and other treatment parameter (e.g., frequency, type, timing, etc.). For example, an optimal treatment parameter may be a reduced dosage of a diuretic to reduce the likelihood of hypokalemia. In another example, an optimal treatment parameter may be a reduced dosage of a diuretic as well as consumption of potassium to reduce the likelihood of hypokalemia. Additionally or alternatively, one or more optimized treatment parameters may be recommended together. Additionally or alternatively, optimized treatment parameters may be dependent upon the available adjustments (e.g., only certain parameters may be changed), the type of treatment (e.g., hemodialysis, peritoneal dialysis, diuretic, etc.), user input, and other optimized parameters. For example, optimized treatment parameters for hemodialysis may be different than optimized treatment parameters for peritoneal dialysis. In another example, only frequency and timing of administration of a diuretic may be optimized treatment parameters for a diuretic but the dosage of a diuretic may be constant or set up user input (e.g., by a HCP).

Additionally or alternatively, recommendations may also include decision support recommendations to help the user prevent and/or reduce the likelihood of adverse health events during treatment and/or post-treatment periods of a medical treatment, including food intake recommendations, exercise recommendations and other medical treatment recommendations. For example, additionally or alternatively, an optimized treatment parameter may be an increased potassium level and a recommendation may be provided to a user to consume potassium (e.g., potassium supplement) to increase the user's potassium level and reduce the likelihood of hypokalemia during the treatment and/or post-treatment periods.

Additionally or alternatively, optimized treatment parameters may be accompanied by a recommendation to consult with a HCP regarding the optimized treatment parameters. For example, an optimized treatment parameter may be to discontinue use of a medical treatment. In certain cases, decision support engine 114 may also recommend to a patient to consult with a HCP before discontinuing use of a medical treatment. Additionally or alternatively, optimized treatment parameters may be provided to a HCP.

Additionally or alternatively, optimized treatment parameters may be generated for a subsequent treatment period. Subsequent treatment parameters, additionally or alternatively, may be based on optimized treatment parameters of a prior treatment period. For example, optimized treatment parameters for an effective treatment period which reduced the likelihood of an adverse health event may be recommended as initial treatment parameters for a subsequent effective treatment period.

Method 400 may continue by controlling operations of a connected treatment device using one or more of the optimized treatment parameters, at block 412, and/or providing the one or more recommendations and/or optimized treatment parameters to a user, at block 414. As discussed above, a medical device 208 may be part of continuous analyte monitoring system 104. Examples of the medical device 208 may include a dialysis machine, an insulin pump, or other treatment devices. As discussed above, in the case of a dialysis machine, the optimized treatment parameters may include optimized type, dosage amount, activity rate, activity duration, timing, concentration, composition, flow rate, volume, and/or other treatment parameters which may be associated with a dialysis machine. The operations of the dialysis machine may be controlled by the decision support engine 114, either directly or through the patient's display device (e.g., display device 107), transmitting the optimized treatment parameters to the dialysis machine and causing the dialysis machine to operate according to the treatment parameters. For example, an optimized treatment parameter may be an optimized flow rate determined at block 410. Once the optimized treatment parameter is received by the dialysis machine, the dialysis machine adjusts its “current” flow rate (i.e., flow rate with which treatment is being administered to the patient) to reach the optimized flow rate.

Additionally or alternatively, one or more of recommendations and/or optimized treatment parameters may be provided to the user though application 106. For example, any of the recommendations and/or treatment parameters described in relation to block 410 may be provided to the user though a user interface of application 106, thereby, allowing the user to manually alter the operations of a medical device (e.g., dialysis machine), manually adjust administration of a certain treatment, and/or follow the recommendations (e.g., food intake recommendations, etc.).

Further, method 400 may continually flow through block 402 through block 410 during various periods to continually control the operations of a connected treatment device using one or more of the optimized treatment parameters at block 410 and/or provide recommendations including one or more of the generated optimized treatment parameters. Treatment parameters may be continually optimized to reduce the likelihood of an adverse health event during the effective period of a medical treatment and continually monitored analyte data.

As discussed herein, machine learning models deployed by decision support engine 114 include one or more models trained by training system 140, as illustrated in FIG. 1, to provide various types of predictions, as discussed in relation to FIG. 4. FIG. 5 describes in further detail techniques for training one or more machine learning models for predicting (1) risk of adverse events during the various time periods based on the corresponding physiological profiles, (2) patient-specific treatment decisions or recommendations to help address the identified risk of adverse events. Note that a different model may be trained for each of the above predictions or outputs.

Method 500 begins, at block 502, by a training system, such as training system 140 illustrated in FIG. 1, retrieving data from a historical records database, such as historical records database 112 illustrated in FIG. 1. As mentioned herein, historical records database 112 may provide a repository of up-to-date information and historical information for (1) users of a continuous analyte monitoring system and connected mobile health application, such as users of continuous analyte monitoring system 104 and application 106 illustrated in FIG. 1, and/or (2) one or more users who are not, or were not previously, users of continuous analyte monitoring system 104 and/or application 106.

Retrieval of data from historical records database 112 by training system 140, at block 502, may include the retrieval of all, or any subset of, information maintained by historical records database 112. For example, where historical records database 112 stores information for 100,000 patients (e.g., non-users and users of continuous analyte monitoring system 104 and application 106), data retrieved by training system 140 to train one or more machine learning models may include information for all 100,000 patients or only a subset of the data for those patients, e.g., data associated with only 50,000 patients or only data from the last ten years.

As an example, integrating with on premises or cloud based medical record databases through Fast Healthcare Interoperability Resources (FHIR), web application programming interfaces (APIs), Health Level 7 (HL7), and/or other computer interface language may enable aggregation of healthcare historical records for baseline assessment in addition to the aggregation of de-identifiable patient data from a cloud based repository.

As an illustrative example, at block 502, training system 140 may retrieve information for 100,000 patients with or without varying kidney function and with or without various medical treatments known to change a patient's analyte metrics (e.g., analyte clearance rates or rate of change) in historical records database 112 to train one or more models to predict the effect of a medical treatment on a patient's analyte levels, predict likelihood of an adverse health event, and/or generate recommendations and/or optimal treatment parameters for said treatment. Each of the 100,000 patients may have a corresponding data record (e.g., based on their corresponding user profile)), stored in historical records database 112. Each user profile 118 may include information, such as information discussed with respect to FIG. 3.

The training server system 140 then uses information in each of the records to train an artificial intelligence or ML model (for simplicity referred to as “ML model” herein). Examples of types of information included in a patient's user profile were provided above. The information in each of these records may be featurized (e.g., manually or by training server system 140), resulting in features that can be used as input features for training the ML model. For example, a patient record may include or be used to generate features related to an age of a patient, a gender of the patient, an occupation of the patient, analyte levels for the patient over time, analyte level rates of change and/or trends for the patient over time, physiological parameters associated with different adverse events for the patient over time, and/or any information provided by inputs 128 and/or metrics 130, etc. Features used to train the machine learning model(s) may vary in different embodiments.

In certain embodiments, each historical patient record retrieved from historical records database 112 is further associated with a label indicating whether the patient was healthy or experienced some variation of kidney disease, whether the patient experienced an adverse event during a period of time, treatment(s), and/or similar metrics. What the record is labeled with would depend on what the model is being trained to predict.

At block 504, method 500 continues by training system 140 training one or more machine learning models based on the features and labels associated with the historical patient records. In some embodiments, the training server does so by providing the features as input into a model. This model may be a new model initialized with random weights and parameters, or may be partially or fully pre-trained (e.g., based on prior training rounds). Based on the input features, the model-in-training generates some output. Additionally or alternatively, the output may (1) indicate a risk of adverse events during the various time periods based on the corresponding physiological profiles or (2) patient-specific treatment decisions or recommendations to help address the identified risk of adverse events.

Additionally or alternatively, training system 140 compares this generated output with the actual label associated with the corresponding historical patient record to compute a loss based on the difference between the actual result and the generated result. This loss is then used to refine one or more internal weights and parameters of the model (e.g., via backpropagation) such that the model learns to predict the risk of adverse events during various time periods (or its recommended treatments) more accurately.

One of a variety of machine learning algorithms may be used for training the model(s) described above. For example, one of a supervised learning algorithm, a neural network algorithm, a deep neural network algorithm, a deep learning algorithm, etc. may be used.

At block 506, training system 140 deploys the trained model(s) to make predictions associated with kidney disease during runtime. In some embodiments, this includes transmitting some indication of the trained model(s) (e.g., a weights vector) that can be used to instantiate the model(s) on another device. For example, training system 140 may transmit the weights of the trained model(s) to decision support engine 114. The model(s) can then be used to predict (1) risk of adverse events during the various time periods based on the corresponding physiological profiles and/or (2) patient-specific treatment decisions or recommendations to help address the identified risk of adverse events.

Further, similar methods for training illustrated in FIG. 5 using historical patient records may also be used to train models using patient-specific records to create more personalized models for making predictions associated with (1) risk of adverse events during the various time periods based on the corresponding physiological profiles and/or (2) patient-specific treatment decisions or recommendations to help address the identified risk of adverse events. For example, a model trained using historical patient records that is deployed for a particular user, may be further re-trained after deployment. For example, the model may be re-trained after the model is deployed for a specific patient to create a more personalized model for the patient. The more personalized model are able to more accurately make predictions associated with (1) risk of adverse events during the various time periods based on the corresponding physiological profiles and/or (2) patient-specific treatment decisions or recommendations to help address the identified risk of adverse events.

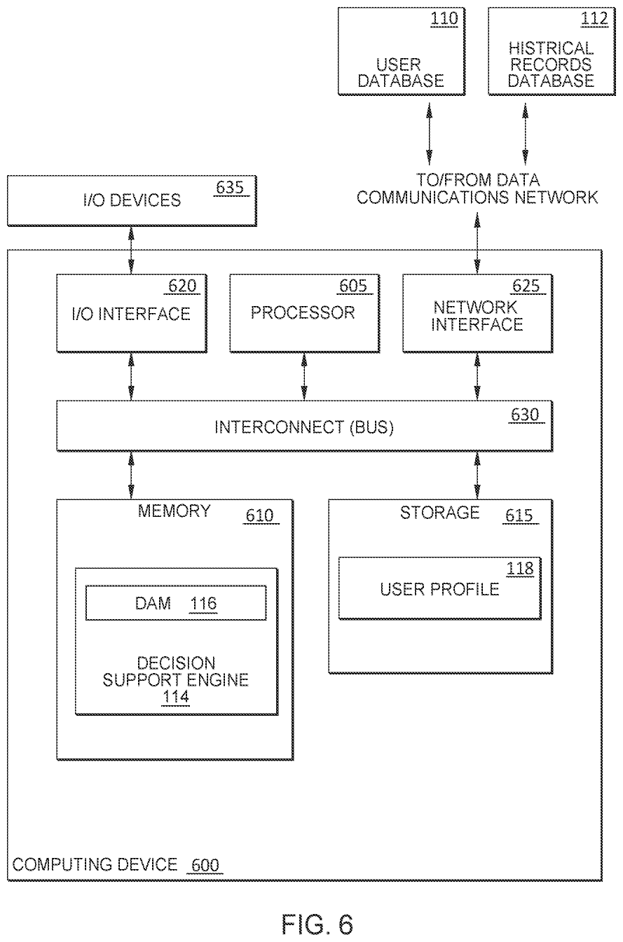

FIG. 6 is a block diagram depicting a computing device 600 configured for (1) predicting the effect of medical treatment on kidney function and (2) providing decision support (e.g., predicting optimal treatment, providing recommendations, etc.) for the management of treatments affecting kidney function. Although depicted as a single physical device, in embodiments, computing device 600 may be implemented using virtual device(s), and/or across a number of devices, such as in a cloud environment. As illustrated, computing device 600 includes a processor 605, memory 610, storage 615, a network interface 625, and one or more I/O interfaces 620. In the illustrated embodiment, processor 605 retrieves and executes programming instructions stored in memory 610, as well as stores and retrieves application data residing in storage 615. Processor 605 is generally representative of a single CPU and/or GPU, multiple CPUs and/or GPUs, a single CPU and/or GPU having multiple processing cores, and the like. Memory 610 is generally included to be representative of a random access memory (RAM). Storage 615 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, input and output (I/O) devices 635 (such as keyboards, monitors, etc.) can be connected via the I/O interface(s) 620. Further, via network interface 625, computing device 600 can be communicatively coupled with one or more other devices and components, such as user database 110 and/or historical records database 112. Additionally or alternatively, computing device 600 is communicatively coupled with other devices via a network, which may include the Internet, local network(s), and the like. The network may include wired connections, wireless connections, or a combination of wired and wireless connections. As illustrated, processor 605, memory 610, storage 615, network interface(s) 625, and I/O interface(s) 620 are communicatively coupled by one or more interconnects 630. Additionally or alternatively, computing device 600 is representative of display device 107 associated with the user. Additionally or alternatively, as discussed above, display device 107 can include the user's laptop, computer, smartphone, and the like. In another embodiment, computing device 600 is a server executing in a cloud environment.

In the illustrated embodiment, storage 615 includes user profile 118. Memory 610 includes decision support engine 114, which itself includes DAM 116. Decision support engine 114 is executed by computing device 600 to perform operations in method 400 of FIG. 4, and/or operations of method 500 in FIG. 5

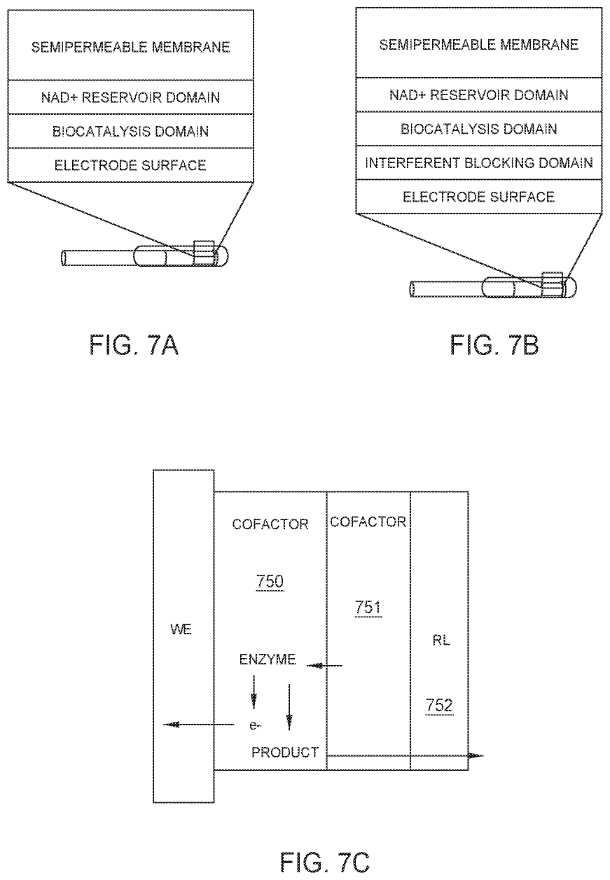

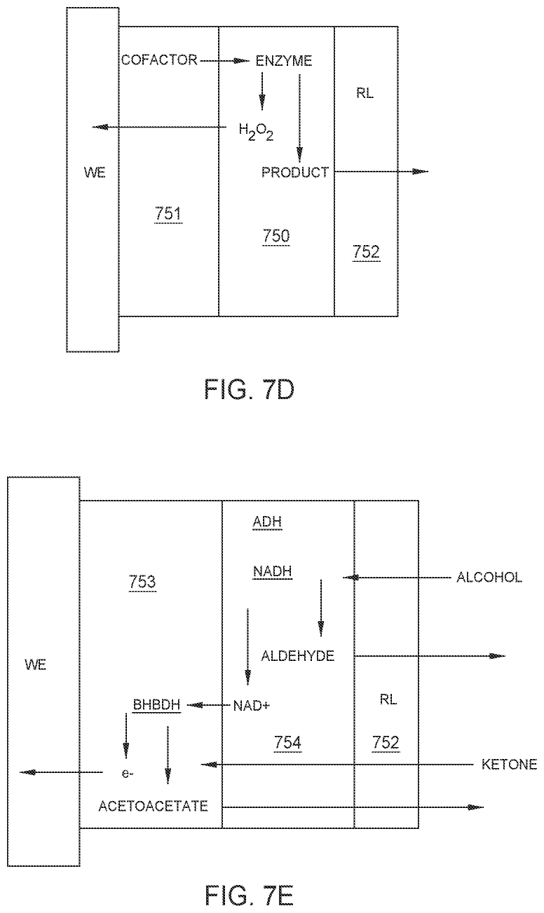

As described above, continuous analyte monitoring system 104, described in relation to FIG. 1, may be a multi-analyte sensor system including a multi-analyte sensor. FIGS. 7-11 describe example multi-analyte sensors used to measure multiple analytes.

The phrases “analyte-measuring device,” “analyte-monitoring device,” “analyte-sensing device,” and/or “multi-analyte sensor device” as used herein are broad phrases, and are to be given their ordinary and customary meaning to a person of ordinary skill in the art (and are not to be limited to a special or customized meaning), and refer without limitation to an apparatus and/or system responsible for the detection of, or transduction of a signal associated with, a particular analyte or combination of analytes. For example, these phrases may refer without limitation to an instrument responsible for detection of a particular analyte or combination of analytes. In one example, the instrument includes a sensor coupled to circuitry disposed within a housing, and configure to process signals associated with analyte concentrations into information. In one example, such apparatuses and/or systems are capable of providing specific quantitative, semi-quantitative, qualitative, and/or semi qualitative analytical information using a biological recognition element combined with a transducing (detecting) element.

The terms “biosensor” and/or “sensor” as used herein are broad terms and are to be given their ordinary and customary meaning to a person of ordinary skill in the art (and are not to be limited to a special or customized meaning), and refer without limitation to a part of an analyte measuring device, analyte-monitoring device, analyte sensing device, and/or multi-analyte sensor device responsible for the detection of, or transduction of a signal associated with, a particular analyte or combination of analytes. In one example, the biosensor or sensor generally comprises a body, a working electrode, a reference electrode, and/or a counter electrode coupled to body and forming surfaces configured to provide signals during electrochemically reactions. One or more membranes can be affixed to the body and cover electrochemically reactive surfaces. In one example, such biosensors and/or sensors are capable of providing specific quantitative, semi-quantitative, qualitative, semi qualitative analytical signals using a biological recognition element combined with a transducing (detecting) element.

The phrases “sensing portion,” “sensing membrane,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In one example, the sensing portion, sensing membrane, and/or sensing mechanism generally comprise an electrode configured to provide signals during electrochemically reactions with one or more membranes covering electrochemically reactive surface. In one example, such sensing portions, sensing membranes, and/or sensing mechanisms can provide specific quantitative, semi-quantitative, qualitative, semi qualitative analytical signals using a biological recognition element combined with a transducing (detecting) element.

The phrases “biointerface membrane” and “biointerface layer” as used interchangeably herein are broad phrases, and are to be given their ordinary and customary meaning to a person of ordinary skill in the art (and are not to be limited to a special or customized meaning), and refer without limitation to a permeable membrane (which can include multiple domains) or layer that functions as a bioprotective interface between host tissue and an implantable device. The terms “biointerface” and “bioprotective” are used interchangeably herein.

The term “cofactor” as used herein is a broad term, and is to be given its ordinary and customary meaning to a person of ordinary skill in the art (and is not to be limited to a special or customized meaning), and refers without limitation to one or more substances whose presence contributes to or is required for analyte-related activity of an enzyme. Analyte-related activity can include, but is not limited to, any one of or a combination of binding, electron transfer, and chemical transformation. Cofactors are inclusive of coenzymes, non-protein chemical compounds, metal ions and/or metal organic complexes. Coenzymes are inclusive of prosthetic groups and co-substrates.

The term “continuous” as used herein is a broad term, and is to be given its ordinary and customary meaning to a person of ordinary skill in the art (and is not to be limited to a special or customized meaning), and refers without limitation to an uninterrupted or unbroken portion, domain, coating, or layer.

The phrases “continuous analyte sensing” and “continuous multi-analyte sensing” as used herein are broad phrases, and are to be given their ordinary and customary meaning to a person of ordinary skill in the art (and is not to be limited to a special or customized meaning), and refers without limitation to the period in which monitoring of analyte concentration is continuously, continually, and/or intermittently (but regularly) performed, for example, from about every second or less to about one week or more. In further examples, monitoring of analyte concentration is performed from about every 2, 3, 5, 7, 10, 15, 20, 25, 30, 35, 40, 45, 50, 55, or 60 seconds to about every 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50 or 9.75 minutes. In further examples, monitoring of analyte concentration is performed from about 10, 20, 30, 40 or 50 minutes to about every 1, 2, 3, 4, 5, 6, 7 or 8 hours. In further examples, monitoring of analyte concentration is performed from about every 8 hours to about every 12, 16, 20, or 24 hours. In further examples, monitoring of analyte concentration is performed from about every day to about every 1.5, 2, 3, 4, 5, 6, or 7 days. In further examples, monitoring of analyte concentration is performed from about every week to about every 1.5, 2, 3 or more weeks.

The term “coaxial” as used herein is to be construed broadly to include sensor architectures having elements aligned along a shared axis around a core that can be configured to have a circular, elliptical, triangular, polygonal, or other cross-section such elements can include electrodes, insulating layers, or other elements that can be positioned circumferentially around the core layer, such as a core electrode or core polymer wire.

The term “coupled” as used herein is a broad term, and is to be given its ordinary and customary meaning to a person of ordinary skill in the art (and is not to be limited to a special or customized meaning), and refers without limitation to two or more system elements or components that are configured to be at least one of electrically, mechanically, thermally, operably, chemically or otherwise attached. For example, an element is “coupled” if the element is covalently, communicatively, electrostatically, thermally connected, mechanically connected, magnetically connected, or ionically associated with, or physically entrapped, adsorbed to or absorbed by another element. Similarly, the phrases “operably connected”, “operably linked”, and “operably coupled” as used herein may refer to one or more components linked to another component(s) in a manner that facilitates transmission of at least one signal between the components. In some examples, components are part of the same structure and/or integral with one another as in covalently, electrostatically, mechanically, thermally, magnetically, ionically associated with, or physically entrapped, or absorbed (i.e. “directly coupled” as in no intervening element(s)). In other examples, components are connected via remote means. For example, one or more electrodes can be used to detect an analyte in a sample and convert that information into a signal; the signal can then be transmitted to an electronic circuit. In this example, the electrode is “operably linked” to the electronic circuit. The phrase “removably coupled” as used herein may refer to two or more system elements or components that are configured to be or have been electrically, mechanically, thermally, operably, chemically, or otherwise attached and detached without damaging any of the coupled elements or components. The phrase “permanently coupled” as used herein may refer to two or more system elements or components that are configured to be or have been electrically, mechanically, thermally, operably, chemically, or otherwise attached but cannot be uncoupled without damaging at least one of the coupled elements or components. covalently, electrostatically, ionically associated with, or physically entrapped, or absorbed

The term “discontinuous” as used herein is a broad term, and is to be given its ordinary and customary meaning to a person of ordinary skill in the art (and is not to be limited to a special or customized meaning), and refers without limitation to disconnected, interrupted, or separated portions, layers, coatings, or domains.

The term “distal” as used herein is a broad term, and is to be given its ordinary and customary meaning to a person of ordinary skill in the art (and is not to be limited to a special or customized meaning), and refers without limitation to a region spaced relatively far from a point of reference, such as an origin or a point of attachment.

The term “domain” as used herein is a broad term, and is to be given its ordinary and customary meaning to a person of ordinary skill in the art (and is not to be limited to a special or customized meaning), and refers without limitation to a region of a membrane system that can be a layer, a uniform or non-uniform gradient (for example, an anisotropic region of a membrane), or a portion of a membrane that is capable of sensing one, two, or more analytes. The domains discussed herein can be formed as a single layer, as two or more layers, as pairs of bi-layers, or as combinations thereof.

The term “electrochemically reactive surface” as used herein is a broad term, and is to be given its ordinary and customary meaning to a person of ordinary skill in the art (and is not to be limited to a special or customized meaning), and refers without limitation to the surface of an electrode where an electrochemical reaction takes place. In one example this reaction is faradaic and results in charge transfer between the surface and its environment. In one example, hydrogen peroxide produced by an enzyme-catalyzed reaction of an analyte being oxidized on the surface results in a measurable electronic current. For example, in the detection of glucose, glucose oxidase produces hydrogen peroxide (H₂O₂) as a byproduct. The H₂O₂ reacts with the surface of the working electrode to produce two protons (2H⁺), two electrons (2e⁻) and one molecule of oxygen (O₂), which produces the electronic current being detected. In a counter electrode, a reducible species, for example, O₂ is reduced at the electrode surface so as to balance the current generated by the working electrode.

The term “electrolysis” as used herein is a broad term, and is to be given its ordinary and customary meaning to a person of ordinary skill in the art (and is not to be limited to a special or customized meeting), and refers without limitation to electrooxidation or electroreduction (collectively, “redox”) of a compound, either directly or indirectly, by one or more enzymes, cofactors, or mediators.

The terms “indwelling,” “in dwelling,” “implanted,” or “implantable” as used herein are broad terms, and are to be given their ordinary and customary meaning to a person of ordinary skill in the art (and are not to be limited to a special or customized meaning), and refer without limitation to objects including sensors that are inserted, or configured to be inserted, subcutaneously (i.e. in the layer of fat between the skin and the muscle), intracutaneously (i.e. penetrating the stratum corneum and positioning within the epidermal or dermal strata of the skin), or transcutaneously (i.e. penetrating, entering, or passing through intact skin), which may result in a sensor that has an in vivo portion and an ex vivo portion. The term “indwelling” also encompasses an object which is configured to be inserted subcutaneously, intracutaneously, or transcutaneously, whether or not it has been inserted as such.

The terms “interferants” and “interfering species” as used herein are broad terms, and are to be given their ordinary and customary meaning to a person of ordinary skill in the art (and are not to be limited to a special or customized meaning), and refer without limitation to effects and/or species that interfere with the measurement of an analyte of interest in a sensor to produce a signal that does not accurately represent the analyte measurement. In one example of an electrochemical sensor, interfering species are compounds which produce a signal that is not analyte-specific due to a reaction on an electrochemically active surface.

The term “in vivo” as used herein is a broad term, and is to be given its ordinary and customary meaning to a person of ordinary skill in the art (and is not to be limited to a special or customized meaning), and without limitation is inclusive of the portion of a device (for example, a sensor) adapted for insertion into and/or existence within a living body of a host.

The term “ex vivo” as used herein is a broad term, and is to be given its ordinary and customary meaning to a person of ordinary skill in the art (and is not to be limited to a special or customized meaning), and without limitation is inclusive of a portion of a device (for example, a sensor) adapted to remain and/or exist outside of a living body of a host.

The term and phrase “mediator” and “redox mediator” as used herein are broad terms and phrases, and are to be given their ordinary and customary meaning to a person of ordinary skill in the art (and is not to be limited to a special or customized meaning), and refers without limitation to any chemical compound or collection of compounds capable of electron transfer, either directly, or indirectly, between an analyte, analyte precursor, analyte surrogate, analyte-reduced or analyte-oxidized enzyme, or cofactor, and an electrode surface held at a potential. In one example the mediator accepts electrons from, or transfer electrons to, one or more enzymes or cofactors, and/or exchanges electrons with the sensor system electrodes. In one example, mediators are transition-metal coordinated organic molecules which are capable of reversible oxidation and reduction reactions. In other examples, mediators may be organic molecules or metals which are capable of reversible oxidation and reduction reactions.

The term “membrane” as used herein is a broad term, and is to be given its ordinary and customary meaning to a person of ordinary skill in the art (and is not to be limited to a special or customized meaning), and refers without limitation to a structure configured to perform functions including, but not limited to, protection of the exposed electrode surface from the biological environment, diffusion resistance (limitation) of the analyte, service as a matrix for a catalyst (e.g., one or more enzymes) for enabling an enzymatic reaction, limitation or blocking of interfering species, provision of hydrophilicity at the electrochemically reactive surfaces of the sensor interface, service as an interface between host tissue and the implantable device, modulation of host tissue response via drug (or other substance) release, and combinations thereof. When used herein, the terms “membrane” and “matrix” are meant to be interchangeable.

The phrase “membrane system” as used herein is a broad phrase, and is to be given its ordinary and customary meaning to a person of ordinary skill in the art (and is not to be limited to a special or customized meaning), and refers without limitation to a permeable or semi-permeable membrane that can be comprised of two or more domains, layers, or layers within a domain, and is typically constructed of materials of a few microns thickness or more, which is permeable to oxygen and is optionally permeable to, e.g., glucose or another analyte. In one example, the membrane system comprises an enzyme, which enables an analyte reaction to occur whereby a concentration of the analyte can be measured.

The term “planar” as used herein is to be interpreted broadly to describe sensor architecture having a substrate including at least a first surface and an opposing second surface, and for example, comprising a plurality of elements arranged on one or more surfaces or edges of the substrate. The plurality of elements can include conductive or insulating layers or elements configured to operate as a circuit. The plurality of elements may or may not be electrically or otherwise coupled. In one example, planar includes one or more edges separating the opposed surfaces.

The term “proximal” as used herein is a broad term, and is to be given its ordinary and customary meaning to a person of ordinary skill in the art (and is not to be limited to a special or customized meaning), and refers without limitation to the spatial relationship between various elements in comparison to a particular point of reference. For example, some examples of a device include a membrane system having a biointerface layer and an enzyme domain or layer. If the sensor is deemed to be the point of reference and the enzyme domain is positioned nearer to the sensor than the biointerface layer, then the enzyme domain is more proximal to the sensor than the biointerface layer.

The phrases “sensing portion,” “sensing membrane,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In one example, the sensing portion, sensing membrane, and/or sensing mechanism generally comprise an electrode configured to provide signals during electrochemically reactions with one or more membranes covering electrochemically reactive surface. In one example, such sensing portions, sensing membranes, and/or sensing mechanisms are capable of providing specific quantitative, semi-quantitative, qualitative, semi qualitative analytical signals using a biological recognition element combined with a transducing and/or detecting element.

During general operation of the analyte measuring device, biosensor, sensor, sensing region, sensing portion, or sensing mechanism, a biological sample, for example, blood or interstitial fluid, or a component thereof contacts, either directly, or after passage through one or more membranes, an enzyme, for example, glucose oxidase, DNA, RNA, or a protein or aptamer, for example, one or more periplasmic binding protein (PBP) or mutant or fusion protein thereof having one or more analyte binding regions, each region capable of specifically or reversibly binding to and/or reacting with at least one analyte. The interaction of the biological sample or component thereof with the analyte measuring device, biosensor, sensor, sensing region, sensing portion, or sensing mechanism results in transduction of a signal that permits a qualitative, semi-qualitative, quantitative, or semi-qualitative determination of the analyte level, for example, glucose, ketone, lactate, potassium, etc., in the biological sample.

In one example, the sensing region or sensing portion can comprise at least a portion of a conductive substrate or at least a portion of a conductive surface, for example, a wire (coaxial) or conductive trace or a substantially planar substrate including substantially planar trace(s), and a membrane. In one example, the sensing region or sensing portion can comprise a non-conductive body, a working electrode, a reference electrode, and a counter electrode (optional), forming an electrochemically reactive surface at one location on the body and an electronic connection at another location on the body, and a sensing membrane affixed to the body and covering the electrochemically reactive surface. In some examples, the sensing membrane further comprises an enzyme domain, for example, an enzyme domain, and an electrolyte phase, for example, a free-flowing liquid phase comprising an electrolyte-containing fluid described further below. The terms are broad enough to include the entire device, or only the sensing portion thereof (or something in between).

In another example, the sensing region can comprise one or more periplasmic binding protein (PBP) including mutant or fusion protein thereof, or aptamers having one or more analyte binding regions, each region capable of specifically and reversibly binding to at least one analyte. Alterations of the aptamer or mutations of the PBP can contribute to or alter one or more of the binding constants, long-term stability of the protein, including thermal stability, to bind the protein to a special encapsulation matrix, membrane or polymer, or to attach a detectable reporter group or “label” to indicate a change in the binding region or transduce a signal corresponding to the one or more analytes present in the biological fluid. Specific examples of changes in the binding region include, but are not limited to, hydrophobic/hydrophilic environmental changes, three-dimensional conformational changes, changes in the orientation of amino/nucleic acid side chains in the binding region of proteins, and redox states of the binding region. Such changes to the binding region provide for transduction of a detectable signal corresponding to the one or more analytes present in the biological fluid.

In one example, the sensing region determines the selectivity among one or more analytes, so that only the analyte which has to be measured leads to (transduces) a detectable signal. The selection may be based on any chemical or physical recognition of the analyte by the sensing region, where the chemical composition of the analyte is unchanged, or in which the sensing region causes or catalyzes a reaction of the analyte that changes the chemical composition of the analyte.

The term “sensitivity” as used herein is a broad term, and is to be given its ordinary and customary meaning to a person of ordinary skill in the art (and is not to be limited to a special or customized meaning), and refers without limitation to an amount of signal (e.g., in the form of electrical current and/or voltage) produced by a predetermined amount (unit) of the measured analyte. For example, in one example, a sensor has a sensitivity (or slope) of from about 1 to about 100 picoAmps of current for every 1 mg/dL of analyte.

The phrases “signal medium” or “transmission medium” shall be taken to include any form of modulated data signal, carrier wave, and so forth. The phrase “modulated data signal” means a signal that has one or more of its characteristics set or changed in such a matter as to encode information in the signal.

The terms “transducing” or “transduction” and their grammatical equivalents as are used herein are broad terms, and are to be given their ordinary and customary meaning to a person of ordinary skill in the art (and is not to be limited to a special or customized meaning), and refer without limitation to optical, electrical, electrochemical, acoustical/mechanical, or colorimetrical technologies and methods. Electrochemical properties include current and/or voltage, inductance, capacitance, impedance, transconductance, and potential. Optical properties include absorbance, fluorescence/phosphorescence, fluorescence/phosphorescence decay rate, wavelength shift, dual wave phase modulation, bio/chemiluminescence, reflectance, light scattering, and refractive index. For example, the sensing region transduces the recognition of analytes into a semi-quantitative or quantitative signal.

As used herein, the phrase “transducing element” as used herein is a broad phrase, and are to be given their ordinary and customary meaning to a person of ordinary skill in the art (and is not to be limited to a special or customized meaning), and refers without limitation to analyte recognition moieties capable of facilitating, directly or indirectly, with detectable signal transduction corresponding to the presence and/or concentration of the recognized analyte. In one example, a transducing element is one or more enzymes, one or more aptamers, one or more ionophores, one or more capture antibodies, one or more proteins, one or more biological cells, one or more oligonucleotides, and/or one or more DNA or RNA moieties. Transcutaneous continuous multi-analyte sensors can be used in vivo over various lengths of time. The continuous multi-analyte sensor systems discussed herein can be transcutaneous devices, in that a portion of the device may be inserted through the host's skin and into the underlying soft tissue while a portion of the device remains on the surface of the host's skin. In one aspect, in order to overcome the problems associated with noise or other sensor function in the short-term, one example employs materials that promote formation of a fluid pocket around the sensor, for example architectures such as a porous biointerface membrane or matrices that create a space between the sensor and the surrounding tissue. In some examples, a sensor is provided with a spacer adapted to provide a fluid pocket between the sensor and the host's tissue. It is believed that this spacer, for example a biointerface material, matrix, structure, and the like as described in more detail elsewhere herein, provides for oxygen and/or glucose transport to the sensor.

### Membrane Systems

Membrane systems disclosed herein are suitable for use with implantable devices in contact with a biological fluid. For example, the membrane systems can be utilized with implantable devices, such as devices for monitoring and determining analyte levels in a biological fluid, for example, devices for monitoring glucose levels for individuals having diabetes. In some examples, the analyte-measuring device is a continuous device. The analyte-measuring device can employ any suitable sensing element to provide the raw signal, including but not limited to those involving enzymatic, chemical, physical, electrochemical, spectrophotometric, amperometric, potentiometric, polarimetric, calorimetric, radiometric, immunochemical, or like elements.

Suitable membrane systems for the aforementioned multi-analyte systems and devices can include, for example, membrane systems disclosed in U.S. Pat. Nos. 6,015,572, 5,964,745, and 6,083,523, which are incorporated herein by reference in their entireties for their teachings of membrane systems.

In general, the membrane system includes a plurality of domains, for example, an electrode domain, an interference domain, an enzyme domain, a resistance domain, and a biointerface domain. The membrane system can be deposited on the exposed electroactive surfaces using known thin film techniques (for example, vapor deposition, spraying, electrodepositing, dipping, brush coating, film coating, drop-let coating, and the like). Additional steps may be applied following the membrane material deposition, for example, drying, annealing, and curing (for example, UV curing, thermal curing, moisture curing, radiation curing, and the like) to enhance certain properties such as mechanical properties, signal stability, and selectivity. In a typical process, upon deposition of the resistance domain membrane, a biointerface/drug releasing layer having a “dry film” thickness of from about 0.05 micron (μm), or less, to about 1, 2, 3, 4, 5, 6, 7, 8, 9, 10, 11, 12, 13, 14, 15, or 16 μm is formed. “Dry film” thickness refers to the thickness of a cured film cast from a coating formulation by standard coating techniques.

In certain examples, the biointerface/drug releasing layer is formed of a biointerface polymer, wherein the biointerface polymer comprises one or more membrane domains comprising polyurethane and/or polyurea segments and one or more zwitterionic repeating units. In some examples, the biointerface/drug releasing layer coatings are formed of a polyurethane urea having carboxyl betaine groups incorporated in the polymer and non-ionic hydrophilic polyethylene oxide segments, wherein the polyurethane urea polymer is dissolved in organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like. Depending on the final thickness of the biointerface/drug releasing layer and solution viscosity (as related to the percent of polymer solid), the coating can be applied in a single step or multiple repeated steps of the chosen process such as dipping to build the desired thickness. Yet in other examples, the bioprotective polymers are formed of a polyurethane urea having carboxylic acid groups and carboxyl betaine groups incorporated in the polymer and non-ionic hydrophilic polyethylene oxide segments, wherein the polyurethane urea polymer is dissolved in an organic or non-organic solvent system in a coating formulation, and is crosslinked with an a carbodiimide (e.g., 1-ethyl-3-(3-dimethylaminopropyl)carbodiimide (EDC)) and cured at a moderate temperature of about 50° C.

In other examples, the biointerface/drug releasing layer coatings are formed of a polyurethane urea having sulfobetaine groups incorporated in the polymer and non-ionic hydrophilic polyethylene oxide segments, wherein the polyurethane urea polymer is dissolved in an organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like. Depending on the final thickness of the biointerface/drug releasing layer and solution viscosity (as related to the percent of polymer solid), the coating can be applied in a single step or multiple repeated steps of the chosen process such as dipping to build the desired thickness. Yet in other examples, the biointerface polymers are formed of a polyurethane urea having unsaturated hydrocarbon groups and sulfobetaine groups incorporated in the polymer and non-ionic hydrophilic polyethylene oxide segments, wherein the polyurethane urea polymer is dissolved in an organic or non-organic solvent system in a coating formulation, and is crosslinked in the presence of initiators with heat or irradiation including UV, LED light, electron beam, and the like, and cured at a moderate temperature of about 50° C. Examples of unsaturated hydrocarbon includes allyl groups, vinyl groups, acrylate, methacrylate, alkenes, alkynes, and the like.

In some examples, tethers are used. A tether is a polymer or chemical moiety which does not participate in the (electro)chemical reactions involved in sensing, but forms chemical bonds with the (electro)chemically active components of the membrane. In some examples these bonds are covalent. In one example, a tether may be formed in solution prior to one or more interlayers of a membrane being formed, where the tether bonds two (electro)chemically active components directly to one another or alternately, the tether(s) bond (electro)chemically active component(s) to polymeric backbone structures. In another example, (electro)chemically active components are comixed along with crosslinker(s) with tunable lengths (and optionally polymers) and the tethering reaction occurs as in situ crosslinking. Tethering may be employed to maintain a predetermined number of degrees of freedom of NAD(P)H for effective enzyme catalysis, where “effective” enzyme catalysis causes the analyte sensor to continuously monitor one or more analytes for a period of from about 5 days to about 15 days or more.

### Membrane Fabrication

Polymers can be processed by solution-based techniques such as spraying, dipping, casting, electrospinning, vapor deposition, spin coating, coating, and the like. Water-based polymer emulsions can be fabricated to form membranes by methods similar to those used for solvent-based materials. In both cases the evaporation of a volatile liquid (e.g., organic solvent or water) leaves behind a film of the polymer. Cross-linking of the deposited film or layer can be performed through the use of multi-functional reactive ingredients by a number of methods. The liquid system can cure by heat, moisture, high-energy radiation, ultraviolet light, or by completing the reaction, which produces the final polymer in a mold or on a substrate to be coated.

In some examples, the wetting property of the membrane (and by extension the extent of sensor drift exhibited by the sensor) can be adjusted and/or controlled by creating covalent cross-links between surface-active group-containing polymers, functional-group containing polymers, polymers with zwitterionic groups (or precursors or derivatives thereof), and combinations thereof. Cross-linking can have a substantial effect on film structure, which in turn can affect the film's surface wetting properties. Crosslinking can also affect the film's tensile strength, mechanical strength, water absorption rate and other properties.

Cross-linked polymers can have different cross-linking densities. In certain examples, cross-linkers are used to promote cross-linking between layers. In other examples, in replacement of (or in addition to) the cross-linking techniques described above, heat is used to form cross-linking. For example, in some examples, imide and amide bonds can be formed between two polymers as a result of high temperature. In some examples, photo cross-linking is performed to form covalent bonds between the polycationic layers(s) and polyanionic layer(s). One major advantage to photo-cross-linking is that it offers the possibility of patterning. In certain examples, patterning using photo-cross linking is performed to modify the film structure and thus to adjust the wetting property of the membranes and membrane systems, as discussed herein.

Polymers with domains or segments that are functionalized to permit cross-linking can be made by methods at least as discussed herein. For example, polyurethaneurea polymers with aromatic or aliphatic segments having electrophilic functional groups (e.g., carbonyl, aldehyde, anhydride, ester, amide, isocyano, epoxy, allyl, or halo groups) can be crosslinked with a crosslinking agent that has multiple nucleophilic groups (e.g., hydroxyl, amine, urea, urethane, or thiol groups). In further examples, polyurethaneurea polymers having aromatic or aliphatic segments having nucleophilic functional groups can be crosslinked with a crosslinking agent that has multiple electrophilic groups. Still further, polyurethaneurea polymers having hydrophilic segments having nucleophilic or electrophilic functional groups can be crosslinked with a crosslinking agent that has multiple electrophilic or nucleophilic groups. Unsaturated functional groups on the polyurethane urea can also be used for crosslinking by reacting with multivalent free radical agents. Non-limiting examples of suitable cross-linking agents include isocyanate, carbodiimide, glutaraldehyde, aziridine, silane, or other aldehydes, epoxy, acrylates, free-radical based agents, ethylene glycol diglycidyl ether (EGDE), poly(ethylene glycol) diglycidyl ether (PEGDE), or dicumyl peroxide (DCP). In one example, from about 0.1% to about 15% w/w of cross-linking agent is added relative to the total dry weights of cross-linking agent and polymers added when blending the ingredients. In another example, about 1% to about 10% w/w of cross-linking agent is added relative to the total dry weights of cross-linking agent and polymers added when blending the ingredients. In yet another example, about 5% to about 15% w/w of cross-linking agent is added relative to the total dry weights of cross-linking agent and polymers added when blending the ingredients. During the curing process, substantially all of the cross-linking agent is believed to react, leaving substantially no detectable unreacted cross-linking agent in the final film.

Polymers disclosed herein can be formulated into mixtures that can be drawn into a film or applied to a surface using methods such as spraying, self-assembling monolayers (SAMs), painting, dip coating, vapor depositing, molding, 3-D printing, lithographic techniques (e.g., photolithograph), micro- and nano-pipetting printing techniques, silk-screen printing, etc.). The mixture can then be cured under high temperature (e.g., from about 30° C. to about 150° C.). Other suitable curing methods can include ultraviolet, e-beam, or gamma radiation, for example.

In some circumstances, using continuous multianalyte monitoring systems including sensor(s) configured with bioprotective and/or drug releasing membranes, it is believed that that foreign body response is the dominant event surrounding extended implantation of an implanted device and can be managed or manipulated to support rather than hinder or block analyte transport. In another aspect, in order to extend the lifetime of the sensor, one example employs materials that promote vascularized tissue ingrowth, for example within a porous biointerface membrane. For example, tissue in-growth into a porous biointerface material surrounding a sensor may promote sensor function over extended periods of time (e.g., weeks, months, or years). It has been observed that in-growth and formation of a tissue bed can take up to 3 weeks. Tissue ingrowth and tissue bed formation is believed to be part of the foreign body response. As will be discussed herein, the foreign body response can be manipulated by the use of porous bioprotective materials that surround the sensor and promote ingrowth of tissue and microvasculature over time.

Accordingly, a sensor as discussed in examples herein may include a biointerface layer. The biointerface layer, like the drug releasing layer, may include, but is not limited to, for example, porous biointerface materials including a solid portion and interconnected cavities, all of which are described in more detail elsewhere herein. The biointerface layer can be employed to improve sensor function in the long term (e.g., after tissue ingrowth).

Accordingly, a sensor as discussed in examples herein may include a drug releasing membrane at least partially functioning as or in combination with a biointerface membrane. The drug releasing membrane may include, for example, materials including a hard-soft segment polymer with hydrophilic and optionally hydrophobic domains, all of which are described in more detail elsewhere herein, can be employed to improve sensor function in the long term (e.g., after tissue ingrowth). In one example, the materials including a hard-soft segment polymer with hydrophilic and optionally hydrophobic domains are configured to release a combination of a derivative form of dexamethasone or dexamethasone acetate with dexamethasone such that one or more different rates of release of the anti-inflammatory is achieved and the useful life of the sensor is extended. Other suitable drug releasing membranes of the present disclosure can be selected from silicone polymers, polytetrafluoroethylene, expanded polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polyvinyl alcohol (PVA), poly vinyl acetate, ethylene vinyl acetate (EVA), polybutylene terephthalate (PBT), polymethylmethacrylate (PMMA), polyether ether ketone (PEEK), polyamides, polyurethanes and copolymers and blends thereof, polyurethane urea polymers and copolymers and blends thereof, cellulosic polymers and copolymers and blends thereof, poly(ethylene oxide) and copolymers and blends thereof, poly(propylene oxide) and copolymers and blends thereof, polysulfones and block copolymers thereof including, for example, di-block, tri-block, alternating, random and graft copolymers cellulose, hydrogel polymers, poly(2-hydroxyethyl methacrylate, pHEMA) and copolymers and blends thereof, hydroxyethyl methacrylate, (HEMA) and copolymers and blends thereof, polyacrylonitrile-polyvinyl chloride (PAN-PVC) and copolymers and blends thereof, acrylic copolymers and copolymers and blends thereof, nylon and copolymers and blends thereof, polyvinyl difluoride, polyanhydrides, poly(l-lysine), poly(L-lactic acid), hydroxyethylmethacrylate and copolymers and blends thereof, and hydroxyapatite and copolymers and blends thereof.

### Exemplary Multi-Analyte Sensor Membrane Configurations

Continuous multi-analyte sensors with various membrane configurations suitable for facilitating signal transduction corresponding to analyte concentrations, either simultaneously, intermittently, and/or sequentially are provided. In one example, such sensors can be configured using a signal transducer, comprising one or more transducing elements (“TL”). Such continuous multi-analyte sensor can employ various transducing means, for example, amperometry, voltammetric, potentiometry, and impedimetric methods, among other techniques.

In one example, the transducing element comprises one or more membranes that can comprise one or more layers and or domains, each of the one or more layers or domains can independently comprise one or more signal transducers, e.g., enzymes, RNA, DNA, aptamers, binding proteins, etc. As used herein, transducing elements includes enzymes, ionophores, RNA, DNA, aptamers, binding proteins and are used interchangeably.

In one example, the transducing element is present in one or more membranes, layers, or domains formed over a sensing region. In one example, such sensors can be configured using one or more enzyme domains, e.g., membrane domains including enzyme domains, also referred to as EZ layers (“EZLs”), each enzyme domain may comprise one or more enzymes. Reference hereinafter to an “enzyme layer” is intended to include all or part of an enzyme domain, either of which can be all or part of a membrane system as discussed herein, for example, as a single layer, as two or more layers, as pairs of bi-layers, or as combinations thereof.

In one example, the continuous multi-analyte sensor uses one or more of the following analyte-substrate/enzyme pairs: for example, sarcosine oxidase in combination with creatinine amidohydrolase, creatine amidohydrolase being employed for the sensing of creatinine. Other examples of analytes/oxidase enzyme combinations that can be used in the sensing region include, for example, alcohol/alcohol oxidase, cholesterol/cholesterol oxidase, galactose:galactose/galactose oxidase, choline/choline oxidase, glutamate/glutamate oxidase, glycerol/glycerol-3phosphate oxidase (or glycerol oxidase), bilirubin/bilirubin oxidase, ascorbic/ascorbic acid oxidase, uric acid/uric acid oxidase, pyruvate/pyruvate oxidase, hypoxanthine:xanthine/xanthine oxidase, glucose/glucose oxidase, lactate/lactate oxidase, L-amino acid oxidase, and glycine/sarcosine oxidase. Other analyte-substrate/enzyme pairs can be used, including such analyte-substrate/enzyme pairs that comprise genetically altered enzymes, immobilized enzymes, mediator-wired enzymes, dimerized and/or fusion enzymes.

### NAD Based Multi-Analyte Sensor Platform

Nicotinamide adenine dinucleotide (NAD(P)⁺/NAD(P)H) is a coenzyme, e.g., a dinucleotide that consists of two nucleotides joined through their phosphate groups. One nucleotide contains an adenine nucleobase and the other nicotinamide. NAD exists in two forms, e.g., an oxidized form (NAD(P)+) and reduced form (NAD(P)H) (H=hydrogen). The reaction of NAD+ and NADH is reversible, thus, the coenzyme can continuously cycle between the NAD(P)⁺/and NAD(P)H forms essentially without being consumed.

In one example, one or more enzyme domains of the sensing region of the presently disclosed continuous multi-analyte sensor device comprise an amount of NAD+ or NADH for providing transduction of a detectable signal corresponding to the presence or concentration of one or more analytes. In one example, one or more enzyme domains of the sensing region of the presently disclosed continuous multi-analyte sensor device comprise an excess amount of NAD+ or NADH for providing extended transduction of a detectable signal corresponding to the presence or concentration of one or more analytes.

In one example, NAD, NADH, NAD+, NAD(P)+, ATP, flavin adenine dinucleotide (FAD), magnesium (Mg++), pyrroloquinoline quinone (PQQ), and functionalized derivatives thereof can be used in combination with one or more enzymes in the continuous multi-analyte sensor device. In one example, NAD, NADH, NAD+, NAD(P)+, ATP, flavin adenine dinucleotide (FAD), magnesium (Mg++), pyrroloquinoline quinone (PQQ), and functionalized derivatives are incorporated in the sensing region. In one example, NAD, NADH, NAD+, NAD(P)+, ATP, flavin adenine dinucleotide (FAD), magnesium (Mg++), pyrroloquinoline quinone (PQQ), and functionalized derivatives are dispersed or distributed in one or more membranes or domains of the sensing region.

In one aspect of the present disclosure, continuous sensing of one or more or two or more analytes using NAD+ dependent enzymes is provided in one or more membranes or domains of the sensing region. In one example, the membrane or domain provides retention and stable recycling of NAD+ as well as mechanisms for transducing NADH oxidation or NAD+ reduction into measurable current with amperometry. In one example, described below, continuous, sensing of multi-analytes, either reversibly bound or at least one of which are oxidized or reduced by NAD+ dependent enzymes, for example, ketones (beta-hydroxybutyrate dehydrogenase), glycerol (glycerol dehydrogenase), cortisol (11β-hydroxysteroid dehydrogenase), glucose (glucose dehydrogenase), alcohol (alcohol dehydrogenase), aldehydes (aldehyde dehydrogenase), and lactate (lactate dehydrogenase) is provided. In other examples, described below, membranes are provided that enable the continuous, on-body sensing of multiple analytes which utilize FAD-dependent dehydrogenases, such as fatty acids (Acyl-CoA dehydrogenase).

Exemplary configurations of one or more membranes or portions thereof are an arrangement for providing retention and recycling of NAD+ are provided. Thus, an electrode surface of a conductive wire (coaxial) or a planar conductive surface is coated with at least one layer comprising at least one enzyme as depicted in FIG. 7A. With reference to FIG. 7B, one or more optional layers may be positioned between the electrode surface and the one or more enzyme domains. For example, one or more interference domains (also referred to as “interferent blocking layer”) can be used to reduce or eliminate signal contribution from undesirable species present, or one or more electrodes (not shown) can used to assist with wetting, system equilibrium, and/or start up. As shown in FIGS. 7A-7B, one or more of the membranes provides a NAD+ reservoir domain providing a reservoir for NAD+. In one example, one or more interferent blocking membranes is used, and potentiostat is utilized to measure H2O2 production or O2 consumption of an enzyme such as or similar to NADH oxidase, the NAD+ reservoir and enzyme domain positions can be switched, to facilitate better consumption and slower unnecessary outward diffusion of excess NAD+. Exemplary sensor configurations can be found in U.S. Provisional Patent Application No. 63/321,340, “CONTINUOUS ANALYTE MONITORING SENSOR SYSTEMS AND METHODS OF USING THE SAME,” filed Mar. 18, 2022, and incorporated by reference in its entirety herein.

In one example, one or more mediators that are optimal for NADH oxidation are incorporated in the one or more electrode domains or enzyme domains. In one example, organic mediators, such as phenanthroline dione, or nitrosoanilines are used. In another example, metallo-organic mediators, such as ruthenium-phenanthroline-dione or osmium(bpy)₂Cl, polymers containing covalently coupled organic mediators or organometallic coordinated mediators polymers for example polyvinylimidizole-Os(bpy)₂Cl, or polyvinylpyridine-organometallic coordinated mediators (including ruthenium-phenanthroline dione) are used. Other mediators can be used as discussed further below.

In humans, serum levels of beta-hydroxybutyrate (BHB) are usually in the low micromolar range but can rise up to about 6-8 mM. Serum levels of BHB can reach 1-2 mM after intense exercise or consistent levels above 2 mM are reached with a ketogenic diet that is almost devoid of carbohydrates. Other ketones are present in serum, such as acetoacetate and acetone, however, most of the dynamic range in ketone levels is in the form of BHB. Thus, monitoring of BHB, e.g., continuous monitoring is useful for providing health information to a user or health care provider.

Another example of a continuous ketone analyte detection configuration employing electrode-associated mediator-coupled diaphorase/NAD+/dehydrogenase is depicted below:

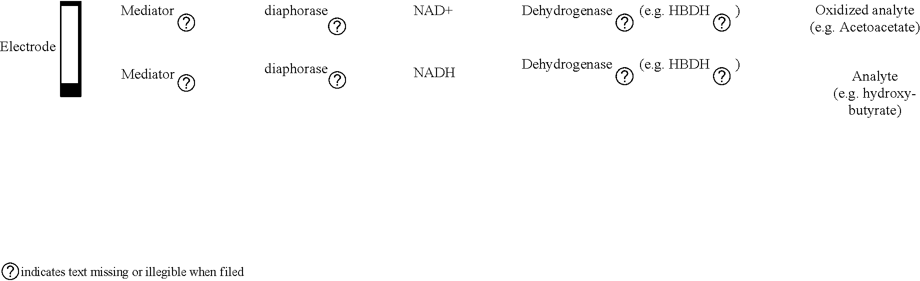

In one example, the diaphorase is electrically coupled to the electrode with organometallic coordinated mediator polymer. In another example, the diaphorase is covalently coupled to the electrode with an organometallic coordinated mediator polymer. Alternatively, multiple enzyme domains can be used in an enzyme layer, for example, separating the electrode-associated diaphorase (closest to the electrode surface) from the more distal adjacent NAD+ or the dehydrogenase enzyme, to essentially decouple NADH oxidation from analyte (ketone) oxidation. Alternatively, NAD+ can be more proximal to the electrode surface than an adjacent enzyme domain comprising the dehydrogenase enzyme. In one example, the NAD+ and/or HBDH are present in the same or different enzyme domain, and either can be immobilized, for example, using amine reactive crosslinker (e.g., glutaraldehyde, epoxides, NHS esters, imidoesters). In one example, the NAD+ is coupled to a polymer and is present in the same or different enzyme domain as HBDH. In one example, the molecular weight of NAD+ is increased to prevent or eliminate migration from the sensing region, for example the NAD+ is dimerized using its C6 terminal amine with any amine-reactive crosslinker. In one example, NAD+ may be covalently coupled to an aspect of the enzyme domain having a higher molecular weight than the NAD+ which may improve a stability profile of the NAD+, improving the ability to retain and/or immobilize the NAD+ in the enzyme domain. For example, dextran-NAD.

In one example, the sensing region comprises one or more NADH:acceptor oxidoreductases and one or more NAD-dependent dehydrogenases. In one example, sensing region comprises one or more NADH:acceptor oxidoreductases and one or more NAD(P)-dependent dehydrogenases with NAD(P)+ or NAD(P)H as cofactors present in sensing region. In one example, the sensing region comprises an amount of diaphorase.

In one example, a ketone sensing configuration suitable for combination with another analyte sensing configuration is provided. Thus, an EZL layer of about 1-20 um thick is prepared by presenting a EZL solution composition in 10 mM HEPES in water having about 20 uL 500 mg/mL HBDH, about 20 uL [500 mg/mL NAD(P)H, 200 mg/mL polyethylene glycol-diglycol ether (PEG-DGE) of about 400 MW], about 20 uL 500 mg/mL diaphorase, about 40 uL 250 mg/mL poly vinyl imidazole-osmium bis(2,2′-bipyridine)chloride (PVI-Os(bpy)2Cl) to a substrate such as a working electrode, so as to provide, after drying, about 15-40% by weight HBDH, about 5-30% diaphorase about 5-30% NAD(P)H, about 10-50% PVI-Os(bpy)2Cl and about 1-12% PEG-DGE (400 MW). The substrates discussed herein that may include working electrodes may be formed from gold, platinum, palladium, rhodium, iridium, titanium, tantalum, chromium, and/or alloys or combinations thereof, or carbon (e.g., graphite, glassy carbon, carbon nanotubes, graphene, or doped diamond, as well combinations thereof.

To the above enzyme domain was contacted a resistance domain, also referred to as a resistance layer (“RL”). In one example, the RL comprises about 55-100% PVP, and about 0.1-45% PEG-DGE. In another example, the RL comprises about 75-100% PVP, and about 0.3-25% PEG-DGE. In yet another example, the RL comprises about 85-100% PVP, and about 0.5-15% PEG-DGE. In yet another example, the RL comprises essentially 100% PVP.

The exemplary continuous ketone sensor as depicted in FIGS. 7A-7B comprising NAD(P)H reservoir domain is configured so that NAD(P)H is not rate-limiting in any of the enzyme domains of the sensing region. In one example, the loading of NAD(P)H in the NAD(P)H reservoir domain is greater than about 20%, 30%, 40% or 50% w/w. The one or more of the membranes or portions of one or more membrane domains (hereinafter also referred to as “membranes”) may also contain a polymer or protein binder, such as zwitterionic polyurethane, and/or albumin. Alternatively, in addition to NAD(P)H, the membrane may contain one or more analyte specific enzymes (e.g. HBDH, glycerol dehydrogenase, etc.), so that optionally, the NAD(P)H reservoir membrane also provides a catalytic function. In one example, the NAD(P)H is dispersed or distributed in or with a polymer (or protein), and may be crosslinked to an extent that still allows adequate enzyme/cofactor functionality and/or reduced NAD(P)H flux within the domain.

In one example, NADH oxidase enzyme alone or in combination with superoxide dismutase (SOD) is used in the one or more membranes of the sensing region. In one example, an amount of superoxide dismutase (SOD) is used that is capable of scavenging some or most of one or more free radicals generated by NADH oxidase. In one example, NADH oxidase enzyme alone or in combination with superoxide dismutase (SOD) is used in combination with NAD(P)H and/or a functionalized polymer with NAD(P)H immobilized onto the polymer from a C6 terminal amine in the one or more membranes of the sensing region.

In one example, the NAD(P)H is immobilized to an extent that maintains NAD(P)H catalytic functionality. In one example, dimerized NAD(P)H is used to entrap NAD(P)H within one or more membranes by crosslinking their respective C6 terminal amine together with appropriate amine-reactive crosslinker such as glutaraldehyde or PEG-DGE.

The aforementioned continuous ketone sensor configurations can be adapted to other analytes or used in combination with other sensor configurations. For example, analyte(s)-dehydrogenase enzyme combinations can be used in any of the membranes of the sensing region include; glycerol (glycerol dehydrogenase); cortisol (11β-hydroxysteroid dehydrogenase); glucose (glucose dehydrogenase); alcohol (alcohol dehydrogenase); aldehydes (aldehyde dehydrogenase); and lactate (lactate dehydrogenase).

In one example, a semipermeable membrane is used in the sensing region or adjacent thereto or adjacent to one or more membranes of the sensing region so as to attenuate the flux of at least one analyte or chemical species. In one example, the semipermeable membrane attenuates the flux of at least one analyte or chemical species so as to provide a linear response from a transduced signal. In another example, the semipermeable membrane prevents or eliminates the flux of NAD(P)H out of the sensing region or any membrane or domain. In one example, the semipermeable membrane can be an ion selective membrane selective for an ion analyte of interest, such as ammonium ion.

In another example, a continuous multi-analyte sensor configuration comprising one or more enzymes and/or at least one cofactor was prepared. FIG. 7C depicts this exemplary configuration, of an enzyme domain 750 comprising an enzyme (Enzyme) with an amount of cofactor (Cofactor) that is positioned proximal to at least a portion of a working electrode (“WE”) surface, where the WE comprises an electrochemically reactive surface. In one example, a second membrane 751 comprising an amount of cofactor is positioned adjacent the first enzyme domain. The amount of cofactor in the second membrane can provide an excess for the enzyme, e.g., to extend sensor life. One or more resistance domains 752 (“RL”) are positioned adjacent the second membrane (or can be between the membranes). The RL can be configured to block diffusion of cofactor from the second membrane. Electron transfer from the cofactor to the WE transduces a signal that corresponds directly or indirectly to an analyte concentration.

FIG. 7D depicts an alternative enzyme domain configuration comprising a first membrane 751 with an amount of cofactor that is positioned more proximal to at least a portion of a WE surface. Enzyme domain 750 comprising an amount of enzyme is positioned adjacent the first membrane.

In the membrane configurations depicted in FIGS. 7C-7D, production of an electrochemically active species in the enzyme domain diffuses to the WE surface and transduces a signal that corresponds directly or indirectly to an analyte concentration. In some examples, the electrochemically active species comprises hydrogen peroxide. For sensor configurations that include a cofactor, the cofactor from the first layer can diffuse to the enzyme domain to extend sensor life, for example, by regenerating the cofactor. For other sensor configurations, the cofactor can be optionally included to improve performance attributes, such as stability. For example, a continuous ketone sensor can comprise NAD(P)H and a divalent metal cation, such as Mg⁺². One or more resistance domains RL can be positioned adjacent the second membrane (or can be between the layers). The RL can be configured to block diffusion of cofactor from the second membrane and/or interferents from reaching the WE surface. Other configurations can be used in the aforementioned configuration, such as electrode, resistance, bio-interfacing, and drug releasing membranes, layers or domains. In other examples, continuous analyte sensors including one or more cofactors that contribute to sensor performance.

FIG. 7E depicts another continuous multi-analyte membrane configuration, where {beta}-hydroxybutyrate dehydrogenase BHBDH in a first enzyme domain 753 is positioned proximate to a working electrode WE and second enzyme domain 754, for example, comprising alcohol dehydrogenase (ADH) and NADH is positioned adjacent the first enzyme domain. One or more resistance domains RL 752 may be deployed adjacent to the second enzyme domain 754. In this configuration, the presence of the combination of alcohol and ketone in serum works collectively to provide a transduced signal corresponding to at least one of the analyte concentrations, for example, ketone. Thus, as the NADH present in the more distal second enzyme domain consumes alcohol present in the serum environment, NADH is oxidized to NAD(P)H that diffuses into the first membrane layer to provide electron transfer of the BHBDH catalysis of acetoacetate ketone and transduction of a detectable signal corresponding to the concentration of the ketone. In one example, an enzyme can be configured for reverse catalysis and can create a substrate used for catalysis of another enzyme present, either in the same or different layer or domain. Other configurations can be used in the aforementioned configuration, such as electrode, resistance, bio-interfacing, and drug releasing membranes, layers, or domains. Thus, a first enzyme domain that is more distal from the WE than a second enzyme domain may be configured to generate a cofactor or other element to act as a reactant (and/or a reactant substrate) for the second enzyme domain to detect the one or more target analytes.

### Alcohol Sensor Configurations

In one example, a continuous alcohol (e.g., ethanol) sensor device configuration is provided. In one example, one or more enzyme domains comprising alcohol oxidase (AOX) is provided and the presence and/or amount of alcohol is transduced by creation of hydrogen peroxide, alone or in combination with oxygen consumption or with another substrate-oxidase enzyme system, e.g., glucose-glucose oxidase, in which hydrogen peroxide and or oxygen and/or glucose can be detected and/or measured qualitatively or quantitatively, using amperometry.

In one example, the sensing region for the aforementioned enzyme substrate-oxidase enzyme configurations has one or more enzyme domains comprises one or more electrodes. In one example, the sensing region for the aforementioned enzyme substrate-oxidase enzyme configurations has one or more enzyme domains, with or without the one or more electrodes, further comprises one or interference blocking membranes (e.g. permselective membranes, charge exclusion membranes) to attenuate one or more interferents from diffusing through the membrane to the working electrode. In one example, the sensing region for the aforementioned substrate-oxidase enzyme configurations has one or more enzyme domains, with or without the one or more electrodes, and further comprises one or resistance domains with or without the one or more interference blocking membranes to attenuate one or more analytes or enzyme substrates. In one example, the sensing region for the aforementioned substrate-oxidase enzyme configurations has one or more enzyme domains, with or without the one or more electrodes, one or more resistance domains with or without the one or more interference blocking membranes further comprises one or biointerface membranes and/or drug releasing membranes, independently, to attenuate one or more analytes or enzyme substrates and attenuate the immune response of the host after insertion.

In one example, the one or more interference blocking membranes are deposited adjacent the working electrode and/or the electrode surface. In one example, the one or interference blocking membranes are directly deposited adjacent the working electrode and/or the electrode surface. In one example, the one or interference blocking membranes are deposited between another layer or membrane or domain that is adjacent the working electrode or the electrode surface to attenuate one or all analytes diffusing thru the sensing region but for oxygen. Such membranes can be used to attenuate alcohol itself as well as attenuate other electrochemically actives species or other analytes that can otherwise interfere by producing a signal if they diffuse to the working electrode.

In one example, the working electrode used comprised platinum and the potential applied was about 0.5 volts.

In one example, sensing oxygen level changes electrochemically, for example in a Clark type electrode setup, or in a different configuration can be carried out, for example by coating the electrode with one or more membranes of one or more polymers, such as NAFION™. Based on changes of potential, oxygen concentration changes can be recorded, which correlate directly or indirectly with the concentrations of alcohol. When appropriately designed to obey stoichiometric behavior, the presence of a specific concentration of alcohol should cause a commensurate reduction in local oxygen in a direct (linear) relation with the concentration of alcohol. Accordingly, a multi-analyte sensor for both alcohol and oxygen can therefore be provided.

In another example, the above mentioned alcohol sensing configuration can include one or more secondary enzymes that react with a reaction product of the alcohol/alcohol oxidase catalysis, e.g., hydrogen peroxide, and provide for a oxidized form of the secondary enzyme that transduces an alcohol-dependent signal to the WE/RE at a lower potential than without the secondary enzyme. Thus, in one example, the alcohol/alcohol oxidase is used with a reduced form of a peroxidase, for example horse radish peroxidase. The alcohol/alcohol oxidase can be in same or different layer as the peroxidase, or they may be spatially separated distally from the electrode surface, for example, the alcohol/alcohol oxidase being more distal from the electrode surface and the peroxidase being more proximal to the electrode surface, or alternatively, the alcohol/alcohol oxidase being more proximal from the electrode surface and the peroxidase being more distal to the electrode surface. In one example, the alcohol/alcohol oxidase, being more distal from the electrode surface and the peroxidase, further includes any combination of electrode, interference, resistance, and biointerface membranes to optimize signal, durability, reduce drift, or extend end of use duration.

In another example, the above mentioned alcohol sensing configuration can include one or more mediators. In one example, the one or more mediators are present in, on, or about one or more electrodes or electrode surfaces and/or are deposited or otherwise associated with the surface of the working electrode (WE) or reference electrode (RE). In one example, the one or more mediators eliminate or reduce direct oxidation of interfering species that may reach the WE or RE. In one example, the one or more mediators provide a lowering of the operating potential of the WE/RE, for example, from about 0.6V to about 0.3V or less on a platinum electrode, which can reduce or eliminates oxidation of endogenous interfering species. Examples of one or mediators are provided below. Other electrodes, e.g., counter electrodes, can be employed.

In one example, other enzymes or additional components may be added to the polymer mixture(s) that constitute any part of the sensing region to increase the stability of the aforementioned sensor and/or reduce or eliminate the biproducts of the alcohol/alcohol oxidase reaction. Increasing stability includes storage or shelf life and/or operational stability (e.g., retention of enzyme activity during use). For example, byproducts of enzyme reactions may be undesirable for increased shelf life and/or operational stability, and may thus be desirable to reduce or remove. In one example, xanthine oxidase can be used to remove bi-products of one or more enzyme reactions.

In another example, a dehydrogenase enzyme is used with an oxidase for the detection of alcohol alone or in combination with oxygen. Thus, in one example, alcohol dehydrogenase is used to oxidize alcohol to aldehyde in the presence of reduced nicotinamide adenine dinucleotide (NAD(P)H) or reduced nicotinamide adenine dinucleotide phosphate (NAD(P)+). So as to provide a continuous source of NAD(P)H or NAD(P)+, NADH oxidase or NADPH oxidases is used to oxidize the NAD(P)H or NAD(P)+, with the consumption of oxygen. In another example, Diaphorase can be used instead of or in combination with NADH oxidase or NADPH oxidases. Alternatively, an excess amount of NAD(P)H can be incorporated into the one or more enzyme domains and/or the one or more electrodes in an amount so as to accommodate the intended duration of planned life of the sensor.

In the aforementioned dual enzyme configuration, a signal can be sensed either by: (1) an electrically coupled (e.g., “wired”) alcohol dehydrogenase (ADH), for example, using an electro-active hydrogel polymer comprising one or more mediators; or (2) oxygen electrochemical sensing to measure the oxygen consumption of the NADH oxidase. In an alternative example, the co-factor NAD(P)H or NAD(P)+ may be coupled to a polymer, such as dextran, the polymer immobilized in the enzyme domain along with ADH. This provides for retention of the co-factor and availability thereof for the active site of ADH. In the above example, any combination of electrode, interference, resistance, and biointerface membranes can be used to optimize signal, durability, reduce drift, or extend end of use duration. In one example, electrical coupling, for example, directly or indirectly, via a covalent or ionic bond, to at least a portion of a transducing element, such as an aptamer, an enzyme or cofactor and at least a portion of the electrode surface is provided. A chemical moiety capable of assisting with electron transfer from the enzyme or cofactor to the electrode surface can be used and includes one or more mediators as described below.

In one example, any one of the aforementioned continuous alcohol sensor configurations are combined with any one of the aforementioned continuous ketone monitoring configurations to provide a continuous multi-analyte sensor device as further described below. In one example a continuous glucose monitoring configuration combined with any one of the aforementioned continuous alcohol sensor configurations and any one of the aforementioned continuous ketone monitoring configurations to provide a continuous multi-analyte sensor device as further described below.

### Uric Acid Sensor Configurations

In another example, a continuous uric acid sensor device configuration is provided. Thus, in one example, uric acid oxidase (UOX) can be included in one or more enzyme domains and positioned adjacent the working electrode surface. The catalysis of the uric acid using UOX, produces hydrogen peroxide which can be detected using, among other techniques, amperometry, voltammetric and impedimetric methods. In one example, to reduce or eliminate the interference from direct oxidation of uric acid on the electrode surface, one or more electrode, interference, and/or resistance domains can be deposited on at least a portion of the working electrode surface. Such membranes can be used to attenuate diffusion of uric acid as well as other analytes to the working electrode that can interfere with signal transduction.

In one alternative example, a uric acid continuous sensing device configuration comprises sensing oxygen level changes about the WE surface, e.g., for example, as in a Clark type electrode setup, or the one or more electrodes can comprise, independently, one or more different polymers such as NAFION™, polyzwitterion polymers, or polymeric mediator adjacent at least a portion of the electrode surface. In one example, the electrode surface with the one or more electrode domains provide for operation at a different or lower voltage to measure oxygen. Oxygen level and its changes in can be sensed, recorded, and correlated to the concentration of uric acid based using, for example, using conventional calibration methods.

In one example, alone or in combination with any of the aforementioned configurations, uric acid sensor configurations, so as to lower the potential at the WE for signal transduction of uric acid, one or more coatings can be deposited on the WE surface. The one or more coatings may be deposited or otherwise formed on the WE surface and/or on other coatings formed thereon using various techniques including, but not limited to, dipping, electrodepositing, vapor deposition, spray coating, etc. In one example, the coated WE surface can provide for redox reactions, e.g., of hydrogen peroxide, at lower potentials (as compared to 0.6 V on platinum electrode surface without such a coating. Example of materials that can be coated or annealed onto the WE surface includes, but are not limited to Prussian Blue, Medola Blue, methylene blue, methylene green, methyl viologen, ferrocyanide, ferrocene, cobalt ion, and cobalt phthalocyanine, and the like.

In one example, one or more secondary enzymes, cofactors and/or mediators (electrically coupled or polymeric mediators) can be added to the enzyme domain with UOX to facilitate direct or indirect electron transfer to the WE. In such configurations, for example, regeneration of the initial oxidized form of secondary enzyme is reduced by the WE for signal transduction. In one example, the secondary enzyme is horse radish peroxidase (HRP).

### Choline Sensor Configurations

In one example continuous choline sensor device can be provided, for example, using choline oxidase enzyme that generates hydrogen peroxide with the oxidation of choline. Thus, in one example, at least one enzyme domain comprises choline oxidase (COX) adjacent at least one WE surface, optionally with one or more electrodes and/or interference membranes positioned in between the WE surface and the at least one enzyme domain. The catalysis of the choline using COX results in creation of hydrogen peroxide which can be detectable using, among other techniques, amperometry, voltammetric and impedimetric methods.

In one example, the aforementioned continuous choline sensor configuration is combined with any one of the aforementioned continuous alcohol sensor configurations, and continuous uric acid sensor configurations to provide a continuous multi-analyte sensor device as further described below. This continuous multi-analyte sensor device can further include continuous glucose monitoring capability. Other membranes can be used in the aforementioned continuous choline sensor configuration, such as electrode, resistance, bio-interfacing, and drug releasing membranes.

### Cholesterol Sensor Configurations

In one example, continuous cholesterol sensor configurations can be made using cholesterol oxidase (CHOX), in a manner similar to previously described sensors. Thus, one or more enzyme domains comprising CHOX can be positioned adjacent at least one WE surface. The catalysis of free cholesterol using CHOX results in creation of hydrogen peroxide which can be detectable using, among other techniques, amperometry, voltammetric and impedimetric methods.

An exemplary cholesterol sensor configuration using a platinum WE, where at least one interference membrane is positioned adjacent at least one WE surface, over which there is at least one enzyme domain comprising CHOX, over which is positioned at least one resistance domain to control diffusional characteristics was prepared.

The method described above and the cholesterol sensors described can measure free cholesterol, however, with modification, the configuration can measure more types of cholesterol as well as total cholesterol concentration. Measuring different types of cholesterol and total cholesterol is important, since due to low solubility of cholesterol in water significant amount of cholesterol is in unmodified and esterified forms. Thus, in one example, a total cholesterol sample is provided where a secondary enzyme is introduced into the at least one enzyme domain, for example, to provide the combination of cholesterol esterase with CHOX Cholesteryl ester, which essentially represents total cholesterols can be measured indirectly from signals transduced from cholesterol present and formed by the esterase.

In one example, the aforementioned continuous (total) cholesterol sensor configuration is combined with any one of the aforementioned continuous alcohol sensor configurations and/or continuous uric acid sensor configurations to provide a continuous multi-analyte sensor system as further described below. This continuous multi-analyte sensor device can further include continuous glucose monitoring capability. Other membrane configurations can be used in the aforementioned continuous cholesterol sensor configuration, such as one or more electrode domains, resistance domains, bio-interfacing domains, and drug releasing membranes.

### Bilirubin Sensor and Ascorbic Acid Sensor Configurations

In one example, continuous bilirubin and ascorbic acid sensors are provided. These sensors can employ bilirubin oxidase and ascorbate oxidase, respectively. However, unlike some oxidoreductase enzymes, the final product of the catalysis of analytes of bilirubin oxidase and ascorbate oxidase is water instead of hydrogen peroxide. Therefore, redox detection of hydrogen peroxide to correlate with bilirubin or ascorbic acid is not possible. However, these oxidase enzymes still consume oxygen for the catalysis, and the levels of oxygen consumption correlates with the levels of the target analyte present. Thus, bilirubin and ascorbic acid levels can be measured indirectly by electrochemically sensing oxygen level changes, as in a Clark type electrode setup, for example.

Alternatively, a different configuration for sensing bilirubin and ascorbic acid can be employed. For example, an electrode domain including one or more electrode domains comprising electron transfer agents, such as NAFION™, polyzwitterion polymers, or polymeric mediator can be coated on the electrode. Measured oxygen levels transduced from such enzyme domain configurations can be correlated with the concentrations of bilirubin and ascorbic acid levels. In one example, an electrode domain comprising one or more mediators electrically coupled to a working electrode can be employed and correlated to the levels of bilirubin and ascorbic acid levels.

In one example, the aforementioned continuous bilirubin and ascorbic acid sensor configurations can be combined with any one of the aforementioned continuous alcohol sensor configurations, continuous uric acid sensor configurations, continuous cholesterol sensor configurations to provide a continuous multi-analyte sensor device as further described below. This continuous multi-analyte sensor device can further include continuous glucose monitoring capability. Other membranes can be used in the aforementioned continuous bilirubin and ascorbic acid sensor configuration, such as electrode, resistance, bio-interfacing, and drug releasing membranes.

### One-Working-Electrode Configurations for Dual Analyte Detection

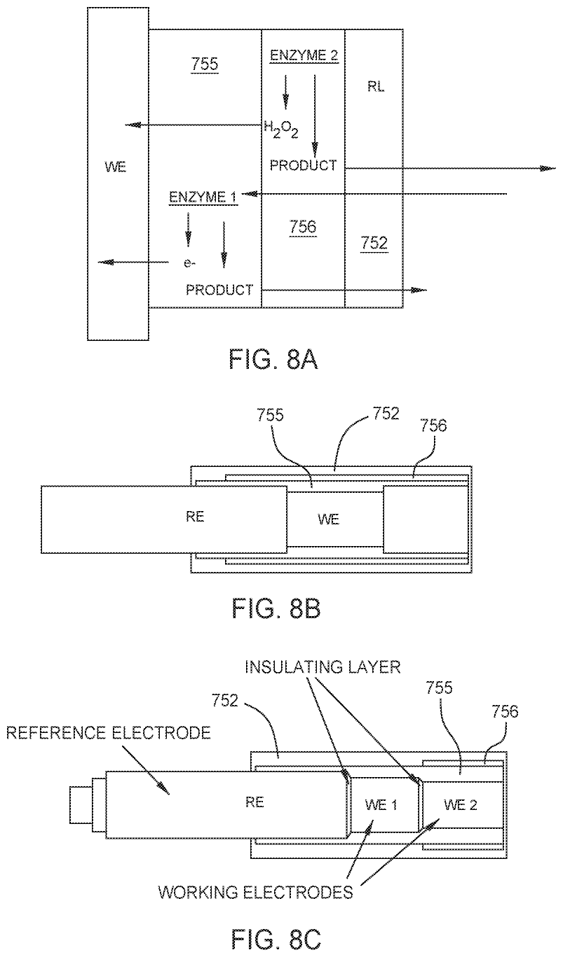

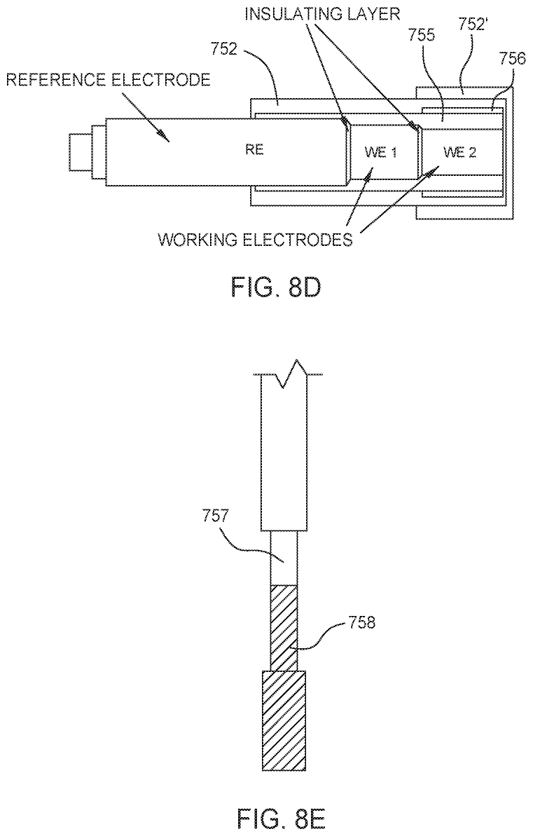

In one example, at least a dual enzyme domain configuration in which each layer contains one or more specific enzymes and optionally one or more cofactors is provided. In a broad sense, one example of a continuous multi-analyte sensor configuration is depicted in FIG. 8A where a first membrane 755 (EZL1) comprising at least one enzyme (Enzyme 1) of the at least two enzyme domain configuration is proximal to at least one surface of a WE. One or more analyte-substrate enzyme pairs with Enzyme 1 transduces at least one detectable signal to the WE surface by direct electron transfer or by mediated electron transfer that corresponds directly or indirectly to an analyte concentration. Second membrane 756 (EZL2) with at least one second enzyme (Enzyme 2) is positioned adjacent 755 ELZ1, and is generally more distal from WE than EZL1. One or more resistance domains (RL) 752 can be provided adjacent EZL2 756, and/or between EZL1 755 and EZL2 756. The different enzymes catalyze the transformation of the same analyte, but at least one enzyme in EZL2 756 provides hydrogen peroxide and the other at least one enzyme in EZL1 755 does not provide hydrogen peroxide. Accordingly, each measurable species (e.g., hydrogen peroxide and the other measurable species that is not hydrogen peroxide) generates a signal associated with its concentration.

For example, in the configuration shown in FIG. 8A, a first analyte diffuses through RL 752 and into EZL2 756 resulting in peroxide via interaction with Enzyme 2. Peroxide diffuses at least through EZL1 755 to WE and transduces a signal that corresponds directly or indirectly to the first analyte concentration. A second analyte, which is different from the first analyte, diffuses through RL 752 and EZL2 756 and interacts with Enzyme 1, which results in electron transfer to WE and transduces a signal that corresponds directly or indirectly to the second analyte concentration.

As shown in FIG. 8B, the above configuration is adapted to a conductive wire electrode construct, where at least two different enzyme-containing layers are constructed on the same WE with a single active surface. In one example, the single WE is a wire, with the active surface positioned about the longitudinal axis of the wire. In another example, the single WE is a conductive trace on a substrate, with the active surface positioned about the longitudinal axis of the trace. In one example, the active surface is substantially continuous about a longitudinal axis or a radius.

In the configuration described above, at least two different enzymes can be used and catalyze the transformation of different analytes, with at least one enzyme in EZL2 756 providing hydrogen peroxide and the at least other enzyme in EZL1 755 not providing hydrogen peroxide, e.g., providing electron transfer to the WE surface corresponding directly or indirectly to a concentration of the analyte.

In one example, an inner layer of the at least two enzyme domains EZL1, EZL2 755, 756 comprises at least one immobilized enzyme in combination with at least one mediator that can facilitate lower bias voltage operation of the WE than without the mediator. In one example, for such direct electron transductions, a potential P1 is used. In one example, at least a portion of the inner layer EZL1 755 is more proximal to the WE surface and may have one or more intervening electrode domains and/or overlaying interference and/or bio-interfacing and/or drug releasing membranes, provided that the at least one mediator can facilitate low bias voltage operation with the WE surface. In another example, at least a portion of the inner layer EZL1 755 is directly adjacent the WE.

The second layer of at least dual enzyme domain (the outer layer EZL2 756) of FIG. 8B contains at least one enzyme that result in one or more catalysis reactions that eventually generate an amount of hydrogen peroxide that can electrochemically transduce a signal corresponding to the concentration of the analyte(s). In one example, the generated hydrogen peroxide diffuses through layer EZL2 756 and through the inner layer EZL1 755 to reach the WE surface and undergoes redox at a potential of P2, where P2≠P1. In this way electron transfer and electrolysis (redox) can be selectively controlled by controlling the potentials P1, P2 applied at the same WE surface. Any applied potential durations can be used for P1, P2, for example, equal/periodic durations, staggered durations, random durations, as well as various potentiometric sequences, cyclic voltammetry etc. In some examples, impedimetric sensing may be used. In one example, a phase shift (e.g., a time lag) may result from detecting two signals from two different working electrodes, each signal being generated by a different EZL (EZL1, EZL2, 755, 756) associated with each electrode. The two (or more) signals can be broken down into components to detect the individual signal and signal artifacts generated by each of EZL1 755 and EZL2 756 in response to the detection of two analytes. In some examples, each EZL detects a different analyte. In other examples, both EZLs detect the same analyte.

In another alternative exemplary configuration, as shown in FIGS. 8C-8D a multienzyme domain configuration as described above is provided for a continuous multi-analyte sensor device using a single WE with two or more active surfaces is provided. In one example, the multienzyme domain configurations discussed herein are formed on a planar substrate. In another example, the single WE is coaxial, e.g., configured as a wire, having two or more active surfaces positioned about the longitudinal axis of the wire. Additional wires can be used, for example, as a reference and/or counter electrode. In another example, the single WE is a conductive trace on a substrate, with two or more active surfaces positioned about the longitudinal axis of the trace. At least a portion of the two or more active surfaces are discontinuous, providing for at least two physically separated WE surfaces on the same WE wire or trace. (e.g., WE1, WE2). In one example, the first analyte detected by WE1 is glucose, and the second analyte detected by WE2 is lactate. In another example, the first analyte detected by WE1 is glucose, and the second analyte detected by WE2 is ketones.

Thus, FIGS. 8C-8D depict exemplary configurations of a continuous multi-analyte sensor construct in which EZL1 755, EZL2 756 and RL 752 (resistance domain) as described above, arranged, for example, by sequential dip coating techniques, over a single coaxial wire comprising spatially separated electrode surfaces WE1, WE2. One or more parameters, independently, of the enzyme domains, resistance domains, etc., can be controlled along the longitudinal axis of the WE, for example, thickness, length along the axis from the distal end of the wire, etc. In one example, at least a portion of the spatially separated electrode surfaces are of the same composition. In another example, at least a portion of the spatially separated electrode surfaces are of different composition. In FIGS. 8C-8D, WE1 represents a first working electrode surface configured to operate at P1, for example, and is electrically insulated from second working electrode surface WE2 that is configured to operate at P2, and RE represents a reference electrode RE electrically isolated from both WE1, WE2. One resistance domain is provided in the configuration of FIG. 8C that covers the reference electrode and WE1, WE2. An addition resistance domain is provided in the configuration of FIG. 8D that covers extends over essentially WE2 only. Additional electrodes, such as a counter electrode can be used. Such configurations (whether single wire or dual wire configurations) can also be used to measure the same analyte using two different techniques. Using different signal generating sequences as well as different RLs, the data collected from two different mode of measurements provides increase fidelity, improved performance and device longevity. A non-limiting example is a glucose oxidase (H2O2 producing) and glucose dehydrogenase (electrically coupled) configuration. Measurement of Glucose at two potentials and from two different electrodes provides more data points and accuracy. Such approaches may not be needed for glucose sensing, but the can be applied across the biomarker sensing spectrum of other analytes, alone or in combination with glucoses sensing, such as ketone sensing, ketone/lactate sensing, and ketone/glucose sensing.

In an alternative configuration of that depicted in FIGS. 8C-8D, two or more wire electrodes, which can be colinear, wrapped, or otherwise juxtaposed, are presented, where WE1 is separated from WE2, for example, from other elongated shaped electrode. Insulating layer electrically isolates WE1 from WE2. In this configuration, independent electrode potential can be applied to the corresponding electrode surfaces, where the independent electrode potential can be provided simultaneously, sequentially, or randomly to WE1, WE2. In one example, electrode potentials presented to the corresponding electrode surfaces WES1, WES2, are different. One or more additional electrodes can be present such as a reference electrode and/or a counter electrode. In one example, WES2 is positioned longitudinally distal from WES1 in an elongated arrangement. Using, for example, dip coating methods, WES1 and WES2 are coated with enzyme domain EZL1, while WES2 is coated with different enzyme domain EZL2. Based on the dipping parameters, or different thickness of enzyme domains, multi-layered enzyme domains, each layer independently comprising different loads and/or compositions of enzyme and/or cofactors, mediators can be employed. Likewise, one or more resistance domains (RL) can be applied, each can be of a different thickness along the longitudinal axis of the electrode, and over different electrodes and enzyme domains by controlling dip length and other parameters, for example. With reference to FIG. 8D, such an arrangement of RL's is depicted, where an additional RL 752′ is adjacent WES2 but substantially absent from WES1.

In one example of measuring two different analytes, the above configuration comprising enzyme domain EZL1 755 comprising one or more enzyme(s) and one or more mediators for at least one enzyme of EZL1 to provide for direct electron transfer to the WES1 and determining a concentration of at least a first analyte. In addition, enzyme domain EZL2 756 can comprise at least one enzyme that provides peroxide (e.g., hydrogen peroxide) or consumes oxygen during catalysis with its substrate. The peroxide or the oxygen produced in EZL2 756 migrates to WES2 and provides a detectable signal that corresponds directly or indirectly to a second analyte. For example, WES2 can be carbon, wired to glucose dehydrogenase to measure glucose, while WES1 can be platinum, that measures peroxided produced from lactate oxidase/lactate in EZL2 756. The combinations of electrode material and enzyme(s) as disclosed herein are examples and non-limiting.

In one example, the potentials of P1 and P2 can be separated by an amount of potential so that both signals (from direct electron transfer from EZL1 755 and from hydrogen peroxide redox at WE) can be separately activated and measured. In one example, the electronic module of the sensor can switch between two sensing potentials continuously in a continuous or semi-continuous periodic manner, for example a period (t1) at potential P1, and period (t2) at potential P2 with optionally a rest time with no applied potential. Signal extracted can then be analyzed to measure the concentration of the two different analytes. In another example, the electronic module of the sensor can undergo cyclic voltammetry, providing changes in current when swiping over potentials of P1 and P2 can be correlated to transduced signal coming from either direct electron transfer or electrolysis of hydrogen peroxide, respectably. In one example, the modality of sensing is non-limiting and can include different amperometry techniques, e.g., cyclic voltammetry. In one example, an alternative configuration is provided but hydrogen peroxide production in EZL2 is replaced by another suitable electrolysis compound that maintains the P2≠P1 relationship, such as oxygen, and at least one enzyme-substrate combination that provide the other electrolysis compound.

For example, a continuous multi-analyte sensor configuration, for choline and glucose, in which enzyme domains EZ1 755, EZ2 756 were associated with different WEs, e.g., platinum WE2, and gold WE1 was prepared. In this exemplary case, EZL1 755 contained glucose oxidase and a mediator coupled to WE1 to facilitate electron direct transfer upon catalysis of glucose, and EZL2 756 contained choline oxidase that will catalyze choline and generate hydrogen peroxide for electrolysis at WE2. The EZL's were coated with resistance domains; upon cure and readiness they underwent cyclic voltammetry in the presence of glucose and choline. A wired glucose oxidase enzyme to a gold electrode is capable of transducing signal at 0.2 volts, therefore, by analyzing the current changes at 0.2 volts, the concentration of glucose can be determined. The data also demonstrates that choline concentration is also inferentially detectable at the WE2 platinum electrode if the CV trace is analyzed at the voltage P2.

In one example, either electrode WE1 or WE2 can be, for example, a composite material, for example a gold electrode with platinum ink deposited on top, a carbon/platinum mix, and or traces of carbon on top of platinum, or porous carbon coating on a platinum surface. In one example, with the electrode surfaces containing two distinct materials, for example, carbon used for the wired enzyme and electron transfer, while platinum can be used for hydrogen peroxide redox and detection. As shown in FIG. 8E, an example of such composite electrode surfaces is shown, in which an extended platinum covered wire 757 is half coated with carbon 758, to facilitate multi sensing on two different surfaces of the same electrode. In one example WE2 can be grown on or extend from a portion of the surface or distal end of WE1, for example, by vapor deposition, sputtering, or electrolytic deposition and the like.

Additional examples include a composite electrode material that may be used to form one or both of WE1 and WE2. In one example, a platinum-carbon electrode WE1, comprising EZL1 with glucose dehydrogenase is wired to the carbon surface, and outer EZL2 comprising lactate oxidase generating hydrogen peroxide that is detectable by the platinum surface of the same WE1 electrode. Other examples of this configuration can include ketone sensing (beta-hydroxybutyrate dehydrogenase electrically coupled enzyme in EZL1 755) and glucose sensing (glucose oxidase in EZL2 756). Other membranes can be used in the aforementioned configuration, such as electrode, resistance, bio-interfacing, and drug releasing membranes. In other examples, one or both of the working electrodes (WE1, WE2) may be gold-carbon (Au—C), palladium-carbon (Pd—C), iridium-carbon (Ir—C), rhodium-carbon (Rh—C), or ruthenium-carbon (Ru—C). In some examples, the carbon in the working electrodes discussed herein may instead or additionally include graphene, graphene oxide, or other materials suitable for forming the working electrodes, such as commercially available carbon ink.

### Glycerol Sensor Configurations

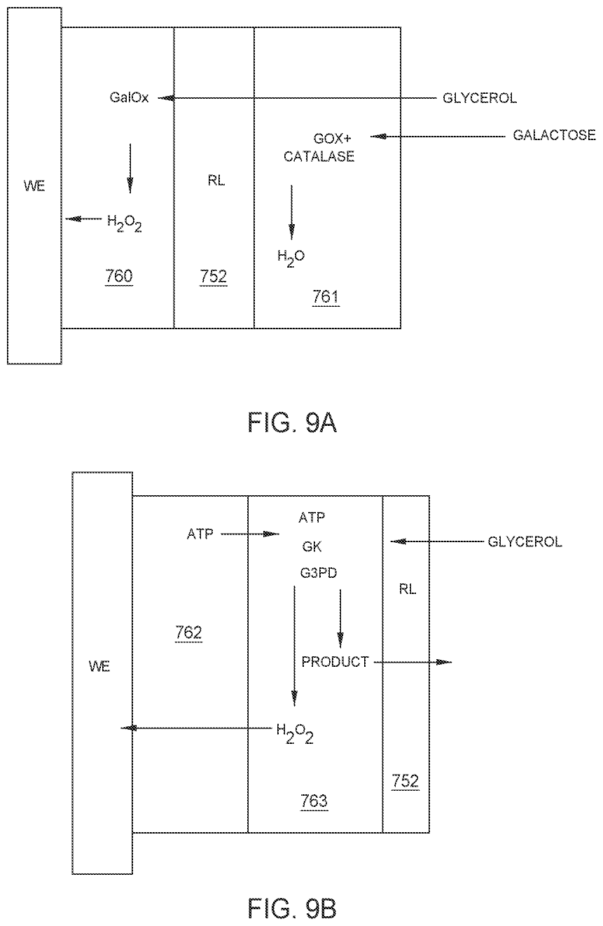

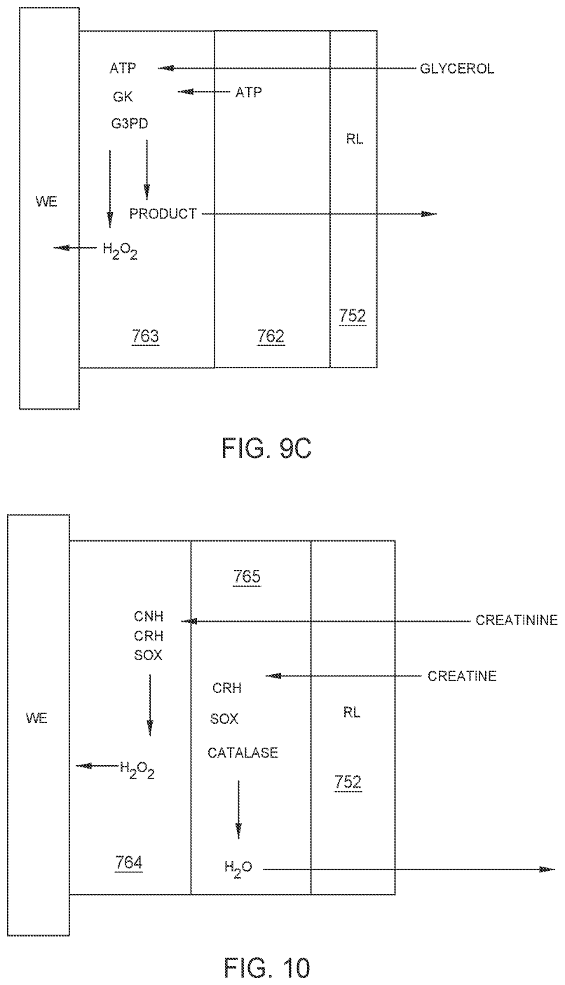

As shown in FIG. 9A, an exemplary continuous glycerol sensor configuration is depicted where a first enzyme domain EZL1 760 comprising galactose oxidase is positioned proximal to at least a portion of a WE surface. A second enzyme domain EZL2 761 comprising glucose oxidase and catalase is positioned more distal from the WE. As shown in FIG. 9A, one or more resistance domains (RL) 752 are positioned between EZL1 760 and EZL2 761. Additional RLs can be employed, for example, adjacent to EZL2 761. Modification of the one or more RL membranes to attenuate the flux of either analyte and increase glycerol to galactose sensitivity ratio is envisaged. The above glycerol sensing configuration provides for a glycerol sensor that can be combined with one or more additional sensor configurations as disclosed herein.

Glycerol can be catalyzed by the enzyme galactose oxidase (GalOx), however, GalOx has an activity ratio of 1%-5% towards glycerol. In one example, the activity of GalOx towards this secondary analyte glycerol can be utilized. The relative concentrations of glycerol in vivo are much higher that galactose (˜2 umol/l for galactose, and ˜100 umol/l for glycerol), which compliments the aforementioned configurations.

If the GalOx present in EZL1 760 membrane is not otherwise functionally limited, then the GalOx will catalyze most if not all of the glycerol that passes through the one or more RLs. The signal contribution from the glycerol present will be higher as compared to the signal contribution from galactose. In one example, the one or more RL's are chemically configured to provide a higher influx of glycerol or a lower influx of galactose.

In another example, a glycol sensor configuration is provided using multiple working electrodes WEs that provides for utilizing signal transduced from both WEs. Utilizing signal transduced from both WEs can provide increasing selectivity. In one example EZL1 760 and EZL2 761 comprise the same oxidase enzyme (e.g., galactose oxidase) with different ratios of enzyme loading, and/or a different immobilizing polymer and/or different number and layers of RL's over the WEs. Such configurations provide for measurement of the same target analyte with different sensitivities, resulting in a dual measurement. Using a mathematical algorithm to correct for noise and interference from a first signal, and inputting the first signal from one sensing electrode with a first analyte sensitivity ratio into the mathematical algorithm, allows for the decoupling of the second signal corresponding to the desired analyte contributions. Modification of the sensitivity ratio of the one or more EZL's to distinguish signals from the interfering species and the analyte(s) of interest can be provided by adjusting one or more of enzyme source, enzyme load in EZL's, chemical nature/diffusional characteristics of EZL's, chemical/diffusional characteristics of the at least one RL's, and combinations thereof.

As discussed herein, a secondary enzyme domain can be utilized to catalyze the non-target analyte(s), reducing their concentration and limiting diffusion towards the sensing electrode through adjacent membranes that contains the primary enzyme and necessary additives. In this example, the most distal enzyme domain, EZL2, 761 is configured to catalyze a non-target analyte that would otherwise react with EZL1, thus providing a potentially less accurate reading of the target analyte (glycerol) concentration. This secondary enzyme domain can act as a “selective diffusion exclusion membrane” by itself, or in some other configurations can be placed above or under a resistant layer (RL) 752. In this example, the target analyte is glycerol and GalOX is used to catalyze glycerol to form a measurable species (e.g., hydrogen peroxide).

In one example, a continuous glycerol sensor configuration is provided using at least glycerol oxidase, which provides hydrogen peroxide upon reaction and catalysis of glycerol. Thus, in one example, enzyme domain comprising glycerol oxidase can be positioned adjacent at least a portion of a WE surface and hydrogen peroxide is detected using amperometry. In another example, enzyme domain comprising glycerol oxidase is used for sensing oxygen level changes, for example, in a Clark type electrode setup. Alternatively, at least a portion of the WE surface can be coated with one more layers of electrically coupled polymers, such as a mediator system discussed below, to provide a coated WE capable of electron transfer from the enzyme at a lower potential. The coated WE can then operate at a different and lower voltage to measure oxygen and its correlation to glycerol concentration.

In another example, a glycerol sensor configuration is provided using glycerol-3-phosphate oxidase in the enzyme domain. In one example, ATP is used as the cofactor. Thus, as shown in FIGS. 9B and 9C, exemplary sensor configurations are depicted where in one example (FIG. 9B), one or more cofactors (e.g. ATP) 762 is proximal to at least a portion of an WE surface. One or more enzyme domains 763 comprising glycerol-3-phosphate oxidase (G3PD), lipase, and/or glycerol kinase (GK) and one or more regenerating enzymes capable of continuously regenerating the cofactor are contained in an enzyme domain are adjacent the cofactor, or more distal from the WE surface than the cofactor layer 762. Examples of regenerating enzymes that can be used to provide ATP regeneration include, but are not limited to, ATP synthase, pyruvate kinase, acetate kinase, and creatine kinase. The one or more regenerating enzymes can be included in one or more enzyme domains, or in a separate layer.

An alternative configuration is shown in FIG. 9C, where one or more enzyme domains 763 comprising G3PD, at least one cofactor and at least one regenerating enzyme, are positioned proximal to at least a portion of WE surface, with one or more cofactor reservoirs 762 adjacent to the enzyme domains comprising G3PD and more distal from the WE surface, and one or more RL's 752 are positioned adjacent the cofactor reservoir. In either of these configurations, an additional enzyme domain comprising lipase can be included to indirectly measure triglyceride, as the lipase will produce glycerol for detection by the aforementioned glycerol sensor configurations.

In another example, a glycerol sensor configuration is provided using dehydrogenase enzymes with cofactors and regenerating enzymes. In one example, cofactors that can be incorporated in the one or more enzyme domains include one or more of NAD(P)H, NADP+, and ATP. In one example, e.g., for use of NAD(P)H a regenerating enzyme can be NADH oxidase or diaphorase to convert NADH, the product of the dehydrogenase catalysis back to NAD(P)H. Similar methodologies can be used for creating other glycerol sensors, for example, glycerol dehydrogenase, combined with NADH oxidase or diaphorase can be configured to measure glycerol or oxygen.

In one example, mathematical modeling can be used to identify and remove interference signals, measuring very low analyte concentrations, signal error and noise reduction so as to improve and increase of multi-analyte sensor end of life. For example, with a two WE electrode configuration where WE1 is coated with a first EZL while WE2 is coated with two or more different EZL, optionally with one or more resistance domains (RL) a mathematical correction such interference can be corrected for, providing for increasing accuracy of the measurements.

Changes of enzyme load, immobilizing polymer and resistance domain characteristics over each analyte sensing region can result in different sensitive ratios between two or more target analyte and interfering species. If the signal are collected and analyzed using mathematical modeling, a more precise concentration of the target analytes can be calculated.

One example in which use of mathematical modeling can be helpful is with glycerol sensing, where galactose oxidase is sensitive towards both galactose and glycerol. The sensitivity ratio of galactose oxidase to glycerol is about is 1%-5% of its sensitivity to galactose. In such case, modification of the sensitivity ratio to the two analytes is possible by adjusting the one or more parameters, such as enzyme source, enzyme load, enzyme domain (EZL) diffusional characteristics, RL diffusional characteristics, and combinations thereof. If two WEs are operating in the sensor system, signal correction and analysis from both WEs using mathematical modeling provides high degree of fidelity and target analyte concentration measurement.

In the above configurations, the proximity to the WE of one or more of these enzyme immobilizing layers discussed herein can be different or reversed, for example if the most proximal to the WE enzyme domain provides hydrogen peroxide, this configuration can be used.

In some examples, the target analyte can be measured using one or multiple of enzyme working in concert. In one example, ATP can be immobilized in one or more EZL membranes, or can be added to an adjacent layer alone or in combination with a secondary cofactor, or can get regenerated/recycled for use in the same EZL or an adjacent third EZL. This configuration can further include a cofactor regenerator enzyme, e.g., alcohol dehydrogenase or NADH oxidase to regenerate NAD(P)H. Other examples of cofactor regenerator enzymes that can be used for ATP regeneration are ATP synthase, pyruvate kinase, acetate kinase, creatine kinase, and the like.

In one example, the aforementioned continuous glycerol sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations to provide a continuous multi-analyte sensor device as further described below. This continuous multi-analyte sensor device can further include continuous glucose monitoring capability. Other configurations can be used in the aforementioned continuous glycerol sensor configuration, such as electrode, resistance, bio-interfacing, and drug releasing membranes.

### Creatinine Sensor Configurations

In one example, continuous creatinine sensor configurations are provided, such configurations containing one or more enzymes and/or cofactors. Creatinine sensor configurations are examples of continuous analyte sensing systems that generate intermediate, interfering products, where these intermediates/interferents are also present in the biological fluids sampled. The present disclosure provides solutions to address these technical problems and provide for accurate, stable, and continuous creatinine monitoring alone or in combination with other continuous multi-analyte sensor configurations.

Creatinine sensors, when in use, are subject to changes of a number of physiologically present intermediate/interfering products, for example sarcosine and creatine, that can affect the correlation of the transduced signal with the creatinine concentration. The physiological concentration range of sarcosine, for example, is an order of magnitude lower that creatinine or creatine, so signal contribution from circulating sarcosine is typically minimal. However, changes in local physiological creatine concentration can affect the creatinine sensor signal. In one example, eliminating or reducing such signal contribution is provided.

Thus, in one example, eliminating or reducing creatine signal contribution of a creatinine sensor comprises using at least one enzyme that will consume the non-targeted interfering analyte, in this case, creatine. For example, two enzyme domains are used, positioned adjacent to each other. At least a portion of a first enzyme domain is positioned proximal to at least a portion of a WE surface, the first enzyme domain comprising one or more enzymes selected from creatinine amidohydrolase (CNH), creatine amidohydrolase (CRH), and sarcosine oxidase (SOX). A second enzyme domain, adjacent the first enzyme domain and more distal from the WE surface, comprises one or more enzymes using creatine as their substrate so as to eliminate or reduce creatine diffusion towards the WE. In one example, combinations of enzymes include CRH, SOX, creatine kinase, and catalase, where the enzyme ratios are tuned to provide ample number of units such that circulating creatine will at least partially be consumed by CRH providing sarcosine and urea, whereas the sarcosine produced will at least partially be consumed by SOX, providing an oxidized form of glycine (e.g. glycine aldehyde) which will at least be partially consumed by catalase. In an alternative configuration of the above, the urea produced by the CRH catalysis can at least partially be consumed by urease to provide ammonia, with the aqueous form (NH4+) being detected via an ion-selective electrode (e.g., nonactin ionophore). Such an alternative potentiometric sensing configuration may provide an alternative to amperometric peroxide detection (e.g., improved sensitivity, limits of detection, and lack of depletion of the reference electrode, alternate pathways/mechanisms). This dual-analyte-sensing example may include a creatinine-potassium sensor having potentiometric sensing at two different working electrodes. In this example, interference signals can be identified and corrected. In one alternative example, the aforementioned configuration can include multi-modal sensing architectures using a combination of amperometry and potentiometry to detect concentrations of peroxide and ammonium ion, measured using amperometry and potentiometry, respectively, and correlated to measure the concentration of the creatinine. In one example, the aforementioned configurations can further comprise one or more configurations (e.g., without enzyme) separating the two enzyme domains to provide complementary or assisting diffusional separations and barriers.

In yet another example, a method to isolate the signal and measure essentially only creatinine is to use a second WE that measures the interfering species (e.g., creatine) and then correct for the signal using mathematical modeling. Thus, for example, signal from the WE interacting with creatine is used as a reference signal. Signal from another WE interacting with creatinine is from corrected for signal from the WE interacting with creatine to selectively determine creatinine concentration.

In yet another example, sensing creatinine is provided by measuring oxygen level changes electrochemically, for example in a Clark type electrode setup, or using one or more electrodes coated with layers of different polymers such as NAFION™ and correlating changes of potential based on oxygen changes, which will indirectly correlate with the concentrations of creatinine.

In yet another example, sensing creatinine is provided by using sarcosine oxidase wired to at least one WE using one or more electrically coupled mediators. In this approach, concentration of creatinine will indirectly correlate with the electron transfer generated signal collected from the WE.

For the aforementioned creatinine sensor configurations based on hydrogen peroxide and/or oxygen measurements the one or more enzymes can be in a single enzyme domain, or the one or more enzymes, independently, can be in one or more enzyme domains, or any other combination thereof, in which in each layer at least one enzyme is present. For the aforementioned creatinine sensor configurations based on use of an electrically coupled sarcosine oxidase containing layer, the layer positioned adjacent to the electrode and is electrically coupled to at least a portion of the electrode surface using mediators.

In another example, the aforementioned creatinine sensor configurations can be sensed using potentiometry by using urease enzyme (UR) that creates ammonium from urea, the urea created by CRH from creatine, the creatine being formed from the interaction of creatinine with CNH. Thus, ammonium can be measured by the above configuration and correlated with the creatinine concentration. Alternatively, creatine amidohydrolase (CI) or creatinine deiminase can be used to create ammonia gas, which under physiological conditions of a transcutaneous sensor, would provide ammonium ion for signal transduction.

In yet another example, sensing creatinine is provided by using one or more enzymes and one or more cofactors. Some non-limiting examples of such configurations include creatinine deaminase (CD) providing ammonium from creatinine, glutamate dehydrogenase (GLDH) providing peroxide from the ammonium, where hydrogen peroxide correlates with levels of present creatinine. The above configuration can further include a third enzyme glutamate oxidase (GLOD) to further break down glutamate formed from the GDLH and create additional hydrogen peroxide. Such combinations of enzymes, independently, can be in one or more enzyme domains, or any other combination thereof, in which in each domain or layer, at least one enzyme is present.

In yet another example, sensing creatinine is provided by the combination of creatinine amidohydrolase (CNH), creatine kinase (CK) and pyruvate kinase (PK), where pyruvate, created by PK can be detected by one or more of either lactate dehydrogenase (LDH) or pyruvate oxidase (POX) enzymes configured independently, where one or more of the aforementioned enzyme are present in one layer, or, in which in each of a plurality of layers comprises at least one enzyme, any other combination thereof.

In such sensor configurations where one or more cofactors and/or regenerating enzymes for the cofactors are used, providing excess amounts of one or more of NADH, NAD(P)H and ATP in any of the one or more configurations can be employed, and one or more diffusion resistance domains can be introduced to limit or prevent flux of the cofactors from their respective membrane(s). Other configurations can be used in the aforementioned configurations, such as electrode, resistance, bio-interfacing, and drug releasing membranes.

In yet another example, creatinine detection is provided by using creatinine deiminase in one or more enzyme domains and providing ammonium to the enzyme domain(s) via catalysis of creatinine. Ammonium ion can then be detected potentiometrically or by using composite electrodes that undergo redox when exposed to ammonium ion, for example NAFION™/polyaniline composite electrodes, in which polyaniline undergoes redox in the presence of ammonium at the electrode under potential. Ammonium concentration can then be correlated to creatinine concentration.

FIG. 10 depicts an exemplary continuous sensor configuration for creatinine. In the example of FIG. 10, the sensor includes a first enzyme domain 764 comprising CNH, CRH, and SOX are adjacent a working electrode WE, e.g., platinum. A second enzyme domain 765 is positioned adjacent the first enzyme domain and is more distal from the WE. One or more resistance domains (RL) 752 can be positioned adjacent the second enzyme domain or between the first and second layers. Creatinine is diffusible through the RL and the second enzyme domain to the first enzyme domain where it is converted to peroxide and transduces a signal corresponding to its concentration. Creatine is diffusible through the RL and is converted in the second enzyme domain to sarcosine and urea, the sarcosine being consumed by the sarcosine oxidase and the peroxide generated is consumed by the catalase, thus preventing transduction of the creatine signal.

For example, variations of the above configuration are possible for continuous monitoring of creatinine alone or in combination with one or more other analytes. Thus, one alternative approach to sensing creatinine could be sensing oxygen level changes electrochemically, for example in a Clark-type electrode setup. In one example, the WE can be coated with layers of different polymers, such as NAFION™ and based on changes of potential oxygen changes, the concentrations of creatinine can be correlated. In yet another example, one or more enzyme most proximal to the WE, i.e., sarcosine oxidase, can be “wired” to the electrode using one or more mediators. Each of the different enzymes in the above configurations can be distributed inside a polymer matrix or domain to provide one enzyme domain. In another example, one or more of the different enzymes discussed herein can be formed as the enzyme domain and can be formed layer by layer, in which each layer has at least one enzyme present. In an example of a “wired” enzyme configuration with a multilayered membrane, the wired enzyme domain would be most proximal to the electrode. One or more interferent layers can be deposited among the multilayer enzyme configuration so as to block of non-targeted analytes from reaching electrodes.

In one example, the aforementioned continuous creatinine sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations, glycerol sensor configurations to provide a continuous multi-analyte sensor device as further described below. This continuous multi-analyte sensor device can further include continuous glucose monitoring capability.

### Lactose Sensor Configurations

In one example, a continuous lactose sensor configuration, alone or in combination with another analyte sensing configuration comprising one or more enzymes and/or cofactors is provided. In a general sense, a lactose sensing configuration using at least one enzyme domain comprising lactase enzyme is used for producing glucose and galactose from the lactose. The produced glucose or galactose is then enzymatically converted to a peroxide for signal transduction at an electrode. Thus, in one example, at least one enzyme domain EZL1 comprising lactase is positioned proximal to at least a portion of a WE surface capable of electrolysis of hydrogen peroxide. In one example, glucose oxidase enzyme (GOX) is included in EZL1, with one or more cofactors or electrically coupled mediators. In another example, galactose oxidase enzyme (GalOx) is included in EZL1, optionally with one or more cofactors or mediators. In one example, glucose oxidase enzyme and galactose oxidase are both included in EZL1. In one example, glucose oxidase enzyme and galactose oxidase are both included in EZL1, optionally with one or more cofactors or electrically coupled mediators.

One or more additional EZL's (e.g. EZL2) can be positioned adjacent the EZL1, where at least a portion of EZL2 is more distal from at least a portion of WE than EZL1. In one example, one or more layers can be positioned in between EZL1 and EZL2, such layers can comprise enzyme, cofactor or mediator or be essentially devoid of one or more of enzymes, cofactors or mediators. In one example, the one or more layers positioned in between EZL1 and EZL2 is essentially devoid of enzyme, e.g., no purposefully added enzyme. In one example one or layers can be positioned adjacent EZL2, being more distal from at least a portion of EZL1 than EZL2, and comprise one or more of the enzymes present in either EZL1 or EZL2.

In one example of the aforementioned lactose sensor configurations, the peroxide generating enzyme can be electrically coupled to the electrode using coupling mediators. The transduced peroxide signals from the aforementioned lactose sensor configurations can be correlated with the level of lactose present.

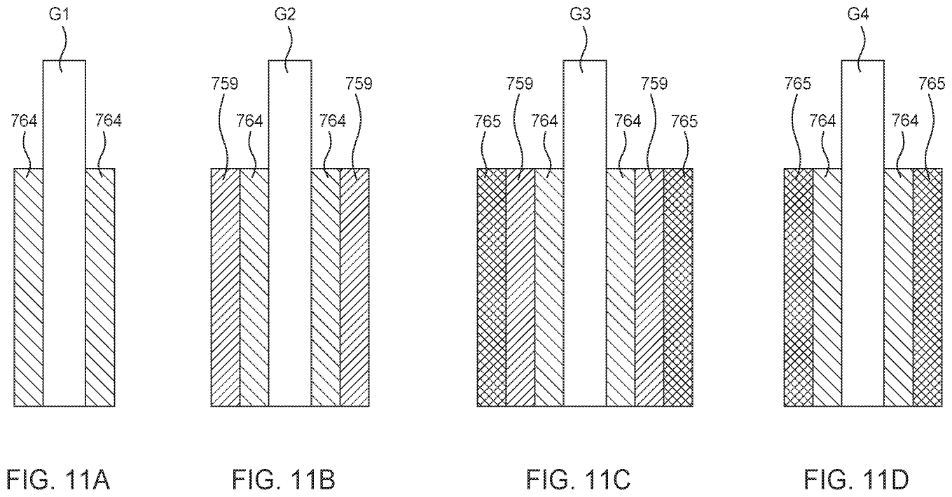

FIG. 11A-11D depict alternative continuous lactose sensor configurations. Thus, in an enzyme domain EZL1 764 most proximal to WE (G1), comprising GalOx and lactase, provides a lactose sensor that is sensitive to galactose and lactose concentration changes and is essentially non-transducing of glucose concentration. As shown in FIGS. 11B-11D, additional layers, including non-enzyme containing layers 759, and an enzyme domain 765 (e.g., a lactase enzyme containing layer), and optionally, electrode, resistance, bio-interfacing, and drug releasing membranes. (not shown) are used. Since changes in physiological galactose concentration are minimal, the transduced signal would essentially be from physiological lactose fluctuations.

In one example, the aforementioned continuous lactose sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations, glycerol sensor configurations, creatinine sensor configurations to provide a continuous multi-analyte sensor device as further described below. This continuous multi-analyte sensor device can further include continuous glucose monitoring capability. Other membranes can be used in the aforementioned sensor configuration, such as electrode, resistance, bio-interfacing, and drug releasing membranes.

### Urea Sensor Configurations

Similar approach as described above can also be used to create a continuous urea sensor. For example urease (UR), which can break down the urea and to provide ammonium can be used in an enzyme domain configuration. Ammonium can be detected with potentiometry or by using a composite electrodes, e.g., electrodes that undergo redox when exposed to ammonium. Example electrodes for ammonium signal transduction include, but are not limited to, NAFION™/polyaniline composite electrodes, in which polyaniline undergoes redox in the presence of ammonium at an applied potential, with essentially direct correlation of signal to the level of ammonium present in the surrounding. This method can also be used to measure other analytes such as glutamate using the enzyme glutaminase (GLUS).

In one example, the aforementioned continuous uric acid sensor configurations can be combined with any one of the aforementioned continuous alcohol sensor configurations and/or continuous uric acid sensor configurations and/or continuous cholesterol sensor configurations and/or continuous bilirubin/ascorbic acid sensor configurations and/or continuous ketone sensor configurations and/or continuous choline sensor configurations and/or continuous glycerol sensor configurations and/or continuous creatinine sensor configurations and/or continuous lactose sensor configurations to provide a continuous multi-analyte sensor device as further described below. This continuous multi-analyte sensor device can further include continuous glucose monitoring capability. Other membranes can be used in the aforementioned uric acid sensor configuration, such as electrode, resistance, bio-interfacing, and drug releasing membranes.

In certain embodiments, continuous analyte monitoring system 104 may be a potassium sensor, as discussed in reference to FIG. 1. FIGS. 7-14 describe an example sensor device used to measure an electrophysiological signal and/or concentration of a target analyte (e.g., potassium), according to certain embodiments of the present disclosure.

The term “ion” as used herein is a broad term, and is to be given its ordinary and customary meaning to a person of ordinary skill in the art (and is not to be limited to a special or customized meaning), and refers without limitation to an atom or molecule with a net electric charge due to the loss or gain of one or more electrons. Ions in a biological fluid may be referred to as “electrolytes.” Nonlimiting examples of ions in biological fluids include sodium (Na⁺), potassium (K⁺), magnesium (Mg²⁺), calcium (Ca²⁺), hydrogen (H⁺), lithium (Li⁺), chloride (Cl⁻), sulfide (S²⁻), sulfite (SO₃²⁻), sulfate (SO₄²⁻), phosphate (PO₄³⁻), and ammonium (NH₄⁺). An ion is an example of an analyte.

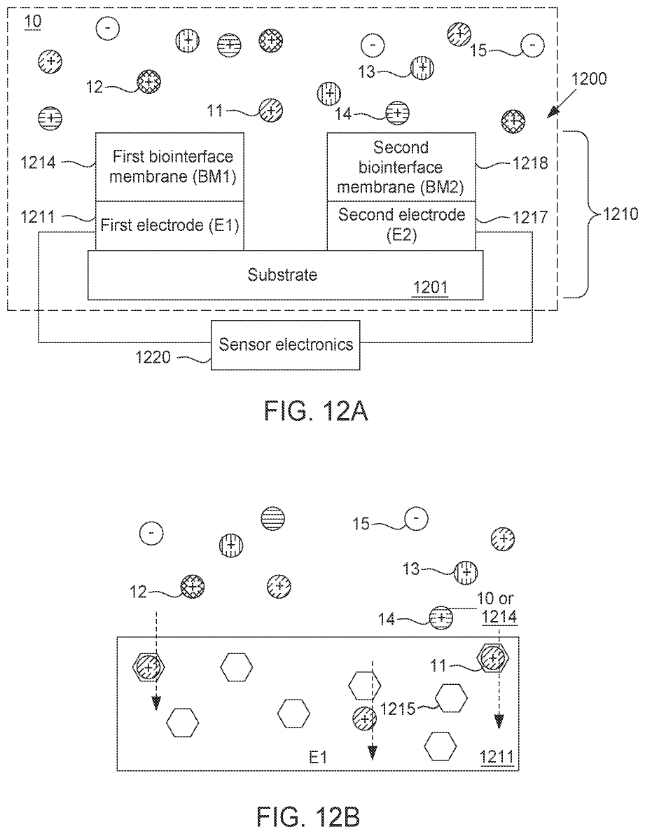

FIG. 12A schematically illustrates an example configuration and component of a device 1200 for measuring an electrophysiological signal and/or concentration of a target analyte such as a target ion 11 in a biological fluid 10 in vivo. Turning first to FIG. 12, device 1200 includes indwelling sensor 1210 and sensor electronics 1220. Sensor 1210 includes substrate 1201, first electrode (E1) 1211 disposed on the substrate, and a second electrode (E2) 1217 disposed on the substrate. First electrode 1211 may be referred to as a working electrode (WE), while second electrode 1217 may be referred to as a reference electrode (RE). The sensor electronics 1220 may be configured to generate a signal corresponding to an electromotive force (EMF). In some examples, the EMF is at least partially based on a potential difference that is generated between the first electrode 1211 and the second electrode 1217 responsive to biological fluid 10 conducting the electrophysiological signal to first electrode 1211. Sensor electronics 1220 may be configured to use the signal to generate an output corresponding to a measurement of the signal. In various examples, the EMF is at least partially based on a potential difference between (i) either the first electrode 1211 or the second electrode 1217 and (ii) another electrode which is spaced apart from the first electrode or second electrode.

Additionally, or alternatively, in some examples, device 1200 may include an ionophore, such as ionophore 1215 as shown in FIG. 12B, disposed on the substrate 1201 and configured to selectively transport the target ion 11 to or within the first electrode 1211. The EMF may be at least partially based on a potential difference may be generated between the first electrode 1211 and the second electrode 1217 responsive to the ionophore transporting the target ion to or through the first electrode 1211. The sensor electronics 1220 (and/or an external device that receives the signal via a suitable wired or wireless connection) may be configured to use the signal to generate an output corresponding to a measurement of the concentration of the target ion in the biological fluid. Further details regarding the configuration and use of sensor electronics 1220 are provided further below.

Optionally, the first electrode 1211 may be used to measure an electrophysiological signal in addition to ion concentration. In other examples, such as when device 1200 is configured to detect an electrophysiological signal but not an ion concentration, first electrode 1211 need not include an ionophore, such as ionophore 1215 as shown in FIG. 12B. In other examples, the first electrode 1211 may include an ionophore that is inactive such that it does not interfere with the measurement of the electrophysiological signal.

In a manner such as illustrated in FIG. 12A, biological fluid 10 may include a plurality of ions 11, 12, 13, 14, and 15. Device 1200 may be configured to measure the concentration of ion 11, and accordingly such ion may be referred to as a “target” ion. Target ion 11 may be any suitable ion, and in nonlimiting examples is selected from the group consisting of sodium (Na⁺), potassium (K⁺), magnesium (Mg²⁺), calcium (Ca²⁺), hydrogen (H⁺), lithium (Li⁺), chloride (Cl⁻), sulfite (SO₃²⁻), sulfate (SO₄²⁻), phosphate (PO₄³⁻), and ammonium (NH₄⁺). Ions 12, 13, 14, and 15 may be others of the group consisting of sodium (Na⁺), potassium (K⁺), magnesium (Mg²⁺), calcium (Ca²⁺), hydrogen (H⁺), lithium (Li⁺), chloride (Cl⁻), sulfide (S²⁻), sulfite (SO₃²⁻), sulfate (SO₄²⁻), phosphate (PO₄³⁻), and ammonium (NH₄⁺). Ions 12, 13, 14, and 15 may be considered interferents to the measurement of target ion 11 because they have the potential interfere with the measurement of target ion 11 by sensor to produce a signal that does not accurately represent the concentration of target ion 11. Ionophore, such as ionophore 1215 as shown in FIG. 12B, may be selected so as to selectively transport target ion 11 to or within first electrode 1211 and to inhibit, fully, partially and/or substantially, the transport of one or more of ions 12, 13, 14, or 15 to or within first electrode 1211. For example, as illustrated in FIG. 12B, ionophore 1215 may selectively transport, or selectively bind, target ions 11 from biological fluid 10 or from biointerface membrane 1214 (if provided, e.g., as described below) to and within first electrode 1211, while ions 12, 13, 14, and 15 may substantially remain within biological fluid 10 or biointerface membrane 1214. Accordingly, contributions to the potential difference between first electrode 1211 and second electrode 1217 responsive to the transport of ions to or within first electrode 1211 substantially may be primarily caused by target ion 11 instead of by one or more of ions 12, 13, 14, or 15.

A wide variety of ionophores 1215 may be used to selectively transport corresponding ions in a manner such as described with reference to FIGS. 12A-12B. For example, where the target ion 11 is hydrogen (via peroxide), the ionophore 1215 may be tridodecylamine, 4-nonadecylpyridine, N,N-dioctadecylmethylamine, octadecyl isonicotinate, calix[4]-aza-crown. Or, for example, where the target ion 11 is lithium, the ionophore 1215 may be ETH 149, N,N,N′,N′,N″,N″-hexacyclohexyl-4,4′,4″-propylidynetris(3-oxabutyramide), or 6,6-Dibenzyl-1,4,8-11-tetraoxacyclotetradecane. Or, for example, where the target ion 11 is sulfite, the ionophore 1215 may be octadecyl 4-formylbenzoate. Or, for example, where the target ion 11 is sulfate, the ionophore 1215 may be 1,3-[bis(3-phenylthioureidomethyl)]benzene or zinc phthalocyanine. Or, for example, where the target ion 11 is phosphate, the ionophore 1215 may be 9-decyl-1,4,7-triazacyclodecane-8,10-dione. Or, for example, where the target ion 11 is sodium, the ionophore 1215 may be 4-tert-butylcalix[4]arene-tetraacetic acid tetraethyl ester (sodium ionophore X) or calix[4]arene-25,26,27,28-tetrol (calix[4]arene). Or, for example, where the target ion 11 is potassium, the ionophore 1215 may be potassium ionophore II (BB15C5) or valinomycin. Or, for example, where the target ion 11 is magnesium, the ionophore 1215 may be 4,5-bis(benzo ylthio)-1,3-dithiole-2-thione (Bz2dmit) or 1,3,5-Tris[10-(1-adamantyl)-7,9-dioxo-6,10-diazaundecyl]benzene (magnesium ionophore VI). Or, for example, where the target ion 11 is calcium, the ionophore 1215 may be calcium ionophore I (ETH 1001) or calcium ionophore II (ETH129). Or, for example, where the target ion 11 is chloride, the ionophore 1215 may be tridodecylmethylammonium chloride (TDMAC). Or, for example, where the target ion 11 is ammonium, the ionophore 1215 may be nonactin.

In the nonlimiting example illustrated in FIG. 12A, ionophore 1215 may be provided within first electrode 1211, and in such example the first electrode may be referred to as an ion-selective electrode (ISE), since the ionophore 1215 selectively transports the target ion 11. In some examples, first electrode 1211 may include a conductive polymer optionally having ionophore 1215 therein. Illustratively, the conductive polymer may be present in an amount of about 90 to about 99.5 weight percent in the first electrode 1211. The ionophore 1215 may be present in an amount of about 0.5 to about 10 weight percent in the first electrode. In some examples, the conductive polymer may be selected from the group consisting of: poly(3,4-ethylenedioxythiophene) (PEDOT), poly(3,4-ethylenedioxythiophene) polystyrene sulfonate (PEDOT:PSS), polyaniline (PANI), poly(pyrrole) (PPy), or poly(3-octylthiophene) (POT).

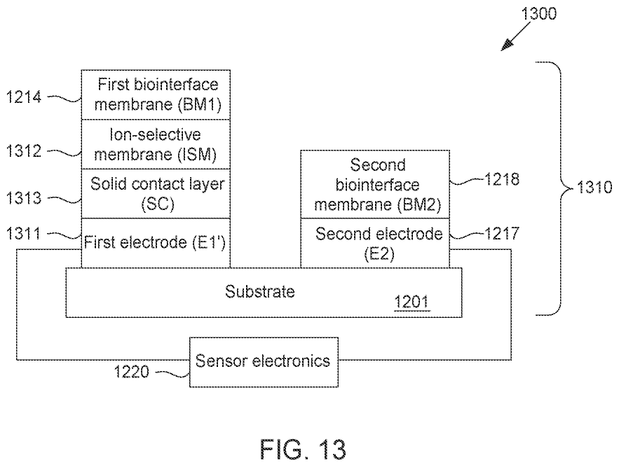

While conductive polymers (such as listed above) suitably may be used in a first electrode 1211 that excludes ionophore 1215, other materials alternatively may be used, some nonlimiting examples of which are described below with reference to FIG. 13. Optionally, ionophore 1215 may be provided in a membrane which is disposed on a first electrode 1211 (which electrode may exclude ionophore 1215), e.g., such as will be described below with reference to FIG. 13.

First electrode 1211 may be configured in such a manner as to enhance its biocompatibility. For example, first electrode 1211 may substantially exclude any plasticizer, which otherwise may leach into the biological fluid 10, potentially causing toxicity and/or a degradation in device performance. As used herein, the “substantial” exclusion of materials such as plasticizers is intended to mean that the first electrode 1211 or other aspects discussed herein do not contain detectable quantities of the “substantially” excluded material. In some examples, the first electrode 1211 may consist essentially of the conductive polymer, optionally in addition to the ionophore 1215. In some examples, the first electrode 1211 may consist essentially of the conductive polymer, the ionophore 1215, and an additive with ion exchanger capability. Such an additive may act as an ion exchanger. In one example, the additive contributes to the ion selectivity. In another example, the additive may not provide ion selectivity. For example, the additive may help to provide a substantially even concentration of the ion in the membrane. Additionally, or alternatively, the additive may help any change in ion concentration in the biofluid to cause an ion exchange within the membrane that may induce a non-selective potential difference. Additionally, or alternatively, the ionophore and the ion exchanger may form a complex which improves the ionophore's selectivity towards the target ion as compared to the selectivity of the ionophore alone.

Optionally, the additive may include a lipophilic salt. In nonlimiting examples, the lipophilic salt is selected from the group consisting of sodium tetrakis[3,5-bis(trifluoromethyl)phenyl]borate (NaTPFB), sodium tetraphenylborate (NaTPB), potassium tetrakis [3,5-bis(trifluoromethyl)phenyl]borate (KTFPB), and potassium tetrakis(4-chlorophenyl)borate (KTClPB). The additive may be present in an amount of about 0.01 to about 1 weight percent in the first electrode, or other suitable amount.

Other materials within sensor 1210 may be selected. For example, substrate 1201 may include a material selected from the group consisting of: metal, glass, transparent conductive oxide, semiconductor, dielectric, ceramic, and polymer (such as biopolymer or synthetic polymer). In some examples, second electrode 1317 may include a metal, a metal alloy, a transition metal oxide, a transparent conductive oxide, a carbon material, a doped semiconductor, a binary semiconductor, a ternary semiconductor, or a conductive polymer. The binary semiconductor may include any two elements suitable for use in a semiconductor. The ternary semiconductor may include two or more binary semiconductors. In examples where a metal or a metal alloy is used, the metal or metals used can be selected from the group consisting of: gold, platinum, silver, iridium, rhodium, ruthenium, nickel, chromium, and titanium. The metal optionally may be oxidized or optionally may be in the form of a metal salt. A nonlimiting example of an oxidized metal which may be used in second electrode 1217 is iridium oxide. The carbon material may be selected from the group consisting of: carbon paste, graphene oxide, carbon nanotubes, C60, porous carbon nanomaterial, mesoporous carbon, glassy carbon, hybrid carbon nanomaterial, graphite, and doped diamond. The doped semiconductor may be selected from the group consisting of: silicon, germanium, silicon-germanium, zinc oxide, gallium arsenide, indium phosphide, gallium nitride, cadmium telluride, indium gallium arsenide, and aluminum arsenide. The transition metal oxide may be selected from the group of: titanium dioxide (TiO₂), iridium dioxide (IrO₂), platinum dioxide (PtO₂), zinc oxide (ZnO), copper oxide (CuO), cerium dioxide (CeO₂), ruthenium(IV) oxide (RuO₂), tantalum pentoxide (Ta₂O₅), titanium dioxide (TiO₂), molybdenum dioxide (MoO₂), and manganese dioxide (MnO₂). The metal alloy may be selected from the group consisting of: platinum-iridium (Pt—Ir), platinum-silver (Pt—Ag), platinum-gold (Pt—Au), gold-iridium (Au—Ir), gold-copper (Au—Cu), gold-silver (Au—Ag), and cobalt-iron (Co—Fe).

The conductive polymer that may be used for the sensor 2010 may be selected from the group consisting of: poly(3,4-ethylenedioxythiophene) (PEDOT), poly(3,4-ethylenedioxythiophene) polystyrene sulfonate (PEDOT:PSS), polyaniline (PANI), poly(pyrrole) (PPy), or poly(3-octylthiophene) (POT). That is, first electrode 1311 and second electrode 1317 optionally may be formed of the same material as one another, or may be formed using different materials than one another. In the nonlimiting example illustrated in FIG. 12A, first electrode 1211 and second electrode 1217 may be disposed directly on substrate 1201, or alternatively may be disposed on substrate 1201 via one or more intervening layers (not illustrated).

The biocompatibility of sensor 1210 optionally may be further enhanced by providing a biointerface membrane over one or more component(s) of sensor 1210. For example, in the nonlimiting configuration illustrated in FIG. 12A, a first biointerface membrane (BM1) 1214 may be disposed on the ionophore 1215 and the first electrode 1211. In another example, the first biointerface membrane (BM1) 1214 may be disposed on the ionophore 1215 and the first electrode 1211, and a second biointerface membrane (BM2) 1218 may be disposed on the second electrode 1217. Although FIG. 12A may suggest that the biointerface membrane(s) have a rectangular shape for simplicity of illustration, it should be apparent that the membrane(s) may conform to the shape of any underlying layers. In some examples, the biointerface membrane(s) may be configured to inhibit biofouling of the ionophore 1215, the first electrode 1211, and/or the second electrode 1217. Nonlimiting examples of materials which may be included in the biointerface membrane(s) include hard segments and/or soft segments. Examples of hard and soft segments used for the biointerface membrane 1214/1214′/1218 or other biointerface membranes as discussed herein include aromatic polyurethane hard segments with Si groups, aliphatic hard segments, polycarbonate soft segments or any combination thereof. In other examples of biointerface membrane(s) such as 1214/1214′/1218 or other biointerface membranes discussed herein, PVP may not be included. In this example where no PVP is included, the biointerface membrane (1218, 1214, 1214′, or other biointerface membranes as discussed herein) may include polyurethane and PDMS. In some examples, which may be combined with other examples herein, the biointerface membranes discussed herein may include one or more zwitterionic compounds.

Whereas ionophore 1215 is included within first electrode 1211 in the example described with reference to FIG. 12A, in the example illustrated in FIG. 13 first electrode 1311 does not include ionophore 1215 (and thus may be referred to as E1′ rather than E1). Instead, ionophore 1215 may be within an ion-selective membrane (ISM) 1312 disposed on the first electrode 1311. Ionophores 1215 may selectively transport target ion 11 to first electrode 1311 in a manner similar to that described with reference to FIGS. 12A-12B, and such transport may cause a potential difference between the first electrode 1311 and second electrode 1217 based upon which sensor electronics 1220 may generate an output corresponding to a measurement of the concentration of target ion 11 in biological fluid 10. It will be appreciated that in examples in which device 1200 is used to measure an electrophysiological signal and is not used to measure an ion concentration, ISM 1312 may be omitted.

In a manner similar to that described with reference to first electrode 1211, ion-selective membrane 1312 substantially may exclude any plasticizer. In some examples, ion-selective membrane 1312 may consist essentially of a biocompatible polymer and ionophore 1215 configured to selectively bind the target ion. Alternatively, in some examples, the ion-selective membrane 1312 may consist essentially of a biocompatible polymer, an ionophore 1215 configured to selectively bind the target ion 11, and an additive with ion exchanger capability, such as a lipophilic salt. Nonlimiting examples of lipophilic salts, and nonlimiting amounts of additives, biocompatible polymers, and ionophores are provided above with reference to FIGS. 12A-12B. Whereas first electrode 1211 includes a conductive polymer so as to be able to provide ionophore 1215 therein while retaining the electrical conductivity of an electrode, additional types of materials may be used in ion-selective membrane 1312 because the ion-selective membrane 1312 need not be used as an electrode. For example, the biocompatible polymer of the ion-selective membrane 1312 may include a hydrophobic polymer. Illustratively, the hydrophobic polymer may be selected from the group consisting of silicone, fluorosilicone (FS), polyurethane, polyurethaneurea, polyurea. In one example, the biocompatible polymer of the ISM 1412 (or other ion-selective membranes or other membranes discussed here) may include one or more block copolymers, which may be segmented block copolymers. In one example, the hydrophobic polymer may be a segmented block copolymer comprising polyurethane and/or polyurea segments, and/or polyester segments, and one or more of polycarbonate, polydimethylsiloxane (PDMS), methylene diphenyl diisocyanate (MDI), polysulfone (PSF), methyl methacrylate (MMA), poly(ε-caprolactone) (PCL), and 1,4-butanediol (BD). In other examples, the hydrophobic polymer may alternately or additionally include poly(vinyl chloride) (PVC), fluoropolymer, polyacrylate, and/or polymethacrylate.

In one example, the biocompatible polymer may include a hydrophilic block copolymer instead of or in addition to one or more hydrophobic copolymers. Illustratively, the hydrophilic block copolymer may include one or more hydrophilic blocks selected from the group consisting of polyethylene glycol (PEG) and cellulosic polymers. Additionally, or alternatively, the block copolymer may include one or more hydrophobic blocks selected from the group consisting of polydimethylsiloxane (PDMS) polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polybutylene terephthalate (PBT), polymethylmethacrylate (PMMA), polyether ether ketone (PEEK), polyurethanes, poly(propylene oxide) and copolymers and blends thereof. In one example, the ion-selective membrane 2112 does not contain PVP, or other plasticizers.

In one example, the biocompatible polymer of the ion-selective membrane 1312 includes from about 0.1 wt. % silicone to about 80 wt. % silicone. In one example, the ion-selective membrane 1312, or other ion-selective membranes discussed herein, includes from about 5 wt. % silicone to about 25 wt. % silicone. In yet another example, the ion-selective membrane 1312, or other ion-selective membranes discussed herein, includes from about 35 wt. % silicone to about 65 wt. % silicone. In yet another example, the ion-selective membrane 1312, or other ion-selective membranes discussed herein, includes from about 30 wt. % silicone to about 50 wt. % silicone.

In certain examples, the ISM 1312 or other ISMs discussed herein may include one or more block copolymers or segmented block copolymers. The segmented block copolymer may include hard segments and soft segments. In this example, the hard segments may include aromatic or aliphatic diisocyanates are used to prepare hard segments of segmented block copolymer. In one example, the aliphatic or aromatic diisocyanate used to provide hard segment of polymer includes one or more of norbornane diisocyanate (NBDI), isophorone diisocyanate (IPDI), tolylene diisocyanate (TDI), 1,3-phenylene diisocyanate (MPDI), trans-1,3-bis(isocyanatomethyl) cyclohexane (1,3-H6XDI), bicyclohexylmethane-4,4′-diisocyanate (HMDI), 4,4′-diphenylmethane diisocyanate (MDI), trans-1,4-bis(isocyanatomethyl)cyclohexane (1,4-H6XDI), 1,4-cyclohexyl diisocyanate (CHDI), 1,4-phenylene diisocyanate (PPDI), 3,3′-Dimethyl-4,4′-biphenyldiisocyanate (TODI), 1,6-hexamethylene diisocyanate (HDI), or combinations thereof. In one example, the hard segments may be from about 5 wt. % to about 90 wt. % of the segmented block copolymer of the ISM 1312. In another example, the hard segments may be from about 15 wt. % to about 75 wt. %. In yet another example, the hard segments may be from about 25 wt. % to about 55 wt. %. It will be appreciated that ion-selective membrane 1312 and first electrode 1211 may be prepared in any suitable manner. Illustratively, the polymer, ionophore 1215, and any additive may be dispersed in appropriate amounts in a suitable organic solvent (e.g., tetrahydrofuran, isopropyl alcohol, acetone, or methyl ethyl ketone). The mixture may be coated onto substrate 1201 (or onto a layer thereon) using any suitable technique, such as dipping and drying, spray-coating, inkjet printing, aerosol jet dispensing, slot-coating, electrodeposition, electrospraying, electrospinning, chemical vapor deposition, plasma polymerization, physical vapor deposition, spin-coating, or the like. The organic solvent may be removed so as to form a solid material corresponding to ion-selective membrane 1312 or first electrode 1211. Other layers in device 1200 or device 1300, such as electrodes, solid contact layers, and/or biological membranes, may be formed using techniques described elsewhere herein or otherwise known in the art.

Whereas first electrode 1211 includes a conductive polymer so as to be able to provide ionophore 1215 therein while retaining the electrical conductivity of an electrode, additional types of materials may be used in first electrode 1311 because an ionophore need not be provided therein. Nonlimiting example materials for use in first electrode 1311 of device 1300 are provided above with reference to second electrode 1217, e.g., a metal, a metal alloy, a transition metal oxide, a transparent conductive oxide, a carbon material, a doped semiconductor, a binary semiconductor, a ternary semiconductor, or a conductive polymer such as described above with reference to FIG. 12A.

In some examples, the ion-selective membrane is in direct contact with the first electrode. In other examples, such as illustrated in FIG. 13, sensor 1310 further may include a solid contact layer 1313 disposed between the first electrode 1311 and the ion-selective membrane 1312. Solid contact layer 1313 may perform the function of enhancing the reproducibility and stability of the EMF by converting the signal into a measurable electrical potential signal. Additionally, or alternatively, solid contact layer 1313 may inhibit transport of water from the biological fluid 10 to the first electrode 1311 and/or accumulation of water at the first electrode 1311. Solid contact layer 1313 may include any suitable material or combination of materials. Nonlimiting example materials for use in solid contact layer 1313 are provided above with reference to second electrode 1217, e.g., a metal, a carbon material, a doped semiconductor, or a conductive polymer such as described above with reference to FIG. 12A. Alternatively, solid contact layer 1313 may include a redox couple which has a well-controlled concentration ratio of oxidized/reduced species that may be used to stabilize the interfacial electrical potential. The redox couple may include metallic centers with different oxidation states. Illustratively, the metallic centers may be selected from the group consisting of Co(II) and Co(III); Ir(II) and Ir(III); and Os(II) and Os(III). In alternative examples, the solid contact layer 213 may include a mixed conductor, or mixed ion-electron conductor, such as strontium titanate (SrTiO₃), titanium dioxide (TiO₂), (La,Ba,Sr)(Mn,Fe,Co)O_{3-d},La₂CuO_{4+d}, cerium(IV) oxide (CeO₂), lithium iron phosphate (LiFePO₄), and LiMnPO₄.

It will further be appreciated that sensor 1310 may have any suitable configuration. In the nonlimiting example illustrated in FIG. 13, substrate 1201 may be planar or substantially planar.

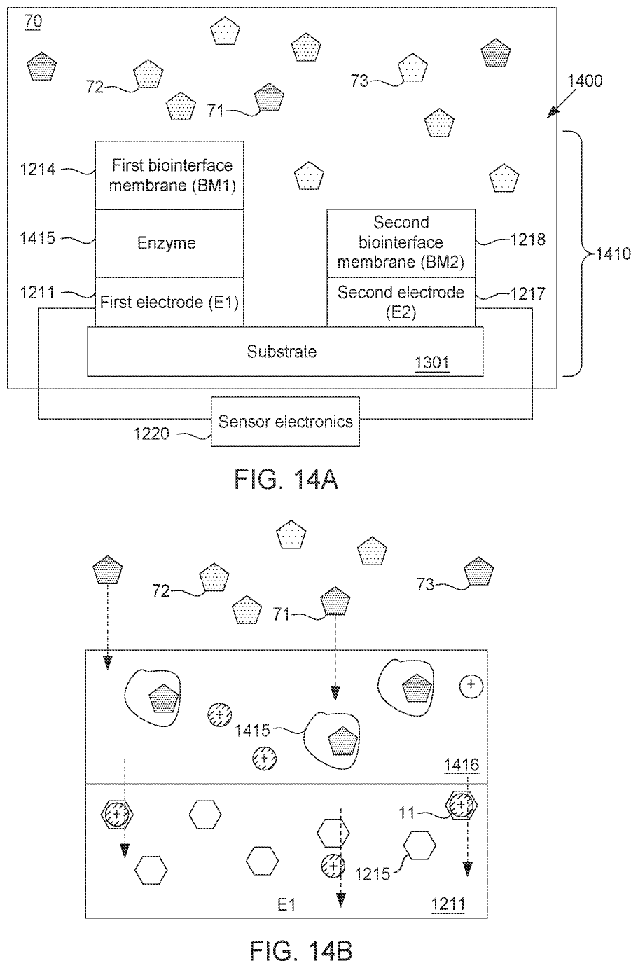

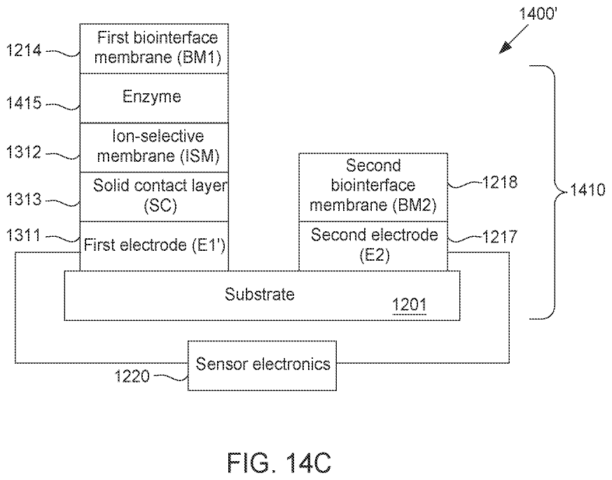

In the nonlimiting example illustrated in FIG. 14A, the ionophore may be located within first electrode (E1) 1211 disposed on the substrate and may be configured similarly as described with reference to FIG. 12A. Alternatively, in the nonlimiting example illustrated in FIG. 14C, the ionophore may be located within ion-selective membrane 1312 which may be configured in a manner such as described with reference to FIG. 13, and the first electrode 1311 may be configured in a manner such as described with reference to FIG. 13. First electrode 1211 or 1311 may be referred to as a working electrode (WE), while second electrode 1217 may be referred to as a reference electrode (RE).

The sensor electronics 1220 may be configured to generate a signal corresponding to an electromotive force (EMF). In some examples, the EMF is at least partially based on a potential difference that is generated between the first electrode and the second electrode responsive to the ionophore transporting the target ion to the first electrode. The sensor electronics 1220 may be configured to use the signal to generate an output corresponding to a measurement of the concentration of the target ion in the biological fluid, and/or may be configured to transmit the signal to an external device configured to use the signal to generate an output corresponding to a measurement of the concentration of the target ion in the biological fluid. Optionally, in some examples, the EMF is at least partially based on a potential difference that is generated between the first electrode and the second electrode responsive to biological fluid 10 conducting the electrophysiological signal to first electrode 111, and sensor electronics 1220 may be configured to use the signal to generate an output corresponding to a measurement of the electrophysiological signal.

In a manner such as illustrated in FIG. 14A, biological fluid 10 may include a plurality of analytes 71, 72, and 73. Device 1400 may be configured to measure the concentration of analyte 71, and accordingly such analyte may be referred to as a “target” analyte. As illustrated in FIG. 14B, enzyme 1415 may be located within enzyme layer 1416, and may selectively act upon target analyte 71 from biological fluid 10 or from biointerface membrane 1214 (if provided, e.g., as illustrated in FIG. 14A and configured similarly as described with reference to FIGS. 12A and 13). The action of enzyme 1415 upon the target analyte 71 generates the target ion 11. Ionophore 1215 within first electrode 1211 or within ion-selective membrane 1312 may selectively transport, or selectively bind, target ions 11 from enzyme 1415 to and within first electrode 1211 or first electrode 1311.

It will be appreciated that target analyte 71 may be any suitable analyte, enzyme 1415 may be any suitable enzyme that generates a suitable ion responsive to action upon that analyte, and ionophore 1215 may be any suitable ionophore that selectively transports and/or binds that ion generated by enzyme 1415 so as to generate an EMF based upon which the concentration of analyte 71 may be determined (whether using sensor electronics 1220 or an external device to which the sensor electronics 1220 transmits the electrophysiological signal and/or signal corresponding to ion concentration). Nonlimiting examples of analytes, enzymes, and ionophores that may be used together are listed below in Table 1.

TABLE 1

| Analyte | Enzyme | Ion generated | Ionophore |
|---|---|---|---|
| Urea | Urease | Ammonium | Nonactin |
| Glucose | Glucose | H+ (via peroxide) | Tridodecylamine, 4- |
| | oxidase | | Nonadecylpyridine, N,N- |
| | | | Dioctadecylmethylamine, |
| | | | Octadecyl isonicotinate, |
| | | | Calix[4]-aza-crown |
| Creatinine | Creatinine | Ammonium | Nonactin |
| | deaminase | | |
| Lactate | Lactate | H+ (via peroxide) | Tridodecylamine, 4- |
| | oxidase | | Nonadecylpyridine, N,N- |
| | | | Dioctadecylmethylamine, |
| | | | Octadecyl isonicotinate, |
| | | | Calix[4]-aza-crown |
| Cholesterol | Cholesterol | H+ (via peroxide) | Tridodecylamine, 4- |
| | oxidase | | Nonadecylpyridine, N,N- |
| | | | Dioctadecylmethylamine, |
| | | | Octadecyl isonicotinate, |
| | | | Calix[4]-aza-crown |
| Glutamate | Glutamate | Ammonium | Nonactin |
| | oxidase/ | | |
| | Glutamate | | |
| | dehydrogenase | | |
| Galactose | Galactose/ | H+ (via peroxide) | Tridodecylamine, 4- |
| | oxidase | | Nonadecylpyridine, N,N- |
| | | | Dioctadecylmethylamine, |
| | | | Octadecyl isonicotinate, |
| | | | Calix[4]-aza-crown |

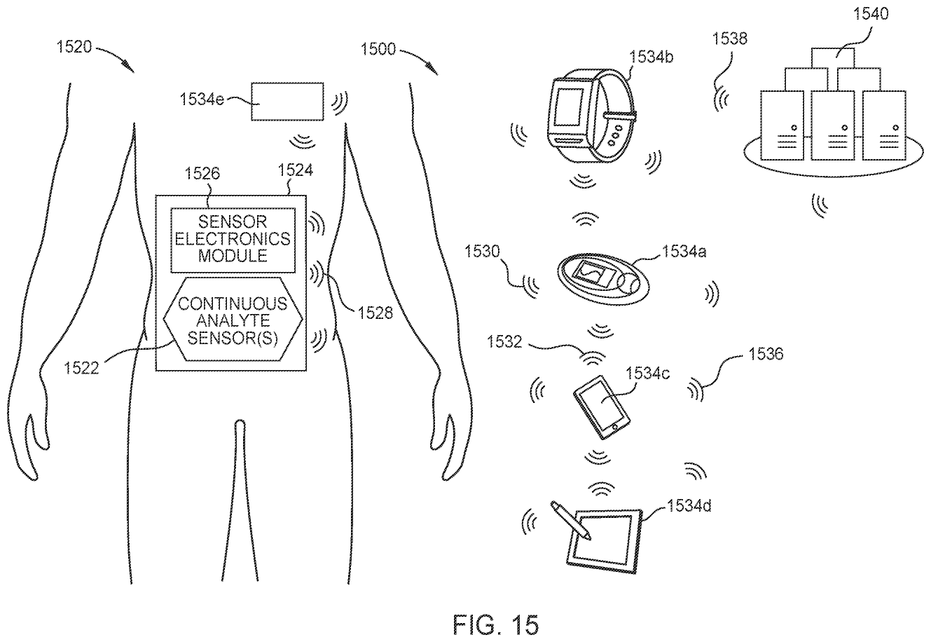

FIG. 15 is a diagram depicting an example continuous analyte monitoring system 1500 configured to measure one or more target ions and/or other analytes as discussed herein. The monitoring system 1500 includes an analyte sensor system 1524 operatively connected to a host 1520 and a plurality of display devices 1534 a-e according to certain aspects of the present disclosure. It should be noted that the display device 1534e alternatively or in addition to being a display device, may be a medicament delivery device that can act cooperatively with the analyte sensor system 1524 to deliver medicaments to host 1520. The analyte sensor system 1524 may include a sensor electronics module 1526 and a continuous analyte sensor 1522 associated with the sensor electronics module 1526. The sensor electronics module 1526 may be in direct wireless communication with one or more of the plurality of the display devices 1534a-e via wireless communications signals.

As will be discussed in greater detail below, display devices 1534a-e may also communicate amongst each other and/or through each other to analyte sensor system 1524. For ease of reference, wireless communications signals from analyte sensor system 1524 to display devices 1534a-e can be referred to as “uplink” signals 1528. Wireless communications signals from, e.g., display devices 1534a-e to analyte sensor system 1524 can be referred to as “downlink” signals 1530. Wireless communication signals between two or more of display devices 1534a-e may be referred to as “crosslink” signals 1532. Additionally, wireless communication signals can include data transmitted by one or more of display devices 1534a-d via “long-range” uplink signals 1536 (e.g., cellular signals) to one or more remote servers 1540 or network entities, such as cloud-based servers or databases, and receive long-range downlink signals 1538 transmitted by remote servers 1540.

The sensor electronics module 1526 includes sensor electronics that are configured to process sensor information and generate transformed sensor information. In certain embodiments, the sensor electronics module 1526 includes electronic circuitry associated with measuring and processing data from continuous analyte sensor 1522, including prospective algorithms associated with processing and calibration of the continuous analyte sensor data. The sensor electronics module 1526 can be integral with (non-releasably attached to) or releasably attachable to the continuous analyte sensor 1522 achieving a physical connection therebetween. The sensor electronics module 1526 may include hardware, firmware, and/or software that enables analyte level measurement. For example, the sensor electronics module 1526 can include a potentiostat, a power source for providing power to continuous analyte sensor 1522, other components useful for signal processing and data storage, and a telemetry module for transmitting data from itself to one or more display devices 1534a-e. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor. Examples of systems and methods for processing sensor analyte data are described in more detail herein and in U.S. Pat. Nos. 7,310,544 and 6,931,327 and U.S. Patent Publication Nos. 2005/0043598, 2007/0032706, 2007/0016381, 2008/0033254, 2005/0203360, 2005/0154271, 2005/0192557, 2006/0222566, 2007/0203966 and 2007/0208245, each of which are incorporated herein by reference in their entirety for all purposes.

Display devices 1534a-e are configured for displaying, alarming, and/or basing medicament delivery on the sensor information that has been transmitted by the sensor electronics module 1526 (e.g., in a customized data package that is transmitted to one or more of display devices 1534a-e based on their respective preferences). Each of the display devices 1534a-e can include a display such as a touchscreen display for displaying sensor information to a user (most often host 1520 or a caretaker/medical professional) and/or receiving inputs from the user. In some embodiments, the display devices 1534a-e may include other types of user interfaces such as a voice user interface instead of or in addition to a touchscreen display for communicating sensor information to the user of the display device 1534a-e and/or receiving user inputs. In some embodiments, one, some or all of the display devices 1534a-e are configured to display or otherwise communicate the sensor information as it is communicated from the sensor electronics module 1526 (e.g., in a data package that is transmitted to respective display devices 1534a-e), without any additional prospective processing required for calibration and real-time display of the sensor information.

In the embodiment of FIG. 15, one of the plurality of display devices 1534a-e may be a custom display device 1534a specially designed for displaying certain types of displayable sensor information associated with analyte values received from the sensor electronics module 1526 (e.g., a numerical value and an arrow, in some embodiments). In some embodiments, one of the plurality of display devices 1534a-e may be a handheld device 1534c, such as a mobile phone based on the Android, iOS operating system or other operating system, a palm-top computer and the like, where handheld device 1534c may have a relatively larger display and be configured to display a graphical representation of the continuous sensor data (e.g., including current and historic data). Other display devices can include other hand-held devices, such as a tablet 1534d, a smart watch 1534b, a medicament delivery device 1534e, a blood glucose meter, and/or a desktop or laptop computer.

As discussed above, because the different display devices 1534a-e provide different user interfaces, content of the data packages (e.g., amount, format, and/or type of data to be displayed, alarms, and the like) can be customized (e.g., programmed differently by the manufacture and/or by an end user) for each particular display device and/or display device type. Accordingly, in the embodiment of FIG. 12A, one or more of display devices 1534a-e can be in direct or indirect wireless communication with the sensor electronics module 1526 to enable a plurality of different types and/or levels of display and/or functionality associated with the sensor information, which is described in more detail elsewhere herein.

### Additional Considerations

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, acc, b-b, b-b-b, b-b-c, c-c, and c-c-c or any other ordering of a, b, and c).

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.”

While various examples of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various example examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described example examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “comprising as used herein is synonymous with “including,” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific examples and examples described herein, but rather to also cover all modification and alternatives coming with the true scope and spirit of the invention.

1. A monitoring system, comprising:
a continuous analyte sensor configured to generate analyte measurements associated with analyte levels of a patient; and
a sensor electronics module coupled to the continuous analyte sensor and configured to receive and process the analyte measurements.

2. The monitoring system of claim 1, wherein the continuous analyte sensor comprises:
a substrate,
a working electrode disposed on the substrate, and
a reference electrode disposed on the substrate, wherein the analyte measurements generated by the continuous analyte sensor correspond to an electromotive force at least in part based on a potential difference generated between the working electrode and the reference electrode.

3. The monitoring system of claim 1, wherein:
the continuous analyte sensor comprises a continuous glucose sensor, and
the analyte measurements include glucose measurements.

4. The monitoring system of claim 3, further comprising:
a memory comprising executable instructions; and
one or more processors in data communication with the memory and configured to execute the executable instructions to:
receive the glucose measurements from the sensor electronics module, wherein the glucose measurements comprise:
a first set of glucose measurements associated with one or more pre-treatment periods,
a second set of glucose measurements associated with one or more treatment periods, or
a third set of glucose measurements associated with one or more post-treatment periods;
process the glucose measurements to determine:
a first one or more glucose metrics associated with changes in the first set of glucose measurements,
a second one or more glucose metrics associated with changes in the second set of glucose measurements, or
a third one or more glucose metrics associated with changes in the third set of glucose measurements,
create one or more physiological profiles comprising:
a pre-treatment physiological profile corresponding to the first one or more glucose metrics,
a treatment physiological profile corresponding to the second one or more glucose metrics, or
a post-treatment physiological profile corresponding to the third one or more glucose metrics;
determine that the patient is in a period corresponding to a pre-treatment period, treatment period, or post-treatment period;
determine a likelihood of an adverse health event based on:
the determined period,
at least one of the pre-treatment physiological profile, the treatment physiological profile, or post-treatment physiological profile, or
a plurality of glucose measurements associated with at least one of the determined period or a period before the determined period; and
generate at least one of: one or more recommendations or optimized treatment parameters based on the likelihood.

5. The monitoring system of claim 4, further comprising:
one or more non-analyte sensors, wherein the processor is further configured to:
receive non-analyte sensor data generated for the patient using one or more non-analyte sensors, wherein:
the pre-treatment physiological profile, treatment physiological profile, and the post-treatment physiological profile are created further based on the non-analyte sensor data, and
the determined likelihood is further based on a set of non-analyte sensor data associated with the determined period or a period before the determined period.

6. The monitoring system of claim 5, wherein the one or more non-analyte sensors comprise at least one of an insulin pump, a haptic sensor, an ECG sensor, a heart rate monitor, a blood pressure sensor, a respiratory sensor, a peritoneal dialysis machine, or a hemodialysis machine.

7. The monitoring system of claim 4, wherein the glucose metric comprises a glucose rate of change.

8. The monitoring system of claim 4, wherein the physiological profiles correspond to patterns of corresponding glucose metrics.

9. The monitoring system of claim 4, wherein the adverse health event includes at least one of: hypokalemia, hyperkalemia, hypoglycemia, hyperglycemia, a cardiac event, or mortality.

10. The monitoring system of claim 4, wherein optimized treatment parameters comprise at least one of: a type of treatment, a dosage of treatment, an activity rate, an activity duration, an activity timing, or an optimized treatment parameter for dialysis.

11. The monitoring system of claim 10, wherein the optimized treatment parameter for dialysis comprises at least one of: a type of dialysate composition, a type of dialysate concentration, a type of dialysis membrane, a flow rate, a timing of treatment, a frequency of treatment, or a length of treatment.

12. The monitoring system of claim 4, wherein the processor is further configured to control operations of a medical device using one or more of the optimized treatment parameters.

13. The monitoring system of claim 4, wherein the one or more recommendations or optimized treatment parameters are generated using a model trained based on population data including records of historical patients indicating various treatment parameters corresponding to various treatments.

14. The monitoring system of claim 1, wherein:
the continuous analyte sensor comprises a continuous potassium sensor, and
the analyte measurements include potassium measurements.

15. The monitoring system of claim 14, further comprising:
a memory comprising executable instructions; and
one or more processors in data communication with the memory and configured to execute the executable instructions to:
receive the potassium measurements from the sensor electronics module, wherein the potassium measurements comprise:
a first set of potassium measurements associated with one or more pre-treatment periods,
a second set of potassium measurements associated with one or more treatment periods, or
a third set of potassium measurements associated with one or more post-treatment periods;
process the potassium measurements to determine:
a first one or more potassium metrics associated with changes in the first set of potassium measurements,
a second one or more potassium metrics associated with changes in the second set of potassium measurements, or
a third one or more potassium metrics associated with changes in the third set of potassium measurements;
create one or more physiological profiles comprising:
a pre-treatment physiological profile corresponding to the first one or more potassium metrics,
a treatment physiological profile corresponding to the second one or more potassium metrics, or
a post-treatment physiological profile corresponding to the third one or more potassium metrics;
determine that the patient is in a period corresponding to a pre-treatment period, treatment period, or post-treatment period;
determine a likelihood of an adverse health event based on:
the determined period,
at least one of the pre-treatment physiological profile, the treatment physiological profile, or post-treatment physiological profile, or
a plurality of potassium measurements associated with at least one of the determined period or a period before the determined period; and
generate at least one of: one or more recommendations or optimized treatment parameters based on the likelihood.

16. The monitoring system of claim 15, further comprising:
one or more non-analyte sensors, wherein the processor is further configured to:
receive non-analyte sensor data generated for the patient using one or more non-analyte sensors, wherein:
the pre-treatment physiological profile, treatment physiological profile, and the post-treatment physiological profile are created further based on the non-analyte sensor data, and
the determined likelihood is further based on a set of non-analyte sensor data associated with the determined period or a period before the determined period.

17. The monitoring system of claim 16, wherein the one or more non-analyte sensors comprise at least one of an insulin pump, a haptic sensor, an ECG sensor, a heart rate monitor, a blood pressure sensor, a respiratory sensor, a peritoneal dialysis machine, or a hemodialysis machine.

18. The monitoring system of claim 15, wherein the potassium metric comprises a potassium rate of change.

19. The monitoring system of claim 15, wherein optimized treatment parameters comprise at least one of: a type of treatment, a dosage of treatment, an activity rate, an activity duration, an activity timing, or an optimized treatment parameter for dialysis.

20. The monitoring system of claim 19, wherein the optimized treatment parameter for dialysis comprises at least one of: a type of dialysate composition, a type of dialysate concentration, a type of dialysis membrane, a flow rate, a timing of treatment, a frequency of treatment, or a length of treatment.
